(19) United States
(12) Patent Application Publication
(10) Pub. No.: US 2021/0171958 A1
(43) Pub. Date: Jun. 10, 2021

(54) METHODS OF TREATING CANCER

(71) Applicant: Foghorn Therapeutics Inc., Cambridge, MA (US)

(72) Inventors: Ho Man CHAN, Carlisle, MA (US); Qianhe ZHOU, Winchester, MA (US); Luis SOARES, Cambridge, MA (US)

(21) Appl. No.: 17/270,170

(22) PCT Filed: Aug. 23, 2019
(86) PCT No.: PCT/US2019/048002

§ 371 Date: Feb. 22, 2021

Related U.S. Application Data
(60) Provisional application No. 62/722,079, filed on Aug. 23, 2018.

(51) Int. Cl. C12N 15/113; A61P 35/00; C12N 9/22; A61K 45/06

(52) U.S. Cl. CPC C12N 15/1137; A61P 35/00; C12Y 306/04012; A61K 45/06; C12N 9/22

(57) ABSTRACT

The present disclosure features useful methods to treat cancer having a deficiency in ARID1 A and/or mismatch repair deficiency, e.g., in a subject in need thereof. In some embodiments, the methods described herein are useful in the treatment of cancer in combination with immunotherapies.

### BACKGROUND

Cancer remains one of the deadliest threats to human health and is the second leading cause of mortality. In 2012, there were an estimated 14.1 million cases of cancer diagnosed around the world and 8.2 million cancer deaths. By 2030, the global burden is expected to reach 21.6 million new cancer cases and 13.0 million cancer deaths annually. Thus, there is a need to develop new approaches for the treatment of cancer.

### SUMMARY OF THE INVENTION

The present invention features methods to treat cancer having ARID1A mutations and cancers with mutations in other subunits of the BAF complex. The present invention also features methods to treat cancer having a mismatch repair deficiency (MMRd), e.g., in a subject in need thereof. In some embodiments, the methods described herein are useful in the treatment of cancer in combination with immunotherapies.

In one aspect, the invention features a method of treating cancer having a mutation that results in a loss of function of AT-Rich Interaction Domain 1A (ARID1A) in a subject in need thereof. This method includes administering to the subject an effective amount of an agent that reduces the level and/or activity of Werner Syndrome RecQ Like Helicase (WRN) in a cell in the subject. In some embodiments, the activity of WRN is WRN helicase activity.

In another aspect, the invention features a method of reducing the level and/or activity of WRN in a cancer cell having a mutation that results in a loss of function of ARID1A in a subject in need thereof. This method includes administering to the subject an effective amount of an agent that reduces the level and/or activity of WRN in a cell in the subject. In some embodiments, the activity of WRN is WRN helicase activity.

In another aspect, the invention features a method of reducing tumor growth of a cancer having a mutation that results in a loss of function of ARID1A in a subject in need thereof. This method includes administering to the subject an effective amount of an agent that reduces the level and/or activity of WRN in a cell in the subject. In some embodiments, the activity of WRN is WRN helicase activity.

In another aspect, the invention features a method of treating cancer having a MMRd in a subject in need thereof. This method includes administering to the subject an effective amount of an agent that reduces the level and/or activity of WRN in a cell in the subject. In some embodiments, the activity of WRN is WRN helicase activity.

In another aspect, the invention features a method of reducing the level and/or activity of WRN in a cancer cell having a MMRd in a subject. This method includes contacting the cell with an effective amount of an agent that reduces the level and/or activity of WRN in the cell. In some embodiments, the activity of WRN is WRN helicase activity.

In another aspect, the invention features a method of reducing tumor growth of a cancer having a MMRd in a subject in need thereof. This method includes administering to the subject an effective amount of an agent that reduces the level and/or activity of WRN in a cell in the subject. In some embodiments, the activity of WRN is WRN helicase activity. In some embodiments, the MMRd is caused by a mutation in the MLH1, MLH3, MSH2, MSH3, MSH6, PMS1, PMS2, and/or EPCAM genes. In some embodiments, the MMRd is associated with a mutation in the MLH1, MSH2, MSH6, PMS2, and/or EPCAM genes. In some embodiments, the MMRd is associated with a mutation in the MLH1 gene. In some embodiments, the cancer has a microsatellite instability (MSI)-positive or MSI-high (MSI-H) phenotype. In some embodiments, the MSI-positive phenotype is characterized by the presence of an MSI at least one of the mononucleotide or dinucleotide markers selected from the group consisting of BAT25, BAT26, D2S123, D5S346, and D17S250. In some embodiments, the MSI-positive phenotype is an MSI-high (MSI-H) phenotype characterized by the presence of an MSI at least two of the mononucleotide or dinucleotide markers selected from the group consisting of BAT25, BAT26, D2S123, D5S346, and D17S250. Methods of identifying MSI-positive, MSI-H, or MMRd tumor status are well known in the art and include, e.g., polymerase chain reaction (PCR) tests for MSI-positive and MSI-H status or immunohistochemistry (IHC) tests for MMRd. In some embodiments, the cancer has an additional mutation that results in a loss of function of ARID1A.

In another aspect, the invention features a method of treating cancer having an MSI-positive phenotype in a subject in need thereof. This method includes administering to the subject an effective amount of an agent that reduces the level and/or activity of WRN in a cell in the subject. In some embodiments, the activity of WRN is WRN helicase activity.

In another aspect, the invention features a method of reducing the level and/or activity of WRN in a cancer cell having a microsatellite instability (MSI)-positive phenotype in a subject in need thereof. This method includes administering to the subject an effective amount of an agent that reduces the level and/or activity of WRN in a cell in the subject. In some embodiments, the activity of WRN is WRN helicase activity.

In another aspect, the invention features a method of reducing tumor growth of a cancer having a microsatellite instability (MSI)-positive phenotype in a subject in need thereof. This method includes administering to the subject an effective amount of an agent that reduces the level and/or activity of WRN in a cell in the subject. In some embodiments, the activity of WRN is WRN helicase activity. In some embodiments, the MSI-positive phenotype characterized by the presence of an MSI at least one of the mononucleotide or dinucleotide markers selected from the group consisting of BAT25, BAT26, D2S123, D5S346, and D17S250. In some embodiments, the MSI-positive phenotype is an MSI-high (MSI-H) phenotype characterized by the presence of an MSI at least two of the mononucleotide or dinucleotide markers selected from the group consisting of BAT25, BAT26, D2S123, D5S346, and D17S250. In some embodiments, MMRd is caused by a mutation in the MLH1, MLH3, MSH2, MSH3, MSH6, PMS1, PMS2, and/or EPCAM genes. In some embodiments, the MMRd is associated with a mutation in the MLH1, MSH2, MSH6, PMS2, and/or EPCAM genes. In some embodiments, the MMRd is associated with a mutation in the MLH1 gene. Methods of identifying MSI-positive, MSI-H, or MMRd tumor status are well known in the art and include, e.g., polymerase chain reaction (PCR) tests for MSI-positive and MSI-H status or immunohistochemistry (IHC) tests for MMRd. In some embodiments, the cancer has a MMRd. In some embodiments, the cancer has an additional mutation that results in a loss of function of ARID1A.

In some embodiments of any of the above aspects, the agent that reduces the level and/or activity of WRN in a cell in the subject is directed to or targets one or more domains of WRN selected from the group consisting of a helicase domain, an endonuclease domain, a RecQ C-terminal (RQC) domain, and/or a C-terminal helix-turn-helix (HTH) motif. In some embodiments, the agent that reduces the level and/or activity of WRN in a cell in the subject is directed to or targets a WRN helicase domain. In some embodiments, the agent that reduces the level and/or activity of WRN in a cell in the subject is directed to or targets a WRN endonuclease domain. In some embodiments, the agent that reduces the level and/or activity of WRN in a cell in the subject is directed to or targets a WRN RQC domain. In some embodiments, the agent that reduces the level and/or activity of WRN in a cell in the subject is directed to or targets a WRN HTH motif. In some embodiments, the agent that reduces the level and/or activity of WRN in a cell in the subject inhibits the nuclear localization of WRN. In some embodiments, the agent that reduces the level and/or activity of WRN in a cell in the subject inhibits WRN mRNA translation. In some embodiments, the agent that reduces the level and/or activity of WRN in a cell in the subject destabilizes WRN mRNA. In some embodiments, the agent that reduces the level and/or activity of WRN in a cell in the subject inhibits WRN mRNA transcription.

In some embodiments of any of the above aspects, the agent that reduces the level and/or activity of WRN is a nuclease. In some embodiments, the agent that reduces the level and/or activity of WRN is a polynucleotide. In some embodiments, the agent that reduces the level and/or activity of WRN is a small molecule compound. In some embodiments, the agent that reduces the level and/or activity of WRN is an antibody. In some embodiments, the agent that reduces the level and/or activity of WRN is an enzyme. In some embodiments, the nuclease is a clustered regularly interspaced short palindromic repeats (CRISPR)-associated protein. In some embodiments, the nuclease is a transcription activator-like effector nuclease (TALEN). In some embodiments, the nuclease is a meganuclease. In some embodiments, the nuclease is a zinc finger nuclease (ZFN). In some embodiments, the polynucleotide is an antisense nucleic acid. In some embodiments, the polynucleotide is a CRISPR/Cas 9 nucleotide. In some embodiments, the polynucleotide is a short interfering RNA (siRNA). In some embodiments, the polynucleotide is a short hairpin RNA (shRNA). In some embodiments, the polynucleotide is a micro RNA (miRNA). In some embodiments, the polynucleotide is a ribozyme. In some embodiments, the polynucleotide comprises a sequence having at least 70% sequence identity (e.g., 70%, 75%, 80%, 85%, 86%, 87%, 88%, 89%, 90%, 91%, 92%, 93%, 94%, 95%, 96%, 97%, 98%, 99%, 99.9% identity, or more) to the nucleic acid sequence of any one of SEQ ID NOs: 5-50. In some embodiments, the polynucleotide comprises a sequence having at least 70% sequence identity (e.g., 70%, 75%, 80%, 85%, 86%, 87%, 88%, 89%, 90%, 91%, 92%, 93%, 94%, 95%, 96%, 97%, 98%, 99%, 99.9% identity, or more) to the nucleic acid sequence of any one of SEQ ID NOs: 5-10.

In some embodiments of any of the above aspects, the method further includes administering to the subject an anti-cancer therapy. In some embodiments, the agent that reduces the level and/or activity of WRN is administered prior to the anti-cancer therapy. In some embodiments, the agent that reduces the level and/or activity of WRN is administered simultaneously with the anti-cancer therapy. In some embodiments, the agent that reduces the level and/or activity of WRN is administered after the anti-cancer therapy. In some embodiments, the anti-cancer therapy is an immunotherapy. In some embodiments, the immunotherapy is a CTLA-4 inhibitor. In some embodiments, the immunotherapy is a PD-1 inhibitor. In some embodiments, the immunotherapy is a PD-L1 inhibitor. In some embodiments, the immunotherapy is adoptive T cell transfer therapy (e.g., CAR-T therapy). In some embodiments, the anti-cancer therapy is a non-drug treatment (e.g., radiological therapy or a surgical procedure). In some embodiments, the anti-cancer therapy is a chemotherapy.

In some embodiments of any of the above aspects, the agent that reduces the level and/or activity of WRN is administered systemically or intratumorally to the subject.

In some embodiments of any of the above aspects, the cancer is an MSI-positive cancer, an MSI-H cancer, an adrenocortical carcinoma, a bladder carcinoma, a breast carcinoma, a cervical squamous cell carcinoma, an endocervical adenocarcinoma, a cholangiocarcinoma, a chronic lymphocytic leukemia, a colorectal cancer (e.g., a colon adenocarcinoma), a cutaneous T-cell lymphoma, a lymphoid neoplasm diffuse large B-cell lymphoma, an esophageal carcinoma, a glioblastoma multiforme, a head and neck squamous cell carcinoma, a kidney chromophobe, a kidney renal papillary cell carcinoma, an acute myeloid leukemia, a lower-grade glioma, a liver hepatocellular carcinoma, a lung adenocarcinoma, a lung squamous cell carcinoma, a mesothelioma, a nasopharyngeal carcinoma, an ovarian cancer (e.g., an ovarian serous cystadenocarcinoma), a pancreatic adenocarcinoma, a pheochromocytoma, paraganglioma, a prostate adenocarcinoma, a rectal adenocarcinoma, a sarcoma, a skin cutaneous melanoma, a stomach adenocarcinoma, a testicular germ cell tumor, a thyroid carcinoma, a thymoma, an uterine corpus endometrial carcinoma, an uterine carcinosarcoma, an uveal melanoma, a pediatric acute myeloid leukemia, a pediatric neuroblastoma, or a pediatric high-risk Wilms tumor. In some embodiments, the cancer is an MSI-positive cancer, an MSI-H cancer, an ovarian cancer, a uterine corpus endometrial carcinoma, a colorectal cancer (e.g., a colon adenocarcinoma), or a stomach adenocarcinoma.

In some embodiments of any of the above aspects, the subject is a human.

In another aspect, the invention features a kit including a pharmaceutical composition including an agent that reduces the level and/or activity of WRN in a cell in a subject and a package insert with instructions to perform any of the methods described herein. In some embodiments, the kit additionally includes an additional therapeutic agent (e.g., an anti-cancer agent).

### Definitions

In this application, unless otherwise clear from context, (i) the term “a” may be understood to mean “at least one”; (ii) the term “or” may be understood to mean “and/or”; and (iii) the terms “including” and “includes” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a therapeutic agent as described herein, e.g., an anti-WRN agent) to a subject or system. Administration to an animal subject (e.g., to a human) may be by any appropriate route. For example, in some embodiments, administration may be systemic (including intravenous), intratumoral, bronchial, buccal, enteral, interdermal, intra-arterial, intradermal, intragastric, intramedullary, intramuscular, intranasal, intraperitoneal, intrathecal, intraventricular, mucosal, nasal, oral, rectal, subcutaneous, sublingual, topical, tracheal, transdermal, vaginal, or vitreal.

The term “cancer” refers to a condition caused by the proliferation of malignant neoplastic cells, such as tumors, neoplasms, carcinomas, sarcomas, leukemias, and lymphomas.

As used herein, “mismatch repair deficiency,” “MMRd,” and “mismatch repair deficient” refer to a defect in the mismatch repair (MMR) system resulting in impaired MMR function. The MMR system is a group of proteins that are involved in recognizing and repairing base pair mismatches and single strand insertion/deletion loops arising in the genome by various mechanisms during the replication, recombination, or chemical modification of DNA. The loss of MMR activity can occur through a number of mechanisms including loss of the chromosomes that the genes are on that encode the proteins, mutations in the genes, and degradation of the enzyme(s) involved. The defect can be associated with a mutation, e.g., in one or more of the MSH2, MLH1, MSH6, PMS2, and/or EPCAM genes. A MMRd that is “associated with,” for example, a mutation, refers to a MMRd that is mediated, at least in part, by a mutation in, e.g., the MLH1, MSH2, MSH6, PMS2, and/or EPCAM genes. AT-rich interaction domain-containing protein 1A (ARID1A) may also be associated with MMRd.

“Microsatellite instability” or “MSI” as used herein, is defined as alterations in the lengths of microsatellites due to deletion or insertion of repeating units to produce novel length alleles in tumor DNA when compared with the normal/germline DNA from the same individual. A tumor that has an “MSI-positive” phenotype is a tumor that has an MSI at least one (e.g., an MSI-positive cancer, or a low-frequency MSI cancer) of the evaluated mononucleotide or dinucleotide loci (e.g., BAT25, BAT26, D2S123, D5S346, and D17S250). High frequency MSI (MSI-H), or an MSI-H phenotype is characterized by an instability in at least two of the evaluated markers. Methods of identifying MSI-positive or MSI-H tumor status are well known in the art and include, e.g., polymerase chain reaction (PCR) tests for MSI status. Mononucleotide or dinucleotide markers used for the characterization of MSI status include, but are not limited to, BAT25, BAT26, D2S123, D5S346, and D17S250; also known as the Bethesda panel.

As used herein, a “combination therapy” and “administered in combination” mean that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

By “level” is meant a level or activity of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, or ng/mL) or percentage relative to total protein or mRNA in a sample.

As used herein, the term “WRN” refers to Werner syndrome ATP-dependent helicase, a member of the RecQ subfamily of DNA helicase proteins involved in DNA replication, DNA damage repair, and telomere maintenance. WRN is encoded by the WRN gene. The amino acid sequence of an exemplary protein encoded by human WRN is shown under UniProt Accession No. Q14191-1 or in SEQ ID NO: 1. The nucleic acid sequence of an exemplary human WRN is shown under NCBI Reference Sequence: NM_000553.5 or in SEQ ID NO: 2. The term “WRN” also refers to natural variants of the wild-type WRN protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type WRN, which is set forth in SEQ ID NO: 1.

By “reducing the activity of WRN” is meant decreasing the level of an activity related to a WRN, or a related downstream effect. The activity level of WRN may be measured using any method known in the art. In some embodiments, an agent which reduces the activity of WRN is a polynucleotide. In some embodiments, an agent which reduces the activity of WRN is a nuclease.

By “reducing the level of WRN” is meant decreasing the level of WRN in a cell or subject, e.g., by administering a polynucleotide to the cell or subject. The level of WRN may be measured using any method known in the art.

As used herein, the term “ARID1A” refers to AT-rich interaction domain-containing protein 1A, a member of the SWI/SNF family, whose members have helicase and ATPase activities and are thought to regulate transcription of certain genes by altering the surrounding chromatin structure. ARID1A is encoded by the ARID1A gene. The amino acid sequence of an exemplary protein encoded by human ARID1A is shown under UniProt Accession No. 014497-1 or in SEQ ID NO: 3. The nucleic acid sequence of an exemplary human ARID1A is shown under NCBI Reference Sequence: NM_006015.5 or in SEQ ID NO: 4. The term “ARID1A” also refers to natural variants of the wild-type ARID1A protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type ARID1A, which is set forth in SEQ ID NO: 3.

As used herein, the terms “WRN inhibitor” and “anti-WRN agent” refer to any agent which reduces the level and/or activity of WRN. Non-limiting examples of anti-WRN agents include nucleases, polynucleotides (e.g., siRNA), small molecule compounds, antibodies, and enzymes.

As used herein, the terms “effective amount,” “therapeutically effective amount,” and “a “sufficient amount” of an agent that reduces the level and/or activity of WRN in a cell in a subject described herein refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of WRN in a cell in a subject sufficient to achieve a treatment response as compared to the response obtained without administration of the agent that reduces the level and/or activity of WRN (e.g., WRN helicase activity). The amount of a given agent that reduces the level and/or activity of WRN described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent that reduces the level and/or activity of WRN of the present disclosure is an amount which results in a beneficial or desired result in a subject as compared to a control. As defined herein, a therapeutically effective amount of an agent that reduces the level and/or activity of WRN of the present disclosure may be readily determined by one of ordinary skill by routine methods known in the art. Dosage regimen may be adjusted to provide the optimum therapeutic response.

As used herein, the term “reducing tumor growth” refers to an inhibition or a reduction in tumor growth or metastasis of a cancer as compared to its growth prior to treatment. The reduction of tumor growth may be a reduction of about 5% or greater (e.g., 5%, 10%, 20%, 30%, 40%, 50%, 60%, 70%, 80%, 90%, or greater), and can be measured by any suitable means known in the art.

The term “inhibitory RNA agent” refers to an RNA, or analog thereof, having sufficient sequence complementarity to a target RNA to direct RNA interference. Examples also include a DNA that can be used to make the RNA. RNA interference (RNAi) refers to a sequence-specific or selective process by which a target molecule (e.g., a target gene, protein, or RNA) is down-regulated. Generally, an interfering RNA (“iRNA”) is a double-stranded short-interfering RNA (siRNA), short hairpin RNA (shRNA), or single-stranded micro-RNA (miRNA) that results in catalytic degradation of specific mRNAs, and also can be used to lower or inhibit gene expression.

The terms “short interfering RNA” and “siRNA” (also known as “small interfering RNAs”) refer to an RNA agent, preferably a double-stranded agent, of about 10-50 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., Dicer or a homolog thereof).

The term “shRNA,” as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The terms “miRNA” and “microRNA” refer to an RNA agent, preferably a single-stranded agent, of about 10-50 nucleotides in length, preferably between about 15-25 nucleotides in length, which is capable of directing or mediating RNA interference. Naturally-occurring miRNAs are generated from stem-loop precursor RNAs (i.e., pre-miRNAs) by Dicer. The term “Dicer,” as used herein, includes Dicer as well as any Dicer ortholog or homolog capable of processing dsRNA structures into siRNAs, miRNAs, siRNA-like or miRNA-like molecules. The term microRNA (“miRNA”) is used interchangeably with the term “small temporal RNA” (“stRNA”) based on the fact that naturally-occurring miRNAs have been found to be expressed in a temporal fashion (e.g., during development).

The term “antisense,” as used herein, refers to a nucleic acid comprising a polynucleotide that is sufficiently complementary to all or a portion of a gene, primary transcript, or processed mRNA, so as to interfere with expression of the endogenous gene (e.g., WRN). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

The term “antisense nucleic acid” includes single-stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity (e.g., 80%, 85%, 86%, 87%, 88%, 89%, 90%, 91%, 92%, 93%, 94%, 95%, 96%, 97%, 98%, 99%, 99.9% identity, or more) with the targeted polypeptide sequence (e.g., a WRN polypeptide sequence). The antisense nucleic acid can be complementary to an entire coding strand, or to only a portion thereof. In some embodiments, an antisense nucleic acid molecule is antisense to a “coding region” of the coding strand of a nucleotide sequence. The term “coding region” refers to the region of the nucleotide sequence comprising codons that are translated into amino acid residues. In some embodiments, the antisense nucleic acid molecule is antisense to a “noncoding region” of the coding strand of a nucleotide sequence. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of mRNA, or can be antisense to only a portion of the coding or noncoding region of an mRNA. For example, the antisense oligonucleotide can be complementary to the region surrounding the translation start site. An antisense oligonucleotide can be, for example, about 5, 10, 15, 20, 25, 30, 35, 40, 45, or 50 nucleotides in length.

“Percent (%) sequence identity,” with respect to a reference polynucleotide or polypeptide sequence, is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software, such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “sample” refers to a specimen (e.g., a tissue sample (e.g., a tumor tissue sample), cells, urine, blood, saliva, amniotic fluid, or cerebrospinal fluid) isolated from a subject.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value, such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a therapeutic agent described herein; a sample from a subject that has been treated by a therapeutic agent described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); or a subject that has been treated with a therapeutic agent described herein. In preferred embodiments, the reference sample, standard, or level is matched to the sample subject sample by at least one of the following criteria: age, weight, sex, disease stage, and overall health. A standard curve of levels of a purified protein, e.g., as described herein, within the normal reference range can also be used as a reference.

As used interchangeably herein, the terms “subject,” “patient,” and “individual” refer to any organism to which a therapeutic agent in accordance with the invention may be administered, e.g., for experimental, diagnostic, prophylactic, and/or therapeutic purposes. Typical subjects include any animal (e.g., mammals, such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilization of the (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

The term “PD-1 inhibitor,” as used herein, refers to a compound, such as an antibody capable of inhibiting the activity of the protein that in humans is encoded by the PDCD1 gene (Accession No. Q15116). Known PD-1 inhibitors include nivolumab, pembrolizumab, pidilizumab, and BMS 936559.

The term “PD-L1 inhibitor,” as used herein, refers to a compound, such as an antibody capable of inhibiting the activity of the protein that in humans is encoded by the CD274 gene (Accession No. Q9NZQ7). Known PD-L1 inhibitors include atezolizumab (TECENTRIQ®), avelumab (BAVENCIO®), and durvalumab (IMFINZI®; MED14736) and Cemiplimab.

The term “CTLA-4 inhibitor,” as used herein, refers to a compound, such as an antibody capable of inhibiting the activity of the protein that in humans is encoded by the CTLA4 gene (Accession No. P16410). One known CTLA-4 inhibitor is ipilimumab.

The details of one or more embodiments of the invention are set forth in the description below. Other features, objects, and advantages of the invention will be apparent from the description and from the claims.

### BRIEF DESCRIPTION OF THE DRAWINGS

FIGS. 1A-1B is a series of images illustrating the effects of doxycycline (Dox)-inducible sgRNA-directed depletion of WRN on cell growth in the ARID1A-mutant RKO (FIG. 1A) and the ARID1A-wild type H1299 (FIG. 1B) cells. PCNA serves as a positive control for cell growth inhibition. Six independent sgRNAs against WRN were tested in each cell line.

FIG. 1C is an immunoblot illustrating the effects of Dox-inducible sgRNA-directed depletion of WRN in RKO and H1299 cells on WRN protein levels. BAF155 serves as a positive loading control.

FIG. 2A is a graph showing the Euclidean distance profile from the cell line mutations for the HEC6 cell line to the Catalog of Somatic Mutations in Cancer (COSMIC) context signatures.

FIG. 2B is a graph showing the average Euclidean distance profile for all WRN-dependent cell lines compiled from the Cancer Cell Line Encyclopedia (CCLE) to COSMIC context signatures.

FIG. 3A is a flow chart illustrating the steps of the analysis utilized for the identification of tumors dependent on WRN protein. A random-forest classifier was created based on WRN dependency using K-means clustering and the distance to COSMIC mutation signatures. The resulting classifier was used to classify tumor samples regarding WRN dependency using distances from the COSMIC mutation signatures for each sample.

FIG. 3B is a graph illustrating the average difference to COSMIC mutation signatures of predicted WRN-dependent cases. Average differences to signature contexts were calculated for the predicted cases and sorted; dark color indicates MMRd-associated signatures.

FIG. 3C is a graph illustrating the distribution of predicted WRN-dependent tumors according to tissue of origin.

FIG. 3D is a graph illustrating ARID1A mutation status of predicted WRN-dependent cases. Tumors were classified according to the impact of the ARID1A mutation, if present.

FIG. 3E is a graph illustrating the differential gene expression of uterine tumors. Uterine tumors were classified according to predicted WRN dependency. Differential gene expression was calculated using RNA-seq data from uterine tumors.

FIG. 4A is a series of images illustrating the effects of Dox-inducible sgRNA-directed depletion of WRN on cell growth in the MMR-deficient HCT116 cells. PCNA serves as a positive control for cell growth inhibition. Six independent sgRNAs against WRN were tested.

FIG. 4B is an immunoblot illustrating the effects of Dox-inducible sgRNA-directed depletion of WRN in HCT116 cells on WRN protein levels. BAF155 serves as a positive loading control.

FIG. 5A is a series of images illustrating the effects of various WRN rescue constructs on RKO cells with Dox-inducible sgWRN-1-mediated WRN depletion.

FIG. 5B is a diagram of the WRN WT, WRN E84A, and WRN K577M rescue constructs.

FIG. 5C is an immunoblot illustrating the expression levels of WRN WT, WRN E84A, and WRN K577M constructs. Beta-actin was used as a loading control.

FIG. 6A is a series of immunoblots illustrating the effect of WRN depletion by TMP treatment on ecDHFR-WRN HCT116 cells. Tubulin was used as a loading control.

FIG. 6B is a series of immunoblots illustrating the effect of WRN depletion by asunaprevir (ASV) on RKO-SMASh-WRN cells. Tubulin was used as a loading control.

FIG. 7A is a graph illustrating the effect of WRN depletion by inducible CRISPR on tumor growth in HCT116 xenograft models.

FIG. 7B is an immunoblot illustrating the effect of partial WRN protein depletion by inducible CRISPR in a HCT116 xenograft model. Actin was used as a loading control.

FIG. 7C is a pair of graphs illustrating the effect of WRN depletion by SMASh degron on tumor growth in RKO xenograft models.

FIG. 7D is an immunoblot illustrating the effect of WRN protein depletion by SMASh degron in a RKO xenograft model. Actin was used as a loading control.

FIG. 8A is a mean-difference (MD) plot and a volcano plot of differential expressed genes upon WRN CRISPR induction. Genes differentially expressed with adjusted P-value below 0.05 are highlighted in black, and non-significantly changed genes are highlighted in grey.

FIG. 8B is a table illustrating the top three significantly enriched sets obtained from gene set enrichment analysis (GSEA) of gene expression changes upon WRN CRISPR induction in RKO cell line (False Discovery Rate<0.05).

FIG. 9A is an MD plot and a volcano plot of differentially expressed genes following WRN CRISPR induction the HCT116 cell line. Genes differentially expressed with adjusted P-value below 0.05 are highlighted in black, and non-significantly changed genes are highlighted in grey.

FIG. 9B is a table illustrating the top three significantly enriched sets obtained from GSEA of gene expression changes upon WRN CRISPR induction in the HCT116 cell line (False Discovery Rate<0.05).

FIG. 10A is an MD plot and a volcano plot of differentially expressed genes in xenografted tumors upon ASV treatment of animals implanted with a RKO parental cell line or a RKO SMASh-WRN engineered cell line. Genes differentially expressed with adjusted P-value below 0.05 are highlighted in black, non-significantly changed genes in grey. The number of differentially expressed genes indicated in bottom table.

FIG. 10B is a table illustrating the top five significantly enriched sets obtained from gene set enrichment analysis of gene expression changes upon ASV induced WRN degradation in xenografted tumors.

FIG. 11 is a series of graphs illustrating the effect of specific sgRNAs targeting of WRN in various cell lines. WRN CRISPR guides are indicated by circles, empty circles correspond guides that map to defined WRN functional domains. Functional domains along the WRN protein are indicated in the bottom of each plot. The grey shaded area corresponds to the log 2 dropout ratio of non-targeting guide RNAs between 0.25 and the 99.75 percentile.

### DETAILED DESCRIPTION

The present inventors have found that reducing the level and/or activity of the Werner Syndrome RecQ Like Helicase (WRN) in cancer cells having a mutation that results in a loss of function of AT-Rich Interaction Domain 1A (ARID1A) and/or a mismatch repair deficiency (MMRd) inhibits the proliferation of the cancer cells. Accordingly, the invention features methods for reducing the level and/or activity of WRN for the treatment of cancer, e.g., in a subject in need thereof. Exemplary methods are described herein.

### Mismatch Repair Deficiency and Cancer

Human tumors develop through two major pathways of genome instability: chromosomal instability and microsatellite instability (MSI) that results from defects in the DNA mismatch repair (MMR) system.

The MMR system is a DNA integrity maintenance system. The main role of MMR proteins is the correction of single base nucleotide mismatches (insertions or deletions) generated during DNA replication and recombination, thus maintaining the genomic stability. The mechanism of MMR involves at least three different processes: recognition, excision, and resynthesis. Recognition of single base replication errors is performed by the MutSα (MSH2-MSH6 heteroduplex) or MutSβ (MSH2-MSH3 heteroduplex), excision of the lagging strand from the mismatch by one of the MutL complexes (mainly MutLα formed by MLH1/PMS2) recruited by MutS protein, and resynthesis of the excised-DNA and ligation by DNA polymerase delta and DNA ligase I.

Loss of expression of one of the MMR proteins may result from inherited germline defects in one of the mismatch repair genes; rarely both of the inherited alleles are mutated as in constitutional MMR deficiency syndrome leading to cancer in early childhood (called constitutional mismatch repair deficiency). More frequently, only one mutated allele is inherited and loss of the other allele occurs somatically, as in Lynch syndrome, an autosomal dominant condition that predisposes an individual to cancer development (particularly colorectal cancer, ovarian cancer, and endometrial cancer). Alternatively, MMR deficiency may be derived by either somatic mutation or methylation of one of the MMR genes: sporadic MMR deficient tumors are often the result of epigenetic silencing of MLH1 promoter due to a hypermethylation mechanism. MMRd in cancer is characterized by mutations in one or more mismatch repair genes including MSH2, MLH1, MSH6, PMS2, and EPCAM.

Due to its role in genomic stability, MMRd leads to accumulation of somatic mutations. Microsatellites—repetitive short (1-6 base pairs) tandem DNA sequences scattered throughout the whole genome—are particularly subject to copying errors when mismatch repair is compromised. Therefore, MMRd can be determined by examining the microsatellites; when they are demonstrated to be hypermutated (instable), MMRd may be deducted. MSI is encountered in 15% of colorectal cancers and a variety of extracolonic malignancies showing a deficient DNA mismatch repair system, including endometrial cancers, gastric cancers, small bowel cancers, and tumors of other organs. MMRd in cancer can be characterized, e.g., by the presence of an MSI at least one (e.g., an MSI-positive cancer, or a low-frequency MSI cancer) of the mononucleotide or dinucleotide markers BAT25, BAT26, D2S123, D5S346, and D17S250; also known as the Bethesda panel. High frequency MSI (MSI-H) is characterized by an instability in at least two of the five markers.

MMR status of a tumor may be assessed either by immunohistochemistry (IHC) that tests loss of a MMR protein, or by PCR-based assays for microsatellite instability. Methods of determining MMR status of a tumor are well known in the art.

### WRN

WRN is a member of the RecQ subfamily of DNA helicase proteins, involved in DNA replication, DNA damage repair (including repair of double strand breaks by homologous recombination or non-homologous end joining, repair of single nucleotide damages by base excision repair), and telomere maintenance. It is also required for normal replication fork progression after DNA damage or fork arrest. WRN is the only RecQ Helicase that contains 3′ to 5′ exonuclease activity. These exonuclease activities include degradation of recessed 3′ ends and initiation of DNA degradation from a gap in double-stranded DNA.

Defects in this gene are the cause of Werner syndrome, an autosomal recessive disorder characterized by accelerated aging and an elevated risk for certain cancers including soft tissue sarcomas, osteosarcoma, thyroid cancer, and melanoma. Wild-type human WRN (UNIPROT reference number: Q14191-1) has the amino acid sequence of:

(SEQ ID NO: 1)
MSEKKLETTAQQRKCPEWMNVQNKRCAVEERKACVRKSVFEDDLPFLEFT
GSIVYSYDASDCSFLSEDISMSLSDGDVVGFDMEWPPLYNRGKLGKVALI
QLCVSESKCYLFHVSSMSVFPQGLKMLLENKAVKKAGVGIEGDQWKLLRD
FDIKLKNFVELTDVANKKLKCTETWSLNSLVKHLLGKQLLKDKSIRCSNW
SKFPLTEDQKLYAATDAYAGFIIYRNLEILDDTVQRFAINKEEEILLSDM
NKQLTSISEEVMDLAKHLPHAFSKLENPRRVSILLKDISENLYSLRRMII
GSTNIETELRPSNNLNLLSFEDSTTGGVQQKQIREHEVLIHVEDETWDPT
LDHLAKHDGEDVLGNKVERKEDGFEDGVEDNKLKENMERACLMSLDITEH
ELQILEQQSQEEYLSDIAYKSTEHLSPNDNENDTSYVIESDEDLEMEMLK
HLSPNDNENDTSYVIESDEDLEMEMLKSLENLNSGTVEPTHSKCLKMERN
LGLPTKEEEEDDENEANEGEEDDDKDFLWPAPNEEQVTCLKMYFGHSSFK
PVQWKVIHSVLEERRDNVAVMATGYGKSLCFQYPPVYVGKIGLVISPLIS
LMEDQVLQLKMSNIPACFLGSAQSENVLTDIKLGKYRIVYVTPEYCSGNM
GLLQQLEADIGITLIAVDEAHCISEWGHDFRDSFRKLGSLKTALPMVPIV
ALTATASSSIREDIVRCLNLRNPQITCTGFDRPNLYLEVRRKTGNILQDL
QPFLVKTSSHWEFEGPTIIYCPSRKMTQQVTGELRKLNLSCGTYHAGMSF
STRKDIHHRFVRDEIQCVIATIAFGMGINKADIRQVIHYGAPKDMESYYQ
EIGRAGRDGLQSSCHVLWAPADINLNRHLLTEIRNEKFRLYKLKMMAKME
KYLHSSRCRRQIILSHFEDKQVQKASLGIMGTEKCCDNCRSRLDHCYSMD
DSEDTSWDFGPQAFKLLSAVDILGEKFGIGLPILFLRGSNSQRLADQYRR
HSLFGTGKDQTESWWKAFSRQLITEGFLVEVSRYNKFMKICALTKKGRNW
LHKANTESQSLILQANEELCPKKLLLPSSKTVSSGTKEHCYNQVPVELST
EKKSNLEKLYSYKPCDKISSGSNISKKSIMVQSPEKAYSSSQPVISAQEQ
ETQIVLYGKLVEARQKHANKMDVPPAILATNKILVDMAKMRPTTVENVKR
IDGVSEGKAAMLAPLLEVIKHFCQTNSVQTDLFSSTKPQEEQKTSLVAKN
KICTLSQSMAITYSLFQEKKMPLKSIAESRILPLMTIGMHLSQAVKAGCP
LDLERAGLTPEVQKIIADVIRNPPVNSDMSKISLIRMLVPENIDTYLIHM
AIEILKHGPDSGLQPSCDVNKRRCFPGSEEICSSSKRSKEEVGINTETSS
AERKRRLPVWFAKGSDTSKKLMDKTKRGGLFS

Wild-type human WRN (GenBank accession number: NM_000553.5) has the nucleic acid sequence of:

(SEQ ID NO: 2)
CAGCCGCCCCTCCTGCGGCCGCTGCGGGGGCCGCCGCCTGACTTCGGACACCGGCCCCG
CACCCGCCAGGAGGGGAGGGAAGGGGAGGCGGGGAGAGCGACGGCGGGGGGCGGGCG
GTGGACCCCGCCTCCCCCGGCACAGCCTGCTGAGGGGAAGAGGGGGTCTCCGCTCTTCCT
CAGTGCACTCTCTGACTGAAGCCCGGCGCGTGGGGTGCAGCGGGAGTGCGAGGGGACTG
GACAGGTGGGAAGATGGGAATGAGGACCGGGCGGCGGGAATGTTCTCACTTCTCCGGATT
CCACCGGGATGCAGGACTCTAGCTGCCCAGCCGCACCTGCGAAGAGACTACACTTCCCGA
GGTGCTCAGCGGCAGCGAGGGCCTCCACGCATGCGCACCGCGGCGCGCTGGGCGGGGCT
GGATGGGCTGTGGTGGGAGGGTTGCAGCGCCGCGAGAAAGGCGAGCCGGGCCGGGGGC
GGGGAAAGGGGTGGGGCAGGAACGGGGGCGGGGACGGCGCTGGAGGGGCGGGTCGGGT
AGGTCTCCCGGAGCTGATGTGTACTGTGTGCGCCGGGGAGGCGCCGGCTTGTACTCGGCA
GCGCGGGAATAAAGTTTGCTGATTTGGTGTCTAGCCTGGATGCCTGGGTTGCAGGCCCTGC
TTGTGGTGGCGCTCCACAGTCATCCGGCTGAAGAAGACCTGTTGGACTGGATCTTCTCGGG
TTTTCTTTCAGATATTGTTTTGTATTTACCCATGAAGACATTGTTTTTTGGACTCTGCAAATAG
GACATTTCAAAGATGAGTGAAAAAAAATTGGAAACAACTGCACAGCAGCGGAAATGTCCTGA
ATGGATGAATGTGCAGAATAAAAGATGTGCTGTAGAAGAAAGAAAGGCATGTGTTCGGAAGA
GTGTTTTTGAAGATGACCTCCCCTTCTTAGAATTCACTGGATCCATTGTGTATAGTTACGATG
CTAGTGATTGCTCTTTCCTGTCAGAAGATATTAGCATGAGTCTATCAGATGGGGATGTGGTG
GGATTTGACATGGAGTGGCCACCATTATACAATAGAGGGAAACTTGGCAAAGTTGCACTAAT
TCAGTTGTGTGTTTCTGAGAGCAAATGTTACTTGTTCCACGTTTCTTCCATGTCAGTTTTTCC
CCAGGGATTAAAAATGTTGCTTGAAAATAAAGCAGTTAAAAAGGCAGGTGTAGGAATTGAAG
GAGATCAGTGGAAACTTCTACGTGACTTTGATATCAAATTGAAGAATTTTGTGGAGTTGACAG
ATGTTGCCAATAAAAAGCTGAAATGCACAGAGACCTGGAGCCTTAACAGTCTGGTTAAACAC
CTCTTAGGTAAACAGCTCCTGAAAGACAAGTCTATCCGCTGTAGCAATTGGAGTAAATTTCCT
CTCACTGAGGACCAGAAACTGTATGCAGCCACTGATGCTTATGCTGGTTTTATTATTTACCGA
AATTTAGAGATTTTGGATGATACTGTGCAAAGGTTTGCTATAAATAAAGAGGAAGAAATCCTA
CTTAGCGACATGAACAAACAGTTGACTTCAATCTCTGAGGAAGTGATGGATCTGGCTAAGCA
TCTTCCTCATGCTTTCAGTAAATTGGAAAACCCACGGAGGGTTTCTATCTTACTAAAGGATAT
TTCAGAAAATCTATATTCACTGAGGAGGATGATAATTGGGTCTACTAACATTGAGACTGAACT
GAGGCCCAGCAATAATTTAAACTTATTATCCTTTGAAGATTCAACTACTGGGGGAGTACAACA
GAAACAAATTAGAGAACATGAAGTTTTAATTCACGTTGAAGATGAAACATGGGACCCAACACT
TGATCATTTAGCTAAACATGATGGAGAAGATGTACTTGGAAATAAAGTGGAACGAAAAGAAG
ATGGATTTGAAGATGGAGTAGAAGACAACAAATTGAAAGAGAATATGGAAAGAGCTTGTTTG
ATGTCGTTAGATATTACAGAACATGAACTCCAAATTTTGGAACAGCAGTCTCAGGAAGAATAT
CTTAGTGATATTGCTTATAAATCTACTGAGCATTTATCTCCCAATGATAATGAAAACGATACGT
CCTATGTAATTGAGAGTGATGAAGATTTAGAAATGGAGATGCTTAAGCATTTATCTCCCAATG
ATAATGAAAACGATACGTCCTATGTAATTGAGAGTGATGAAGATTTAGAAATGGAGATGCTTA
AGTCTTTAGAAAACCTCAATAGTGGCACGGTAGAACCAACTCATTCTAAATGCTTAAAAATGG
AAAGAAATCTGGGTCTTCCTACTAAAGAAGAAGAAGAAGATGATGAAAATGAAGCTAATGAA
GGGGAAGAAGATGATGATAAGGACTTTTTGTGGCCAGCACCCAATGAAGAGCAAGTTACTTG
CCTCAAGATGTACTTTGGCCATTCCAGTTTTAAACCAGTTCAGTGGAAAGTGATTCATTCAGT
ATTAGAAGAAAGAAGAGATAATGTTGCTGTCATGGCAACTGGATATGGAAAGAGTTTGTGCT
TCCAGTATCCACCTGTTTATGTAGGCAAGATTGGCCTTGTTATCTCTCCCCTTATTTCTCTGA
TGGAAGACCAAGTGCTACAGCTTAAAATGTCCAACATCCCAGCTTGCTTCCTTGGATCAGCA
CAGTCAGAAAATGTTCTAACAGATATTAAATTAGGTAAATACCGGATTGTATACGTAACTCCA
GAATACTGTTCAGGTAACATGGGCCTGCTCCAGCAACTTGAGGCTGATATTGGTATCACGCT
CATTGCTGTGGATGAGGCTCACTGTATTTCTGAGTGGGGGCATGATTTTAGGGATTCATTCA
GGAAGTTGGGCTCCCTAAAGACAGCACTGCCAATGGTTCCAATCGTTGCACTTACTGCTACT
GCAAGTTCTTCAATCCGGGAAGACATTGTACGTTGCTTAAATCTGAGAAATCCTCAGATCAC
CTGTACTGGTTTTGATCGACCAAACCTGTATTTAGAAGTTAGGCGAAAAACAGGGAATATCCT
TCAGGATCTGCAGCCATTTCTTGTCAAAACAAGTTCCCACTGGGAATTTGAAGGTCCAACAA
TCATCTACTGTCCTTCTAGAAAAATGACACAACAAGTTACAGGTGAACTTAGGAAACTGAATC
TATCCTGTGGAACATACCATGCGGGCATGAGTTTTAGCACAAGGAAAGACATTCATCATAGG
TTTGTAAGAGATGAAATTCAGTGTGTCATAGCTACCATAGCTTTTGGAATGGGCATTAATAAA
GCTGACATTCGCCAAGTCATTCATTACGGTGCTCCTAAGGACATGGAATCATATTATCAGGA
GATTGGTAGAGCTGGTCGTGATGGACTTCAAAGTTCTTGTCACGTCCTCTGGGCTCCTGCAG
ACATTAACTTAAATAGGCACCTTCTTACTGAGATACGTAATGAGAAGTTTCGATTATACAAATT
AAAGATGATGGCAAAGATGGAAAAATATCTTCATTCTAGCAGATGTAGGAGACAAATCATCTT
GTCTCATTTTGAGGACAAACAAGTACAAAAAGCCTCCTTGGGAATTATGGGAACTGAAAAAT
GCTGTGATAATTGCAGGTCCAGATTGGATCATTGCTATTCCATGGATGACTCAGAGGATACA
TCCTGGGACTTTGGTCCACAAGCATTTAAGCTTTTGTCTGCTGTGGACATCTTAGGCGAAAA
ATTTGGAATTGGGCTTCCAATTTTATTTCTCCGAGGATCTAATTCTCAGCGTCTTGCCGATCA
ATATCGCAGGCACAGTTTATTTGGCACTGGCAAGGATCAAACAGAGAGTTGGTGGAAGGCTT
TTTCCCGTCAGCTGATCACTGAGGGATTCTTGGTAGAAGTTTCTCGGTATAACAAATTTATGA
AGATTTGCGCCCTTACGAAAAAGGGTAGAAATTGGCTTCATAAAGCTAATACAGAATCTCAG
AGCCTCATCCTTCAAGCTAATGAAGAATTGTGTCCAAAGAAGTTGCTTCTGCCTAGTTCGAAA
ACTGTATCTTCGGGCACCAAAGAGCATTGTTATAATCAAGTACCAGTTGAATTAAGTACAGAG
AAGAAGTCTAACTTGGAGAAGTTATATTCTTATAAACCATGTGATAAGATTTCTTCTGGGAGT
AACATTTCTAAAAAAAGTATCATGGTACAGTCACCAGAAAAAGCTTACAGTTCCTCACAGCCT
GTTATTTCGGCACAAGAGCAGGAGACTCAGATTGTGTTATATGGCAAATTGGTAGAAGCTAG
GCAGAAACATGCCAATAAAATGGATGTTCCCCCAGCTATTCTGGCAACAAACAAGATACTGG
TGGATATGGCCAAAATGAGACCAACTACGGTTGAAAACGTAAAAAGGATTGATGGTGTTTCT
GAAGGCAAAGCTGCCATGTTGGCCCCTCTGTTGGAAGTCATCAAACATTTCTGCCAAACAAA
TAGTGTTCAGACAGACCTCTTTTCAAGTACAAAACCTCAAGAAGAACAGAAGACGAGTCTGG
TAGCAAAAAATAAAATATGCACACTTTCACAGTCTATGGCCATCACATACTCTTTATTCCAAGA
AAAGAAGATGCCTTTGAAGAGCATAGCTGAGAGCAGGATTCTGCCTCTCATGACAATTGGCA
TGCACTTATCCCAAGCGGTGAAAGCTGGCTGCCCCCTTGATTTGGAGCGAGCAGGCCTGAC
TCCAGAGGTTCAGAAGATTATTGCTGATGTTATCCGAAACCCTCCCGTCAACTCAGATATGA
GTAAAATTAGCCTAATCAGAATGTTAGTTCCTGAAAACATTGACACGTACCTTATCCACATGG
CAATTGAGATCCTTAAACATGGTCCTGACAGCGGACTTCAACCTTCATGTGATGTCAACAAAA
GGAGATGTTTTCCCGGTTCTGAAGAGATCTGTTCAAGTTCTAAGAGAAGCAAGGAAGAAGTA
GGCATCAATACTGAGACTTCATCTGCAGAGAGAAAGAGACGATTACCTGTGTGGTTTGCCAA
AGGAAGTGATACCAGCAAGAAATTAATGGACAAAACGAAAAGGGGAGGTCTTTTTAGTTAAG
CTGGCAATTACCAGAACAATTATGTTTCTTGCTGTATTATAAGAGGATAGCTATATTTTATTTC
TGAAGAGTAAGGAGTAGTATTTTGGCTTAAAAATCATTCTAATTACAAAGTTCACTGTTTATTG
AAGAACTGGCATCTTAAATCAGCCTTCCGCAATTCATGTAGTTTCTGGGTCTTCTGGGAGCC
TACGTGAGTACATCACCTAACAGAATATTAAATTAGACTTCCTGTAAGATTGCTTTAAGAAACT
GTTACTGTCCTGTTTTCTAATCTCTTTATTAAAACAGTGTATTTGGAAAATGTTATGTGCTCTG
ATTTGATATAGATAACAGATTAGTAGTTACATGGTAATTATGTGATATAAAATATTCATATATTA
TCAAAATTCTGTTTTGTAAATGTAAGAAAGCATAGTTATTTTACAAATTGTTTTTACTGTCTTTT
GAAGAAGTTCTTAAATACGTTGTTAAATGGTATTAGTTGACCAGGGCAGTGAAAATGAAACC
GCATTTTGGGTGCCATTAAATAGGGAAAAAACATGTAAAAAATGTAAAATGGAGACCAATTGC
ACTAGGCAAGTGTATATTTTGTATTTTATATACAATTTCTATTATTTTTCAAGTAATAAAACAAT
GTTTTTCATACTGAATATTATATATATATTTTTTAGCTTTCATTTACTTAATTATTTTAAGTACCT
TTATTTTTCCAGGATGTCAGAATTTGATTCTAATCTCTCTTATGTAGCACATGTGACTTAATTT
AAAACCTATACTGTGACACAGAGTTGGGTAAACGATGATTATTTAACTTTAAGCAGTTCACCA
TCCATTTCAAAGCCTTTGATTGGCTTTTTTGTAAATAAAAATAACTTGTTAAGAAACAAATATAT
CTGTCATAGAAGAACTAGAAAATCCAGGGAAGTGAGAAAAATGAAAATAAAAATCATTCATAG
TTTTACTAGTAGCTAATCACAGTCAACCTCTTTTGTGTATCCCACCAGACTTTTTTATATTCAT
TTGTTTTTAGTTAAAATATAAAAGTCTCGTATATTCCCATTTTTCTGCATTGCATTACCAGAAG
GTAGTGGCGCCTATTAAATATGTGATATGTTGTTGTCCAGCCATGGCTTCTGCATTTGCATGC
TTTTGTGTGTGCATCTGCAATACCCTGTGAATATCCTGTGTGATGGAGTGGCAAGTACGCAC
AGACACGTCTGCTGCATGCCTAGGTACGAGGCTGTCTCCAGGAGAAGCACTTGTTTGATTAT
TTGAGTTGCCAATTGAATTTGCTGCTTTTTTTCATGGCTTGCCATTTTCACTGAAAAGAATGAC
TAATGAAAAACGATGATTGGTTATTAGATTTGGATGTTTGGCAGACATTTTCTCAAAATTGAAC
TAAGTTGGCCTCTTCACGGAAAACAACTGGTATTTGTTGTGCCAATGATAAAATTGGAGATTT
CTAGCAAAATGTATAATTTTGGAAAAGTTGTGTTCCTCCACTGGAAGCTTGACAGCTTTCCTT
AACATAAAGACTTCTCTTTCTCTTCGCTTTCACTACTACTACTACTAATTCTTCTTCTGATTCTT
CTTCTTCTCCTTCTTCCTTCTTCCTTCCTTCCTCCTCCTCCTCCTTCTTCTTCCTCTTCCTCTT
CTTCTTTCTCTCTTTCCTTCCTTCCCTTCCCTTCCCCTTCCTTCCTTCCTTCCTTCCTTCCTCC
CTCCCTCCCTCCCTCCCTCCCTCCCTCCTTTCTTTTTCTTTCTCTTTCTTTCTTTCTTTCTCTCT
CTCTCTCTCTTTCTTTCTTTTTCTTTCTCTTTTTCTTTCTTTCAAGCAGTCCTCCCGCCTCAGTC
CCCCAAAATAGTGGGATTACAGGTGTGAGCCACCATGCACAGCCTTACATAAAGCCTTTTCT
AATGAGATGGATAGTAATTAACAAATGTGAGTTTTTGATATTATATAAAGATTTTTTCTGTGTTT
CGAAGATCCGTATAACTCAGTGAATCAGTATGTTCTGGATGACTAATATGTGATGTTAAGAAA
TCATGACTGAGGCCGGGCGCGGTGGCTCACGCCTGTAATCCCAGCACTTTGGGAGGCCGA
GGCGGGCGGATCACGAGATCAGGAGATCGAGACCACCCTGGCCAACATGGTGAAACCCCG
TCTCTACTAAAAATACAAAAATTAGCTGGGTGTGTTGGTGCGTGCCTATAATCCCAGCTACTC
GGGAGGCTGAGGCAGGAGAATCGCTTGAACTCAGGAGGCGGAGATTGCAGTGAGCTGAGA
CTGCGCCACTGCACCCCAGCCTGGCGACAGAGCAAGACTCCGTCTCAAAAATAAAAAAAGA
AATCATGACTGGGTAAAAGATCTGTTCAGAGTACAAGATGGACCAATGGATTTGATATATTTG
AATATAACAGAGTATGAAAAAGTTTATTGATATAGTTTCAGATTACACACTGCAACTAATCTTT
AAGAAACTATTACTTGTCCACTTTTTGGTAAAATTTCAGAGAACAATGTCCACCATTATCTGAA
CAGGCTATTAAAATACTCTTCTCTTTTCCAACTACGTGCCTGTGCAAAGTCAGATTTTTTTCAT
ATACTTCAGCCAAAACAGCATATCAAAATGGATTGAATGCAGAAGTAGATCTGAGAATACAG
CCACTTTTGTTAAGCCAGACAATGAGATTTGCAAAATGTAAACAATGCTGCTGTTCTCAGTTT
TTAAAAATATGTTTTTTAAAAGTATTTATGTTAATGTGTACTT.

### ARID1A

ARID1A is a member of the SWI/SNF family, whose members have helicase and ATPase activities, and are thought to regulate transcription of certain genes by altering the surrounding chromatin structure. The large ATP-dependent chromatin remodeling complex, SWI/SNF, is required for transcriptional activation of genes normally repressed by chromatin.

ARID1A is the most mutated chromatin remodeling protein in human cancers, with over a 50% mutation rate in ovarian clear cell carcinomas. There are no targeted therapies against ARID1A-mutated cancers. A large subset of ARID1A-mutated cancers, including endometrial, colorectal, and gastric cancer, is also highly correlated with MMRd. Wild-type human ARID1A (UNIPROT reference number: 014497-1) has the amino acid sequence of:

(SEQ ID NO: 3)
MAAQVAPAAASSLGNPPPPPPSELKKAEQQQREEAGGEAAAAAAAERGEM
KAAAGQESEGPAVGPMPLGKELQDGAESNGGGGGGGAGSGGGPGAEPDLK
NSNGNAGPRPALNNNLTEPPGGGGGGSSDGVGAPPHSAAAALPPPAYGFG
QPYGRSPSAVAAAAAAVFHQQHGGQQSPGLAALQSGGGGGLEPYAGPQQN
SHDHGFPNHQYNSYYPNRSAYPPPAPAYALSSPRGGTPGSGAAAAAGSKP
PPSSSASASSSSSSFAQQRFGAMGGGGPSAAGGGTPQPTATPTLNQLLTS
PSSARGYQGYPGGDYSGGPQDGGAGKGPADMASQCWGAAAAAAAAAAASG
GAQQRSHHAPMSPGSSGGGGQPLARTPQPSSPMDQMGKMRPQPYGGTNPY
SQQQGPPSGPQQGHGYPGQPYGSQTPQRYPMTMQGRAQSAMGGLSYTQQI
PPYGQQGPSGYGQQGQTPYYNQQSPHPQQQQPPYSQQPPSQTPHAQPSYQ
QQPQSQPPQLQSSQPPYSQQPSQPPHQQSPAPYPSQQSTTQQHPQSQPPY
SQPQAQSPYQQQQPQQPAPSTLSQQAAYPQPQSQQSQQTAYSQQRFPPPQ
ELSQDSFGSQASSAPSMTSSKGGQEDMNLSLQSRPSSLPDLSGSIDDLPM
GTEGALSPGVSTSGISSSQGEQSNPAQSPFSPHTSPHLPGIRGPSPSPVG
SPASVAQSRSGPLSPAAVPGNQMPPRPPSGQSDSIMHPSMNQSSIAQDRG
YMQRNPQMPQYSSPQPGSALSPRQPSGGQIHTGMGSYQQNSMGSYGPQGG
QYGPQGGYPRQPNYNALPNANYPSAGMAGGINPMGAGGQMHGQPGIPPYG
TLPPGRMSHASMGNRPYGPNMANMPPQVGSGMCPPPGGMNRKTQETAVAM
HVAANSIQNRPPGYPNMNQGGMMGTGPPYGQGINSMAGMINPQGPPYSMG
GTMANNSAGMAASPEMMGLGDVKLTPATKMNNKADGTPKTESKSKKSSSS
TTTNEKITKLYELGGEPERKMWVDRYLAFTEEKAMGMTNLPAVGRKPLDL
YRLYVSVKEIGGLTQVNKNKKWRELATNLNVGTSSSAASSLKKQYIQCLY
AFECKIERGEDPPPDIFAAADSKKSQPKIQPPSPAGSGSMQGPQTPOSTS
SSMAEGGDLKPPTPASTPHSQIPPLPGMSRSNSVGIQDAFNDGSDSTFQK
RNSMTPNPGYQPSMNTSDMMGRMSYEPNKDPYGSMRKAPGSDPFMSSGQG
PNGGMGDPYSRAAGPGLGNVAMGPRQHYPYGGPYDRVRTEPGIGPEGNMS
TGAPQPNLMPSNPDSGMYSPSRYPPQQQQQQQQRHDSYGNQFSTQGTPSG
SPFPSQQTTMYQQQQQNYKRPMDGTYGPPAKRHEGEMYSVPYSTGQGQPQ
QQQLPPAQPQPASQQQAAQPSPQQDVYNQYGNAYPATATAATERRPAGGP
QNQFPFQFGRDRVSAPPGTNAQQNMPPQMMGGPIQASAEVAQQGTMWQGR
NDMTYNYANRQSTGSAPQGPAYHGVNRTDEMLHTDQRANHEGSWPSHGTR
QPPYGPSAPVPPMTRPPPSNYQPPPSMQNHIPQVSSPAPLPRPMENRTSP
SKSPFLHSGMKMQKAGPPVPASHIAPAPVQPPMIRRDITFPPGSVEATQP
VLKQRRRLTMKDIGTPEAWRVMMSLKSGLLAESTWALDTINILLYDDNSI
MTFNLSQLPGLLELLVEYFRRCLIEIFGILKEYEVGDPGQRTLLDPGRFS
KVSSPAPMEGGEEEEELLGPKLEEEEEEEVVENDEEIAFSGKDKPASENS
EEKLISKFDKLPVKIVQKNDPFVVDCSDKLGRVQEFDSGLLHWRIGGGDT
TEHIQTHFESKTELLPSRPHAPCPPAPRKHVTTAEGTPGTTDQEGPPPDG
PPEKRITATMDDMLSTRSSTLTEDGAKSSEAIKESSKFPFGISPAQSHRN
IKILEDEPHSKDETPLCTLLDWQDSLAKRCVCVSNTIRSLSFVPGNDFEM
SKHPGLLLILGKLILLHHKHPERKQAPLTYEKEEEQDQGVSCNKVEWWWD
CLEMLRENTLVTLANISGQLDLSPYPESICLPVLDGLLHWAVCPSAEAQD
PFSTLGPNAVLSPQRLVLETLSKLSIQDNNVDLILATPPFSRLEKLYSTM
VRFLSDRKNPVCREMAVVLLANLAQGDSLAARAIAVQKGSIGNLLGFLED
SLAATQFQQSQASLLHMQNPPFEPTSVDMMRRAARALLALAKVDENHSEF
TLYESRLLDISVSPLMNSLVSQVICDVLFLIGQS

### Anti-WRN Agents

Agents described herein that reduce the level and/or activity of WRN in a cell in a subject may be, for example, a polynucleotide, a small molecule compound, an antibody, and/or an enzyme. The agents reduce the level of WRN, or reduce the level of an activity related to WRN (e.g., WRN helicase activity), and/or related downstream effect in a cell or subject. In some embodiments, the agents reduce or inhibit WRN helicase activity. In other embodiments, the agents reduce or inhibit WRN endonuclease activity.

In some embodiments, the agent that reduces the level and/or activity of WRN in a cell in a subject is directed to or targets a specific domain of WRN. In some embodiments, the agent is directed to or targets a WRN helicase domain. In other embodiments, the agent is directed to or targets a WRN endonuclease domain. In other embodiments, the agent is directed to or targets a WRN RecQ C-terminal (RQC) domain. In other embodiments, the agent is directed to or targets a WRN C-terminal helix-turn-helix (HTH) motif. In some embodiments, the agent that reduces the level and/or activity of WRN in a cell in the subject inhibits the nuclear localization of WRN. In some embodiments, the agent that reduces the level and/or activity of WRN in a cell in the subject inhibits WRN mRNA translation. In some embodiments, the agent that reduces the level and/or activity of WRN in a cell in the subject destabilizes WRN mRNA. In some embodiments, the agent that reduces the level and/or activity of WRN in a cell in the subject inhibits WRN mRNA transcription.

In some embodiments, the agent that reduces the level and/or activity of WRN in a cell in a subject is a polynucleotide, a small molecule compound, an antibody, and/or an enzyme (e.g., a nuclease).

Polynucleotides

In some embodiments, the agent that reduces the level and/or activity of WRN is a polynucleotide. In some embodiments, the agent that reduces the level and/or activity of WRN is an inhibitory RNA molecule, e.g., that acts by way of the RNA interference (RNAi) pathway. An inhibitory RNA molecule can decrease the expression level (e.g., protein level or mRNA level) of WRN. For example, an inhibitory RNA molecule includes a short interfering RNA (siRNA), a short hairpin RNA (shRNA), and/or a microRNA (miRNA) that targets full-length WRN. A siRNA is a double-stranded RNA molecule that typically has a length of about 19-25 base pairs. A shRNA is a RNA molecule including a hairpin turn that decreases expression of target genes via RNAi. A miRNA is a non-coding RNA molecule that typically has a length of about 22 nucleotides. miRNAs bind to target sites on mRNA molecules and silence the mRNA, e.g., by causing cleavage of the mRNA, destabilization of the mRNA, and/or inhibition of translation of the mRNA. Degradation is catalyzed by an enzymatic, RNA-induced silencing complex (RISC).

In some embodiments, the agent that reduces the level and/or activity of WRN in a cell in a subject is an antisense nucleic acid. Antisense nucleic acids include antisense RNA (asRNA) and antisense DNA (asDNA) molecules, typically about 10 to 30 nucleotides in length, which recognize polynucleotide target sequences or sequence portions through hydrogen bonding interactions with the nucleotide bases of the target sequences (e.g., WRN). The target sequences may be single- or double-stranded RNA, or single- or double-stranded DNA.

A polynucleotide of the invention can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity. The polynucleotides mentioned above may also be provided in a specialized form, such as liposomes or microspheres, or may be applied to gene therapy, or may be provided in combination with attached moieties. Such attached moieties include polycations, such as polylysine that act as charge neutralizers of the phosphate backbone, or hydrophobic moieties, such as lipids (e.g., phospholipids, cholesterols, etc.) that enhance the interaction with cell membranes or increase uptake of the nucleic acid. These moieties may be attached to the nucleic acid at the 3′ or 5′ ends and may also be attached through a base, sugar, or intramolecular nucleoside linkage. Other moieties may be capping groups specifically placed at the 3′ or 5′ ends of the nucleic acid to prevent degradation by nucleases, such as exonuclease, RNase, or other nucleases known in the art. Such capping groups include hydroxyl protecting groups known in the art, including glycols, such as polyethylene glycol and tetraethylene glycol. The inhibitory action of the polynucleotide can be examined using a cell-line or animal based gene expression system of the present invention in vivo and in vitro.

In some embodiments, the polynucleotide decreases the level and/or activity or function of WRN (e.g., WRN helicase activity). In embodiments, the polynucleotide inhibits expression of WRN. In other embodiments, the polynucleotide increases degradation of WRN and/or decreases the stability (i.e., half-life) of WRN. The polynucleotide can be chemically synthesized or transcribed in vitro.

Inhibitory polynucleotides can be designed by methods well known in the art. siRNA, miRNA, shRNA, and asRNA molecules with homology sufficient to provide sequence specificity required to uniquely degrade any RNA can be designed using programs known in the art, including, but not limited to, those maintained on websites for Thermo Fisher Scientific, the German Cancer Research Center, and The Ohio State University Wexner Medical Center. Systematic testing of several designed species for optimization of the inhibitory polynucleotide sequence can be routinely performed by those skilled in the art. Considerations when designing interfering polynucleotides include, but are not limited to, biophysical, thermodynamic, and structural considerations, base preferences at specific positions in the sense strand, and homology. The making and use of inhibitory therapeutic agents based on non-coding RNA, such as ribozymes, RNAse P, siRNAs, and miRNAs are also known in the art, for example, as described in Sioud, RNA Therapeutics: Function, Design, and Delivery (Methods in Molecular Biology). Humana Press 2010. Exemplary inhibitory polynucleotides, for use in the methods of the invention, are provided in Table 1, below. In some embodiments, the inhibitory polynucleotides have a nucleic acid sequence with at least 50% (e.g., at least 50%, at least 60%, at least 70%, at least 80%, at least 85%, at least 90%, at least 95%, at least 96%, at least 97%, at least 98%, at least 99%, or 100%) sequence identity to the nucleic acid sequence of an inhibitory polynucleotide in Table 1. In some embodiments, the inhibitory polynucleotides have a nucleic acid sequence with at least 85% sequence identity to the nucleic acid sequence of an inhibitory polynucleotide in Table 1. In some embodiments, the inhibitory polynucleotides have a nucleic acid sequence with at least 90% sequence identity to the nucleic acid sequence of an inhibitory polynucleotide in Table 1. In some embodiments, the inhibitory polynucleotides have a nucleic acid sequence with at least 95% sequence identity to the nucleic acid sequence of an inhibitory polynucleotide in Table 1.

Construction of vectors for expression of polynucleotides for use in the invention may be accomplished using conventional techniques which do not require detailed explanation to one of ordinary skill in the art. For generation of efficient expression vectors, it is necessary to have regulatory sequences that control the expression of the polynucleotide. These regulatory sequences include promoter and enhancer sequences and are influenced by specific cellular factors that interact with these sequences, and are well known in the art.

Gene Editing

In some embodiments, the agent that reduces the level and/or activity of WRN in a cell in a subject is a component of a gene-editing system. For example, the agent that reduces the level and/or activity of WRN introduces an alteration (e.g., insertion, deletion (e.g., knockout), translocation, inversion, single point mutation, or other mutation) in WRN. In some embodiments, the agent that reduces the level and/or activity of WRN in a cell in a subject is a nuclease. Exemplary gene editing systems include zinc finger nucleases (ZFNs), Transcription Activator-Like Effector-based Nucleases (TALENs), meganucleases, and the clustered regulatory interspaced short palindromic repeat (CRISPR) system. ZFNs, TALENs, and CRISPR-based methods are described, e.g., in Gaj et al., Trends Biotechnol. 31(7):397-405 (2013).

CRISPR refers to a set of (or system including a set of) clustered regularly interspaced short palindromic repeats. A CRISPR system refers to a system derived from CRISPR and Cas (a CRISPR-associated protein) or other nuclease that can be used to silence or mutate a gene described herein. The CRISPR system is a naturally-occurring system found in bacterial and archeal genomes. The CRISPR locus is made up of alternating repeat and spacer sequences. In naturally-occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482(7385):331-338 (2012). For example, such modification of the system includes introducing into a eukaryotic cell a plasmid containing a specifically-designed CRISPR and one or more appropriate Cas proteins. The CRISPR locus is transcribed into RNA and processed by Cas proteins into small RNAs that include a repeat sequence flanked by a spacer. The RNAs serve as guides to direct Cas proteins to silence specific DNA/RNA sequences, depending on the spacer sequence. See, e.g., Horvath et al., Science 327(5962):167-170 (2010); Makarova et al., Biology Direct 1:7 (2006); Pennisi, Science 341(6148):833-836 (2013). In some examples, the CRISPR system includes the Cas9 protein, a nuclease that cuts on both strands of the DNA. See, e.g., Id.

In some embodiments, in a CRISPR system for use described herein, e.g., in accordance with one or more methods described herein, the spacers of the CRISPR are derived from a target gene sequence, e.g., from a WRN sequence.

In some embodiments, the agent that reduces the level and/or activity of WRN includes a guide RNA (gRNA) for use in a CRISPR system for gene editing. Exemplary gRNAs, for use in the methods of the invention, are provided in Table 1, below. In some embodiments, the agent that reduces the level and/or activity of WRN includes a ZFN, or an mRNA encoding a ZFN, that targets (e.g., cleaves) a nucleic acid sequence (e.g., DNA sequence) of WRN. In some embodiments, the agent that reduces the level and/or activity of WRN includes a TALEN, or an mRNA encoding a TALEN, that targets (e.g., cleaves) a nucleic acid sequence (e.g., DNA sequence) of WRN.

For example, the gRNA can be used in a CRISPR system to engineer an alteration in a gene (e.g., WRN). In other examples, the ZFN and/or TALEN can be used to engineer an alteration in a gene (e.g., WRN). Exemplary alterations include insertions, deletions (e.g., knockouts), translocations, inversions, single point mutations, and other mutations. The alteration can be introduced in the gene in a cell. In some embodiments, the alteration decreases the level and/or activity of (e.g., knocks down or knocks out) WRN, e.g., the alteration is a negative regulator of function.

In certain embodiments, the CRISPR system is used to edit (e.g., to add or delete a base pair) a target gene, e.g., WRN. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In embodiments, the CRISPR system is used to direct Cas to a promoter of a target gene, e.g., WRN, thereby blocking an RNA polymerase sterically.

In some embodiments, a CRISPR system can be generated to edit WRN using technology described in, e.g., U.S. Publication No. 20140068797; Cong et al., Science 339(6121):819-823 (2013); Tsai, Nature Biotechnol., 32(6):569-576 (2014); and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., the gene encoding WRN. In CRISPRi, an engineered Cas9 protein (e.g., nuclease-null dCas9, or dCas9 fusion protein, e.g., dCas9-KRAB or dCas9-SID4X fusion) can pair with a sequence-specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some embodiments, CRISPR-mediated gene activation (CRISPRa) can be used for transcriptional activation, e.g., of one or more genes described herein, e.g., a gene that inhibits WRN. In the CRISPRa technique, dCas9 fusion proteins recruit transcriptional activators. For example, dCas9 can be used to recruit polypeptides (e.g., activation domains), such as VP64, or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains), such as VP64. In some examples, the synergistic activation mediator (SAM) system can be used for transcriptional activation. In SAM, MS2 aptamers are added to the sgRNA. MS2 recruits the MS2 coat protein fused to p65AD and heat shock factor 1. The CRISPRi and CRISPRa techniques are described in greater detail, e.g., in Dominguez et al., Nat. Rev. Mol. Cell Biol. 17(1):5-15 (2016), incorporated herein by reference.

TABLE 1
Exemplary Inhibitory Polynucleotides

| | Type of | |
|---|---|---|
| | Interfering | |
| SEQ ID NO. | Polynucleotide | Nucleic Acid Sequence |
| 5 | CRISPR gRNA | GTAAATTGGAAAACCCACGG |
| | (+ strand) | |
| 6 | CRISPR gRNA | ATCCTGTGGAACATACCATG |
| | (+ strand) | |
| 7 | CRISPR gRNA | GTAGCAGTAAGTGCAACGAT |
| | (− strand) | |
| 8 | CRISPR gRNA | CATTACGTATCTCAGTAAGA |
| | (− strand) | |
| 9 | CRISPR gRNA | GGACATGGAATCATATTATC |
| | (+ strand) | |
| 10 | CRISPR gRNA | GAGCACCGTAATGAATGACT |
| | (− strand) | |
| 11 | miRNA | UUCUUCAAAUCACAGUGAACUC |
| 12 | miRNA | UAAUAUUCACAUUGGUGAUGUU |
| 13 | miRNA | UUACAUUUAGAACACGGUUUUU |
| 14 | miRNA | GCACAUAAGAACCUCGUAACUC |
| 15 | miRNA | UUCUUACACCAAGUCUACUGUA |
| 16 | miRNA | UAUACACUCCGCUAGUGCAGUG |
| 17 | miRNA | UUCUGUUACCAGUUGCACUCCC |
| 18 | miRNA | UAUUUAAUCCGCCGCUAGGUGC |
| 19 | miRNA | CUAUAUCAGUACAGUGCCCUGU |
| 20 | miRNA | AAAAUAUACGGUUGCCCUGUGC |
| 21 | siRNA (guide | CCATGAAGACATTGTTTTT |
| | strand) | |
| 22 | siRNA (guide | GCCTTAACAGTCTGGTTAA |
| | strand) | |
| 23 | siRNA (guide | GCACTAGGCAAGTGTATAT |
| | strand) | |
| 24 | siRNA (guide | GGATGAATGTGCAGAATAA |
| | strand) | |
| 25 | siRNA (guide | GACAGATGTTGCCAATAAA |
| | strand) | |
| 26 | siRNA (guide | CAGATATTGTTTTGTATTT |
| | strand) | |
| 27 | siRNA (guide | CATGGAGTGGCCACCATTA |
| | strand) | |
| 28 | siRNA (guide | CTTTCAGTAAATTGGAAAA |
| | strand) | |
| 29 | siRNA (guide | GGAAAAAACATGTAAAAAA |
| | strand) | |
| 30 | siRNA (guide | GTAATAAAACAATGTTTTT |
| | strand) | |
| 31 | siRNA (guide | GCAAAGGTTTGCTATAAAT |
| | strand) | |
| 32 | siRNA (guide | GGGTTTCTATCTTACTAAA |
| | strand) | |
| 33 | siRNA (guide | GAATAAAGTTTGCTGATTT |
| | strand) | |
| 34 | siRNA (guide | GAAAAAACATGTAAAAAAT |
| | strand) | |
| 35 | siRNA (guide | CTATTATTTTTCAAGTAAT |
| | strand) | |
| 36 | siRNA (guide | CCCATGAAGACATTGTTTT |
| | strand) | |
| 37 | siRNA (guide | GCTTATGCTGGTTTTATTA |
| | strand) | |
| 38 | siRNA (guide | CAAAGATGAGTGAAAAAAA |
| | strand) | |
| 39 | siRNA (guide | GTAAGAAAGCATAGTTATT |
| | strand) | |
| 40 | siRNA (guide | CTTCTACGTGACTTTGATA |
| | strand) | |
| 41 | shRNA (loop | GAAGATATTAGCATGAGTCTATCAAGAGTAGACTCATGCTAATAT |
| | bolded) | CTTC |
| 42 | shRNA (loop | GTGGAAACTTCTACGTGACTTTCAAGAGAAGTCACGTAGAAGTT |
| | bolded) | TCCAC |
| 43 | shRNA (loop | GAGACCTGGAGCCTTAACAGTTCAAGAGACTGTTAAGGCTCCA |
| | bolded) | GGTCTC |
| 44 | shRNA (loop | GCAACTGGATATGGAAAGAGTTCAAGAGACTCTTTCCATATCCA |
| | bolded) | GTTGC |
| 45 | shRNA (loop | GTTCCAATCGTTGCACTTACTTCAAGAGAGTAAGTGCAACGATT |
| | bolded) | GGAAC |
| 46 | shRNA (loop | GACATTGTACGTTGCTTAAATTCAAGAGATTTAAGCAACGTACAA |
| | bolded) | TGTC |
| 47 | shRNA (loop | GCACCAAAGAGCATTGTTATATCAAGAGTATAACAATGCTCTTTG |
| | bolded) | GTGC |
| 48 | shRNA (loop | GTAGGCATCAATACTGAGACTTCAAGAGAGTCTCAGTATTGATG |
| | bolded) | CCTAC |
| 49 | shRNA (loop | GTTTCGAAGATCCGTATAACTTCAAGAGAGTTATACGGATCTTC |
| | bolded) | GAAAC |
| 50 | shRNA (loop | GTATGTTCTGGATGACTAATATCAAGAGTATTAGTCATCCAGAAC |
| | bolded) | ATAC |

Small Molecule Compounds

In some embodiments of the invention, the agent that reduces the level and/or activity of WRN in a cell in a subject is a small molecule compound. Small molecules compounds include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically-acceptable forms of such compounds.

Antibodies

The agent that reduces the level and/or activity of WRN in a cell in a subject can be an antibody or antigen binding fragment thereof. Antibodies and antigen-binding fragments, variants, or derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments (e.g., Fab, Fab′ and F(ab′)2), Fd, Fvs, single-chain Fvs (scFv), rIgG, single-chain antibodies, disulfide-linked Fvs (sdFv), fragments including either a VL or VH domain, fragments produced by an Fab expression library, nanobodies, affibodies, aptamers, small molecule immunopharmaceuticals (SMIPs), and anti-idiotypic (anti-Id) antibodies. Antibody molecules can be of any type (e.g., IgG, IgE, IgM, IgD, IgA, and IgY), class (e.g., IgG1, IgG2, IgG3, IgG4, IgA1 and IgA2) or subclass of immunoglobulin molecule. For example, an agent that reduces the level and/or activity of WRN described herein is an antibody (e.g., a polyclonal, monoclonal, humanized, chimeric, or heteroconjugate antibody), or an antigen-binding fragment thereof (e.g., a Fab (e.g., a F(ab′)2), scFv, SMIP, diabody, a triabody, an affibody, a nanobody, an aptamer, or a single domain antibody) that reduces or blocks the activity and/or function of WRN through binding to WRN.

The making and use of therapeutic antibodies, and antigen-binding fragments thereof against a target antigen (e.g., WRN) is known in the art. Antibodies and antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art. See, for example, the references cited herein above, as well as Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

### Pharmaceutical Uses

The agents that reduce the level and/or activity of WRN in a cell in a subject as described herein are useful in the methods of the invention and, while not bound by theory, are believed to exert their desirable effects through their ability to modulate the level, status, and/or activity of WRN, e.g., by inhibiting the activity or level of WRN in a cell in a mammal.

An aspect of the present invention relates to methods of treating a cancer having a mutation that results in a loss of function of ARID1A in a subject in need thereof. In some embodiments, the method includes administering to the subject an effective amount of an agent that reduces the level and/or activity of WRN in a cell in the subject. In some embodiments, the WRN activity is WRN helicase activity. In some embodiments, the agent that reduces the level and/or activity of WRN in a cell in a subject is administered in an amount and for a time effective to result in one (or more, e.g., two or more, three or more, four or more of) of: (a) reduced tumor size, (b) reduced rate of tumor growth, (c) increased tumor cell death, (d) reduced tumor progression, (e) reduced number of metastases, (f) reduced rate of metastasis, (g) decreased tumor recurrence, (h) increased survival of subject, and (i) increased progression free survival of a subject.

Another aspect of the present invention relates to methods of treating a cancer having a MMRd in a subject in need thereof. In some embodiments, the method includes administering to the subject an effective amount of an agent that reduces the level and/or activity of WRN in a cell in the subject. In some embodiments, the WRN activity is WRN helicase activity. In some embodiments, the agent that reduces the level and/or activity of WRN in a cell in a subject is administered in an amount and for a time effective to result in one (or more, e.g., two or more, three or more, four or more of) of: (a) reduced tumor size, (b) reduced rate of tumor growth, (c) increased tumor cell death, (d) reduced tumor progression, (e) reduced number of metastases, (f) reduced rate of metastasis, (g) decreased tumor recurrence, (h) increased survival of subject, and (i) increased progression free survival of a subject.

Another aspect of the present invention relates to methods of treating a cancer having an MSI-positive phenotype in a subject in need thereof. In some embodiments, the method includes administering to the subject an effective amount of an agent that reduces the level and/or activity of WRN in a cell in the subject. In some embodiments, the WRN activity is WRN helicase activity. In some embodiments, the agent that reduces the level and/or activity of WRN in a cell in a subject is administered in an amount and for a time effective to result in one (or more, e.g., two or more, three or more, four or more of) of: (a) reduced tumor size, (b) reduced rate of tumor growth, (c) increased tumor cell death, (d) reduced tumor progression, (e) reduced number of metastases, (f) reduced rate of metastasis, (g) decreased tumor recurrence, (h) increased survival of subject, and (i) increased progression free survival of a subject.

Another aspect of the present invention relates to methods of treating a cancer having an MSI-high (MSI-H) phenotype in a subject in need thereof. In some embodiments, the method includes administering to the subject an effective amount of an agent that reduces the level and/or activity of WRN in a cell in the subject. In some embodiments, the WRN activity is WRN helicase activity. In some embodiments, the agent that reduces the level and/or activity of WRN in a cell in a subject is administered in an amount and for a time effective to result in one (or more, e.g., two or more, three or more, four or more of) of: (a) reduced tumor size, (b) reduced rate of tumor growth, (c) increased tumor cell death, (d) reduced tumor progression, (e) reduced number of metastases, (f) reduced rate of metastasis, (g) decreased tumor recurrence, (h) increased survival of subject, and (i) increased progression free survival of a subject.

Treating cancer with an effective amount of an agent that reduces the level and/or activity of WRN in a cell in the subject may further result in an increase in double-strand breaks within the cell and/or alteration of the cell cycle of the cell in the subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the anti-WRN agent described herein. An increase in average survival time of a population may also be measured, for example, by calculating for a population the average length of survival following completion of a first round of treatment with an anti-WRN agent described herein.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with an anti-WRN agent described herein. A decrease in the mortality rate of a population may also be measured, for example, by calculating for a population the average number of disease-related deaths per unit time following completion of a first round of treatment with an anti-WRN agent as described herein.

Selection of Subjects

Subjects that may be treated using the methods described herein are subjects having a cancer characterized by a mutation that results in a loss of function of ARID1A. In some embodiments, the cancer has a MMRd. In some embodiments, the MMRd is caused by a mutation in the MLH1, MLH3, MSH2, MSH3, MSH6, PMS1, PMS2, and/or EPCAM genes. In some embodiments, the MMRd is caused by a mutation in the MLH1, MSH2, MSH6, PMS2, and/or EPCAM genes. In some embodiments, the MMRd is caused by a mutation in the MLH1 gene. In some embodiments, the mutation of the MLH1 gene results in a reduction or a loss of function of MLH1. In some embodiments, the cancer has an MSI-positive phenotype characterized by the presence of an MSI at least one of the mononucleotide or dinucleotide markers selected from the group consisting of BAT25, BAT26, D2S123, D5S346, and D175250. In some embodiments, the MSI-positive phenotype is an MSI-high (MSI-H) phenotype characterized by the presence of an MSI at least two of the mononucleotide or dinucleotide markers selected from the group consisting of BAT25, BAT26, D2S123, D5S346, and D17S250.

Subjects that may be treated using the methods described herein are subjects having a cancer characterized by a MMRd. In some embodiments, the MMRd is caused by a mutation in the MLH1, MLH3, MSH2, MSH3, MSH6, PMS1, PMS2, and/or EPCAM genes. In some embodiments, the MMRd is caused by a mutation in the MLH1, MSH2, MSH6, PMS2, and/or EPCAM genes. In some embodiments, the MMRd is caused by a mutation in the MLH1 gene. In some embodiments, the mutation of the MLH1 gene results in a reduction or a loss of function of MLH1. In some embodiments, the cancer has an MSI-positive phenotype characterized by the presence of an MSI at least one of the mononucleotide or dinucleotide markers selected from the group consisting of BAT25, BAT26, D2S123, D5S346, and D17S250. In some embodiments, the MSI-positive phenotype is an MSI-high (MSI-H) phenotype characterized by the presence of an MSI at least two of the mononucleotide or dinucleotide markers selected from the group consisting of BAT25, BAT26, D2S123, D5S346, and D17S250. In some embodiments, the cancer additionally has a mutation that results in a loss of function of ARID1A.

Subjects that may be treated using the methods described herein are subjects having a cancer characterized by an MSI-positive phenotype. In some embodiments, the MSI-positive phenotype characterized by the presence of an MSI at least one of the mononucleotide or dinucleotide markers selected from the group consisting of BAT25, BAT26, D2S123, D5S346, and D17S250. In some embodiments, the MSI-positive phenotype is an MSI-high (MSI-H) phenotype characterized by the presence of an MSI at least two of the mononucleotide or dinucleotide markers selected from the group consisting of BAT25, BAT26, D2S123, D5S346, and D175250. In some embodiments, the cancer has a MMRd. In some embodiments, the MMRd is caused by a mutation in the MLH1, MLH3, MSH2, MSH3, MSH6, PMS1, PMS2, and/or EPCAM genes. In some embodiments, the MMRd is caused by a mutation in the MLH1, MSH2, MSH6, PMS2, and/or EPCAM genes. In some embodiments, the MMRd is caused by a mutation in the MLH1 gene. In some embodiments, the cancer additionally has a mutation that results in a loss of function of ARID1A.

The types of cancer may include, for example, an MSI-positive cancer, an MSI-H cancer, adrenocortical carcinoma, bladder carcinoma, breast carcinoma, cervical squamous cell carcinoma, endocervical adenocarcinoma, cholangiocarcinoma, chronic lymphocytic leukemia, a colorectal cancer, colon adenocarcinoma, an ovarian cancer, cutaneous T-cell lymphoma, lymphoid neoplasm diffuse large B-cell lymphoma, esophageal carcinoma, glioblastoma multiforme, head and neck squamous cell carcinoma, kidney chromophobe, kidney renal papillary cell carcinoma, acute myeloid leukemia, lower-grade glioma, liver hepatocellular carcinoma, lung adenocarcinoma, lung squamous cell carcinoma, mesothelioma, nasopharyngeal carcinoma, ovarian serous cystadenocarcinoma, pancreatic adenocarcinoma, pheochromocytoma, paraganglioma, prostate adenocarcinoma, rectal adenocarcinoma, sarcoma, skin cutaneous melanoma, stomach adenocarcinoma, testicular germ cell tumor, thyroid carcinoma, thymoma, uterine corpus endometrial carcinoma, uterine carcinosarcoma, uveal melanoma, pediatric acute myeloid leukemia, pediatric neuroblastoma, pediatric high-risk Wilms tumor, or any other type of cancer as described herein. The cancer may be of early or advanced stage (e.g., a recurrent or metastatic cancer). In some embodiments, the subject has received prior anti-cancer therapy. In some embodiments, the subject has not been previously treated with an anti-cancer therapy. In some embodiments, the cancer is resistant to immunotherapy (e.g., a checkpoint inhibitor as described herein). In some embodiments, the cancer is resistant to targeted therapy. In some embodiments, the therapeutic resistance is driven by the deficiency in MMR, such as resistance to endocrine treatment in breast cancers and resistance to targeted therapy (e.g., temozolomide) in glioblastomas.

Combination Therapies

An agent that reduces the level and/or activity of WRN in a cell in a subject as described herein, can be administered alone or in combination with an additional anti-cancer therapy. The anti-cancer therapy may be an additional therapeutic agent (e.g., other agents that treat cancer or symptoms associated therewith) or in combination with other types of therapies to treat cancer (e.g., radiological therapies or surgical procedures). In some embodiments, the second therapeutic agent is selected based on tumor type, tumor tissue of origin, tumor stage, or mutation status. In combination treatments, the dosages of one or more of the therapeutic agents may be reduced from standard dosages when administered alone. For example, doses may be determined empirically from drug combinations and permutations or may be deduced by isobolographic analysis (e.g., Black et al., Neurology 65:S3-S6 (2005)). In this case, dosages of the agents or compounds when combined should provide a therapeutic effect.

In some embodiments, the anti-cancer therapy is a checkpoint inhibitor. In some embodiments, the inhibitor of checkpoint is an inhibitory antibody (e.g., a monospecific antibody, such as a monoclonal antibody). The antibody may be humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody or a fusion protein, such as ipilimumab/YERVOY® or tremelimumab). In some embodiments, the inhibitor of checkpoint is an inhibitor (e.g., an inhibitory antibody or small molecule inhibitor) of PD-1 (e.g., nivolumab/OPDIVO®; pembrolizumab/KEYTRUDA®; or pidilizumab/CT-011). In some embodiments, the inhibitor of checkpoint is an inhibitor (e.g., an inhibitory antibody or small molecule inhibitor) of PDL1 (e.g., MPDL3280A/RG7446/atezolizumab; MED14736/durvalumab; MSB0010718C/avelumab; BMS 936559/cemiplimab). In some embodiments, the inhibitor of checkpoint is an inhibitor (e.g., an inhibitory antibody or Fc fusion or small molecule inhibitor) of PDL2 (e.g., a PDL2/Ig fusion protein, such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAGS, VISTA, KIR, 2B4, CD160, CGEN-15049, CHK 1, CHK2, A2aR, B-7 family ligands, or a combination thereof.

In some embodiments, the anti-cancer therapy is a biologic, such as a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (Rituximab); ZENAPAX® (Daclizumab); SIMULECT® (Basiliximab); SYNAGIS® (Palivizumab); REMICADE® (Infliximab); HERCEPTIN® (Trastuzumab); MYLOTARG™ (Gemtuzumab ozogamicin); CAMPATH® (Alemtuzumab); ZEVALIN® (Ibritumomab tiuxetan); HUMIRA® (Adalimumab); XOLAIR® (Omalizumab); BEXXAR® (Tositumomab-I-131); RAPTIVA® (Efalizumab); ERBITUX® (Cetuximab); AVASTIN® (Bevacizumab); TYSABRI® (Natalizumab); ACTEMRA® (Tocilizumab); VECTIBIX® (Panitumumab); LUCENTIS® (Ranibizumab); SOLIRIS® (Eculizumab); CIMZIA® (Certolizumab pegol); SIMPONI® (Golimumab); ILARIS® (Canakinumab); STELARA® (Ustekinumab); ARZERRA® (Ofatumumab); PROLIA® (Denosumab); Numax (Motavizumab); ABThrax (Raxibacumab); BENLYSTA® (Belimumab); YERVOY® (Ipilimumab); ADCETRIS® (Brentuximab Vedotin); PERJETA® (Pertuzumab); KADCYLA® (Ado-trastuzumab emtansine); and GAZYVA® (Obinutuzumab). Also included are antibody-drug conjugates.

In some embodiments, the anti-cancer therapy is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin, irenotecan, oxaliplatin, capecitabine, paclitaxel, and doxetaxel. Non-limiting examples of chemotherapeutic agents include alkylating agents, such as thiotepa and cyclosphosphamide; alkyl sulfonates, such as busulfan, improsulfan and piposulfan; aziridines, such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, such as methotrexate and 5-fluorouracil; folic acid analogues, such as denopterin, pteropterin, trimetrexate; purine analogs, such as fludarabine, 6-mercaptopurine, thiamiprine, thioguanine; pyrimidine analogs, such as ancitabine, azacitidine, 6-azauridine, carmofur, cytarabine, dideoxyuridine, doxifluridine, enocitabine, floxuridine; androgens, such as calusterone, dromostanolone propionate, epitiostanol, mepitiostane, testolactone; anti-adrenals, such as aminoglutethimide, mitotane, trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, e.g., TAXOL® (paclitaxel; Bristol-Myers Squibb Oncology, Princeton, N.J.), ABRAXANE®, cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; platinum coordination complexes, such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; XELODA®; ibandronate; irinotecan (e.g., CPT-11); topoisomerase inhibitor RFS 2000; difluoromethylornithine (DMFO); retinoids, such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc. Am. Soc. Clin. Oncol. 18:233a (1999), and Douillard et al., Lancet 355(9209):1041-1047 (2000).

In some embodiments, the anti-cancer therapy is a T cell adoptive transfer therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

The additional anti-cancer therapy may be a non-drug treatment. For example, the additional therapeutic agent is radiation therapy, cryotherapy, hyperthermia, and/or surgical excision of tumor tissue.

In any of the combination embodiments described herein, the agent that reduces the level and/or activity of WRN in a cell in a subject and additional therapeutic agents are administered simultaneously or sequentially, in either order. The agent that reduces the level and/or activity of WRN in a cell in a subject may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours, or up to 1-7, 1-14, 1-21, or 1-30 days before or after the additional therapeutic agent (e.g., an anti-cancer therapy).

Delivery of anti-WRN Agents

A variety of methods are available for the delivery of anti-WRN agents to a subject including viral and non-viral methods.

Viral Delivery Methods

In some embodiments, the agent that reduces the level and/or activity of WRN in a cell in a subject is delivered by a viral vector (e.g., a viral vector expressing an anti-WRN agent, such as a polynucleotide as described herein). Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative-strand RNA viruses, such as orthomyxovirus (e.g., influenza virus), rhabdovirus (e.g., rabies and vesicular stomatitis virus), paramyxovirus (e.g., measles and Sendai), positive-strand RNA viruses, such as picornavirus and alphavirus, and double-stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara, fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference.

Exemplary viral vectors include lentiviral vectors, AAVs, and retroviral vectors. Lentiviral vectors and AAVs can integrate into the genome without cell divisions, and both types have been tested in pre-clinical animal studies. Methods for preparation of AAVs are described in the art e.g., in U.S. Pat. Nos. 5,677,158, 6,309,634, and 6,683,058, each of which is incorporated herein by reference. Methods for preparation and in vivo administration of lentiviruses are described in US 20020037281 (incorporated herein by reference). Preferably, a lentiviral vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding the fusion protein, an origin of second strand DNA synthesis and a 3′ lentiviral LTR.

Retroviruses are most commonly used in human clinical trials, as they carry 7-8 kb, and have the ability to infect cells and have their genetic material stably integrated into the host cell with high efficiency (see, e.g., WO 95/30761; WO 95/24929, each of which is incorporated herein by reference). Preferably, a retroviral vector is replication defective. This prevents further generation of infectious retroviral particles in the target tissue. Thus, the replication defective virus becomes a “captive” transgene stable incorporated into the target cell genome. This is typically accomplished by deleting the gag, env, and pol genes (along with most of the rest of the viral genome). Heterologous nucleic acids are inserted in place of the deleted viral genes. The heterologous genes may be under the control of the endogenous heterologous promoter, another heterologous promoter active in the target cell, or the retroviral 5′ LTR (the viral LTR is active in diverse tissues).

These delivery vectors described herein can be made target-specific by attaching, for example, a sugar, a glycolipid, or a protein (e.g., an antibody to a target cell receptor).

Reversible delivery expression systems may also be used. The Cre-loxP or FLP/FRT system and other similar systems can be used for reversible delivery-expression of one or more of the above-described nucleic acids. See WO2005/112620, WO2005/039643, US20050130919, US20030022375, US20020022018, US20030027335, and US20040216178. In particular, the reversible delivery-expression system described in US20100284990 can be used to provide a selective or emergency shut-off.

Non-Viral Delivery Methods

Several non-viral methods exist for delivery of anti-WRN agents including polymeric, biodegradable microparticle, or microcapsule delivery devices known in the art. For example, a colloidal dispersion system may be used for targeted delivery an anti-WRN agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles, which range in size from 0.2-4.0 μm can encapsulate a substantial percentage of an aqueous buffer containing large macromolecules.

The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Lipids useful in liposome production include phosphatidyl compounds, such as phosphatidylglycerol, phosphatidylcholine, phosphatidylserine, phosphatidyl-ethanolamine, sphingolipids, cerebrosides, and gangliosides. Phospholipids include egg phosphatidylcholine, dipalmitoylphosphatidylcholine, and distearoyl-phosphatidylcholine. The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255.

Pharmaceutical Compositions and Routes of Administration

Anti-WRN agents for use in the methods described herein may be placed into a pharmaceutically-acceptable suspension, solution, or emulsion.

The anti-WRN agents described herein may be administered, for example, by parenteral, intratumoral, oral, buccal, sublingual, nasal, rectal, patch, pump, or transdermal administration. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

In some embodiments, an anti-WRN agent for use in the methods described herein is administered intratumorally, for example, as an intratumoral injection. Intratumoral injection is injection directly into the tumor vasculature and is specifically contemplated for discrete, solid, accessible tumors. Local, regional, or systemic administration also may be appropriate. An anti-WRN agent described herein may advantageously be contacted by administering an injection or multiple injections to the tumor, spaced for example, at approximately, 1 cm intervals. In the case of surgical intervention, anti-WRN agents may be used preoperatively, such as to render an inoperable tumor subject to resection. Continuous administration also may be applied where appropriate, for example, by implanting a catheter into a tumor or into tumor vasculature.

In some embodiments, an anti-WRN agent described herein is administered parenterally (e.g., intravenously). Solutions of an anti-WRN agent described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

An anti-WRN agent described herein may be orally administered, for example, with an inert diluent or with an assimilable edible carrier, may be enclosed in hard or soft shell gelatin capsules, may be compressed into tablets, or may be incorporated directly with the food of the diet. For oral therapeutic administration, an anti-WRN agent described herein may be incorporated with an excipient and used in the form of ingestible tablets, buccal tablets, troches, capsules, elixirs, suspensions, syrups, and wafers. An anti-WRN agent described herein formulated for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single-dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. An anti-WRN agent described herein formulated for buccal or sublingual administration include tablets, lozenges, and pastilles, where the active ingredient is formulated with a carrier, such as sugar, acacia, tragacanth, gelatin, and glycerine. An anti-WRN agent described herein formulated for rectal administration are conveniently in the form of suppositories containing a conventional suppository base, such as cocoa butter.

Dosing

The dosage of the anti-WRN agents described herein, and/or compositions including an anti-WRN agent described herein, can vary depending on many factors, such as the pharmacodynamic properties of the agent or compound; the mode of administration; the age, health, and weight of the recipient; the nature and extent of the symptoms; the frequency of the treatment, and the type of concurrent treatment, if any; and the clearance rate of the agent or compound in the animal to be treated. One of skill in the art can determine the appropriate dosage based on the above factors. The anti-WRN agents described herein may be administered initially in a suitable dosage that may be adjusted as required, depending on the clinical response.

### Kits

The invention also features kits including (a) a pharmaceutical composition including an agent that reduces the level and/or activity of WRN in a cell described herein, and (b) a package insert with instructions to perform any of the methods described herein. In some embodiments, the kit includes (a) a pharmaceutical composition including an agent that reduces the level and/or activity of WRN in a cell described herein, (b) an additional therapeutic agent (e.g., an anti-cancer agent), and (c) a package insert with instructions to perform any of the methods described herein.

### EXAMPLES

### Example 1: WRN Protein Depletion Results in Strong Growth Inhibition of Cancer Cells with Mutated ARID1A, but not with Wild Type ARID1A

The following example demonstrates that the depletion of the WRN protein results in strong growth inhibition in cancer cells with ARID1A loss.

Procedure: Indicated cell lines expressing Cas9 were generated by lentiviral transduction of the Cellecta-pR-CMV-Cas9-2A-Blast vector. Positive populations were selected using Blasticidin S (Thermo Scientific). Individual sgRNAs targeting WRN were cloned into a doxycycline (Dox)-inducible U6 promoter sgRNA-expressing vector. Cas9 stable cells were infected with lentiviral vectors expressing the inducible sgRNAs. Positive populations were selected using puromycin (Thermo Scientific). Cells expressing both Cas9 and sgRNAs were seeded into 6-well plates with or without Dox. Western samples were collected at day 5 of Dox treatment. Colony formation samples were fixed and stained with crystal violet after 10-14 days of Dox treatment.

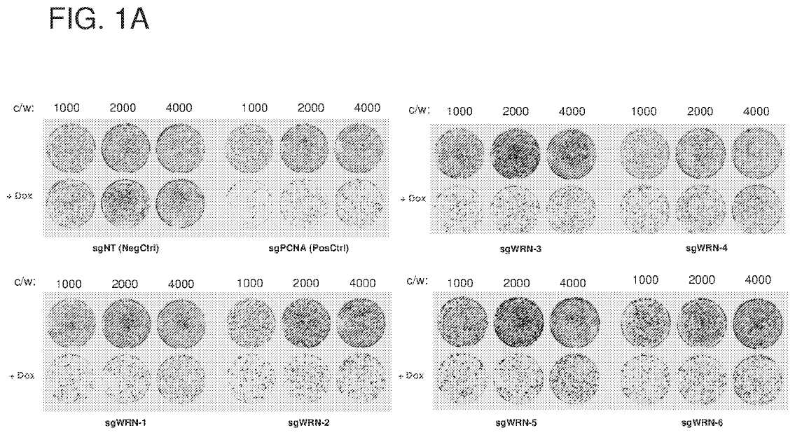

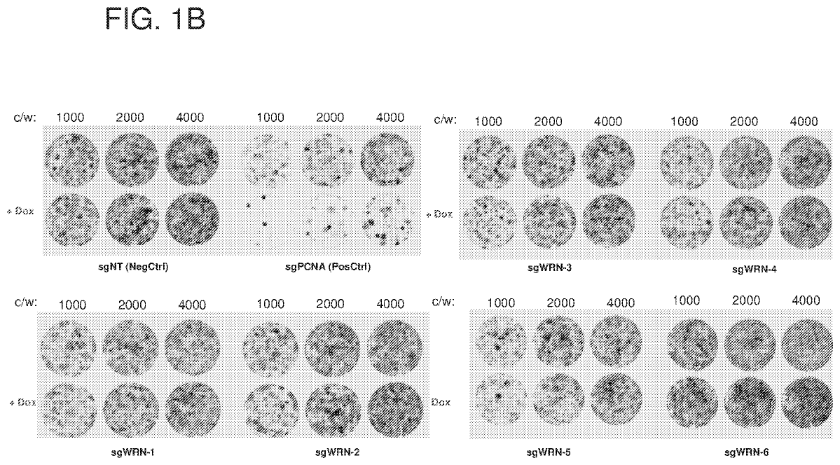

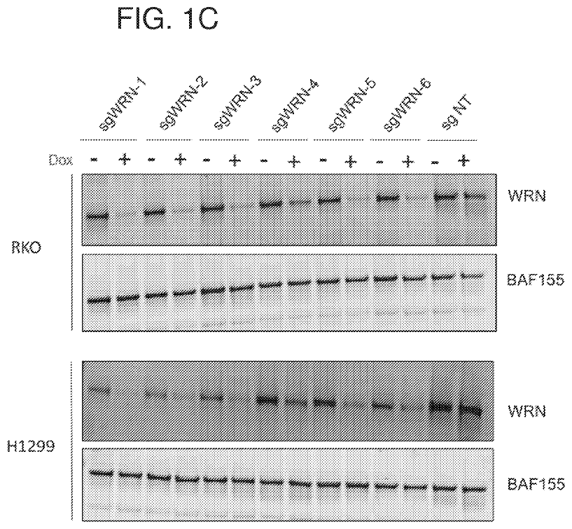

Results: Depletion of WRN using inducible CRISPR in an ARID1A-mutant cell line, RKO, resulted in strong growth inhibition (FIGS. 1A and 1C), demonstrating synthetic lethality of WRN and ARID1A. Depletion of WRN in an ARID1A-wild type cell line, NCI-H1299, did not affect cell proliferation (FIGS. 1B and 1C).

### Example 2: WRN-Sensitive Cell Lines Exhibit Typical Microsatellite Instability Mutation Signature (MSI+)

The following example identifies an MSI mutation signature for cells sensitive to WRN inhibition.

Procedure: For each cell line, all mutations were compiled from the Cancer Cell Line Encyclopedia (CCLE) and mutation context was extracted. For each cell line, the mutation context profile was compared to the Catalog of Somatic Mutations in Cancer (COSMIC) mutational signatures, and Euclidean distances were calculated.

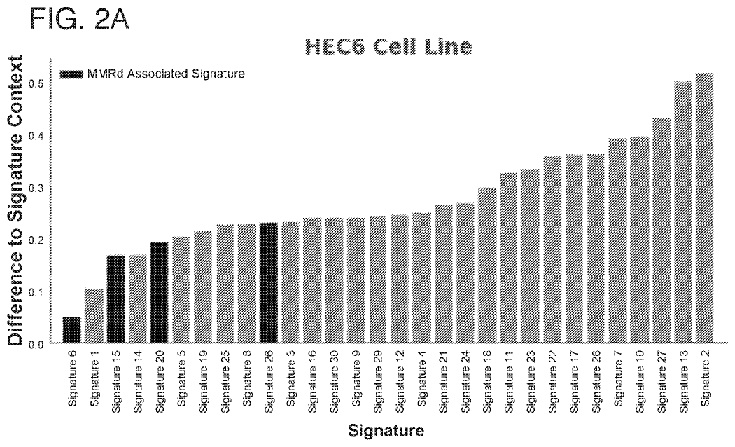

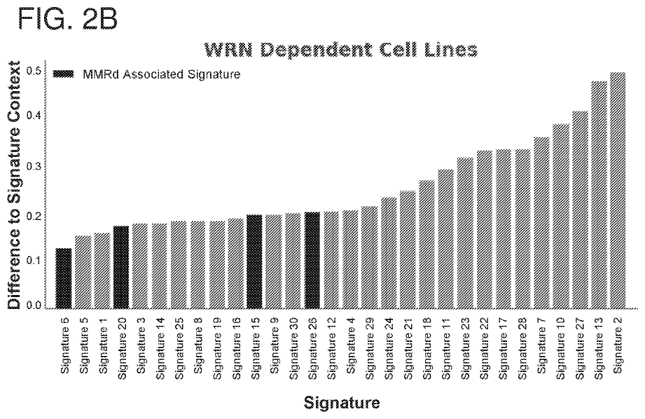

Results: As shown in FIG. 2, the mutation context of a top WRN-dependent cell line, HEC6, shows closest proximity to COSMIC signature 6 (a MMRd associated signature), COSMIC signature 6 is also the most closely related to all WRN-dependent cell lines (classified as described above).

### Example 3: Identification of Tumor Samples Dependent on WRN Protein

The following example identifies tumors with predicted dependence on WRN, and examines additional commonalities among these tumors.

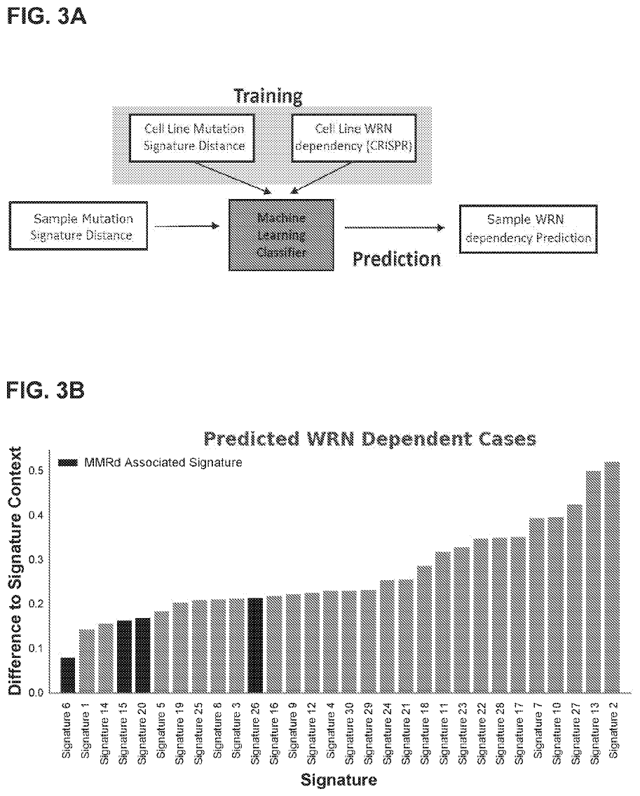

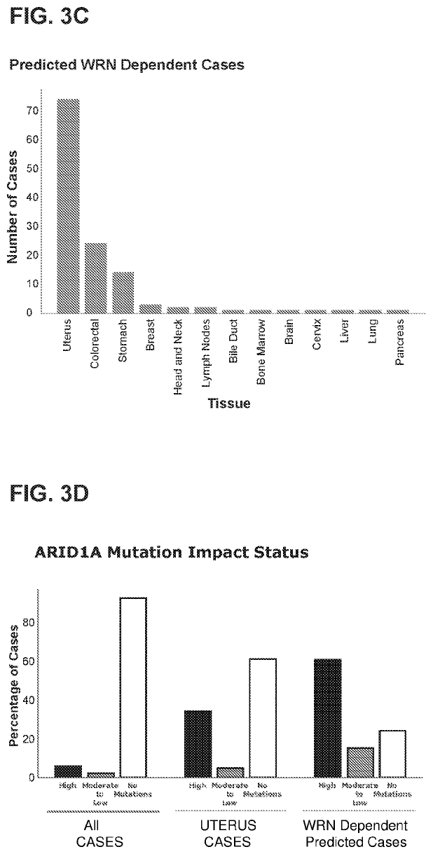

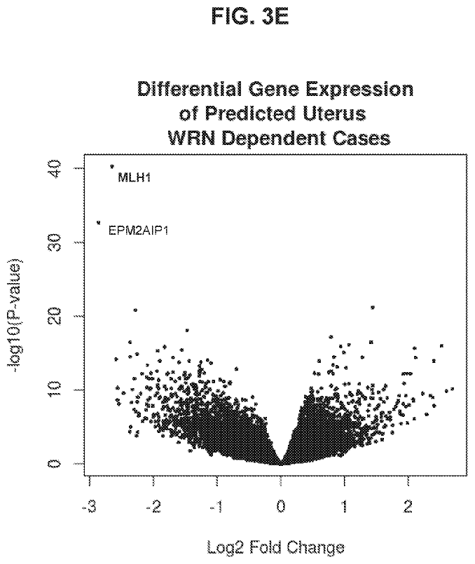

Procedure: A classifier was created based on WRN dependency determined by classifying CRISPR screening effects of WRN depletion on tumor cells using K-means clustering and distance to COSMIC mutation signatures (FIG. 3A). This tool was used to classify tumor samples regarding WRN-dependency using the distances from the COSMIC mutation signatures for each sample (FIG. 3B). Additional mutation status was also examined for all tumors classified.

Results: Uterine, colorectal, and stomach cancers, specifically uterine corpus endometrial carcinoma, colon adenocarcinoma, and stomach adenocarcinoma, were among those tumors assessed that had the highest number of WRN-dependent predicted tumors (FIG. 3C). A high percentage of WRN-dependent predicted tumors also had mutations in ARID1A (FIG. 3D). MLH1 was also found to be most consistently downregulated in WRN-dependent predicted tumors (FIG. 3E).

### Example 4: WRN Protein Depletion Results in Strong Growth Inhibition of Cancer Cells with Mismatch Repair Deficiency

The following example demonstrates that the depletion of the WRN protein results in strong growth inhibition in cancer cells with MMRd.

Procedure: Indicated cell lines expressing Cas9 were generated by lentiviral transduction of the Cellecta-pR-CMV-Cas9-2A-Blast vector. Positive populations were selected using Blasticidin S (Thermo Scientific). Individual sgRNAs targeting WRN were cloned into a Dox-inducible U6 promoter sgRNA-expressing vector. Cas9 stable cells were infected with lentiviral vectors expressing the inducible sgRNAs. Positive populations were selected using puromycin (Thermo Scientific). Cells expressing both Cas9 and sgRNAs were seeded into 6-well plates with or without Dox. Western samples were collected at day 5 of Dox treatment. Colony formation samples were fixed and stained with crystal violet after 10-14 days of Dox treatment.

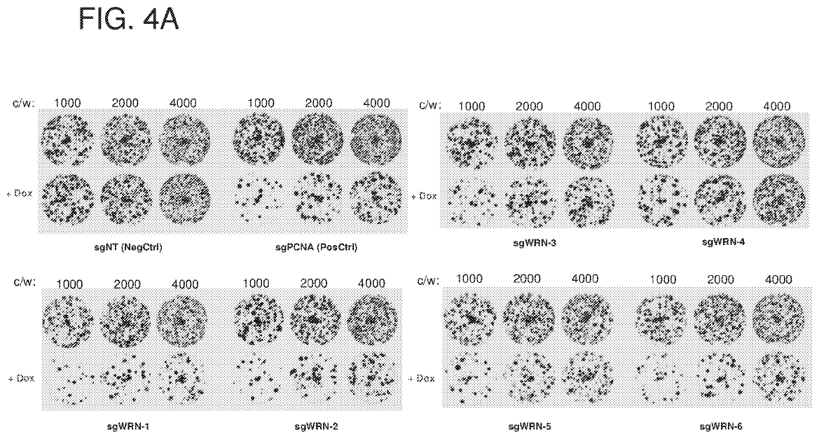

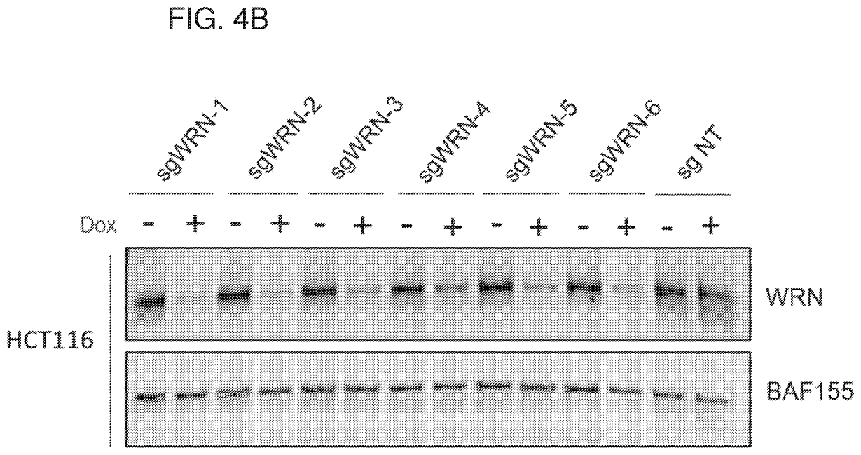

Results: As shown in FIGS. 4A-4B, depletion of WRN using inducible CRISPR in the MMRd cancer cell line HCT116 resulted in strong growth inhibition.

### Example 5: The ATP-Dependent Helicase Function of WRN is Required to Maintain Cancer Cell Survival

The following example demonstrates that the helicase activity of WRN protein is critical for cancer cells with ARID1A loss or MMRd.

Procedure: RKO cells expressing Cas9 and inducible sgWRN-1 were transduced with lentivirus-expressing indicated WRN variants. Stable cells expressing WRN variants were generated after G418 (Thermo Scientific) selection. Cells expressing both Cas9 and sgRNAs, as well as WRN variants, were seeded into 6-well plates with or without Dox. Western samples were collected at day 5 of Dox treatment. Colony formation samples were fixed and stained with crystal violet after 10-14 days of Dox treatment.

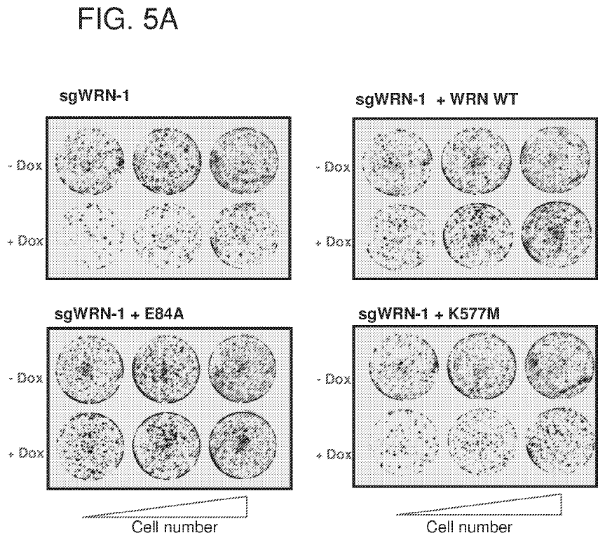

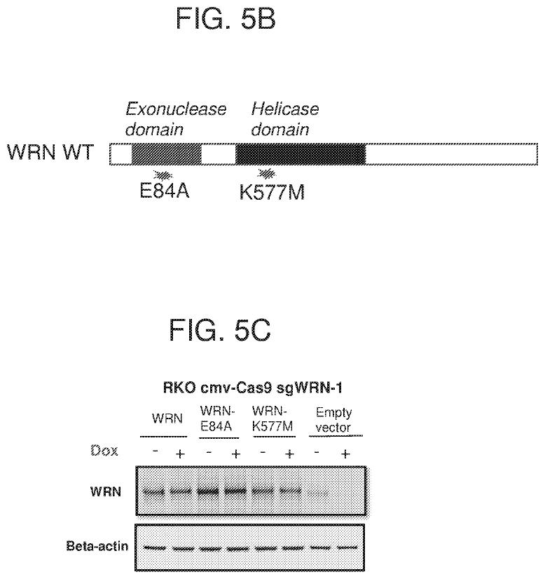

Results: Expressing wild type WRN or the exonuclease domain mutant WRN E84A rescued the growth inhibition caused by the loss of endogenous WRN in RKO cells. However, expressing the helicase domain mutant WRN K577M did not restore growth in these cells (FIGS. 5A-5C). These results indicate that WRN helicase activity is necessary for cancer cell survival.

### Example 6: WRN Depletion in HCT116 (MMRd) and RKO (MMRd; ARID1A-Mutant) Cells Induces DNA Damage Response

The following example demonstrates that depletion of the WRN protein results in a strong DNA damage response in cancers cells with ARID1A mutation and MMRd (RKO is ARID1A mutant and MMRd; HCT116 is ARID1A wt and MMRd).

Procedure: ecDHFR Degron Domain was knocked into endogenous WRN N-terminus to create ecDHFR-WRN in HCT116 cells. WRN protein undergoes degradation in the absence of compound trimethoprim (TMP). HCT116-ecDHFR-WRN cells were treated with indicated concentrations of TMP for 72 hours. SMASh tag was knocked into the endogenous WRN N-terminus to create SMASh-WRN in RKO cells. WRN protein undergoes degradation in the presence of compound asunaprevir (ASV). RKO-SMASh-WRN cells were treated with indicated concentrations of ASV for 72 hours. The results were assessed by immunoblotting using the following detection antibodies: WRN (Cell Signaling Technology (CST) #4666); pH2AX (CST #9718); pCHK2 (CST #2197); CHK2 (CST #6334); p21 (CST #2947); pP53 (CST #16G8); Tubulin (CST #2128). Tubulin was used as a loading control.

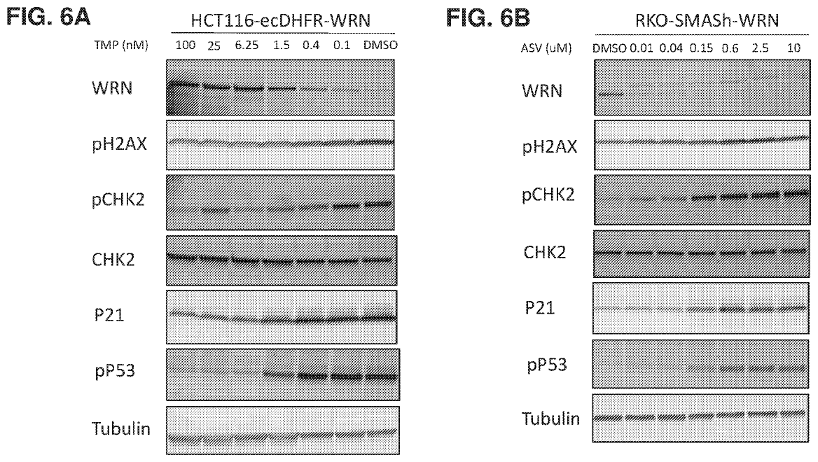

Results: As shown in FIGS. 6A-6B, dose-dependent depletion of WRN protein resulted in dose-dependent induction of the DNA damage response markers pH2AX, pCHK2, pP53, and p21.

### Example 7: WRN Depletion by Inducible CRISPR or SMASh Degron Technology Reduces Tumor Growth in HCT116 and RKO Xenograft Models

The following example demonstrates that depletion of the WRN protein results in tumor growth reduction in HCT116 and RKO xenograft models.

Procedure: HCT116 sgNT (AAGATCGAGTGCCGCATCAC, SEQ ID NO: 51), sgWRN-1 (GTAAATTGGAAAACCCACGG, SEQ ID NO: 5), and sgWRN-2 (ATCCTGTGGAACATACCATG, SEQ ID NO: 6) xenografts were established by subcutaneous inoculation of 5 million cells into 6-8 week old Balb/c Nude female mice. RKO and RKO-SMASh-WRN xenografts were established by subcutaneous inoculation of 10 million cells into 6-8 week old Balb/c Nude female mice. Both doxycycline (Dox) and ASV compound treatment were started when the average tumor size reach around 200 mm³. Each treatment group contained 8 animals. For the HCT116 study, three tumor samples from each group were collected for western blot analysis after 8 days of Dox treatment. For the RKO study, three tumor samples from each group were collected for western blot analysis after 4 days of ASV treatment (7 h post last treatment). Tumor volume was measured twice weekly by calipering in two dimensions and calculated as width²×length×/6. 300 mg/kg ASV was orally administered once daily in a 10% ethanol/90% PEG400 formulation.

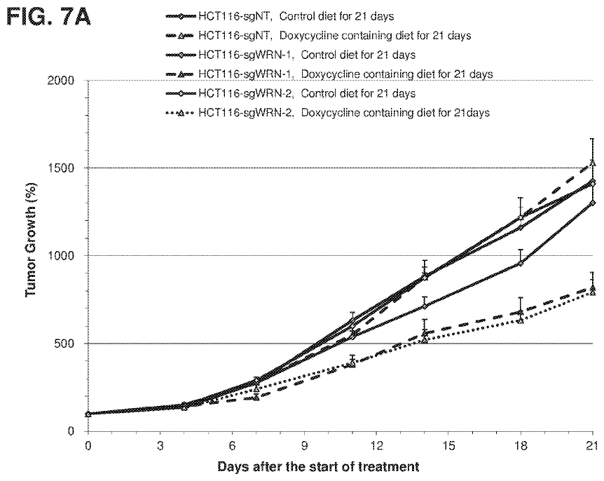

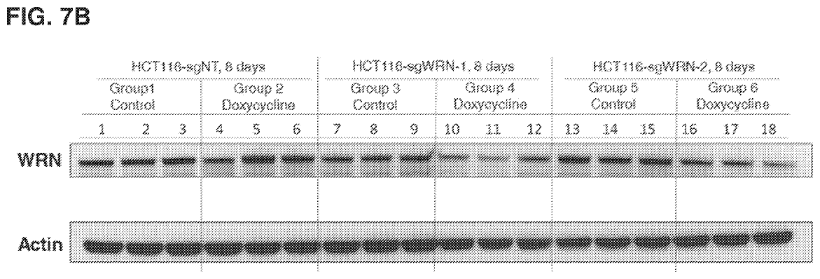

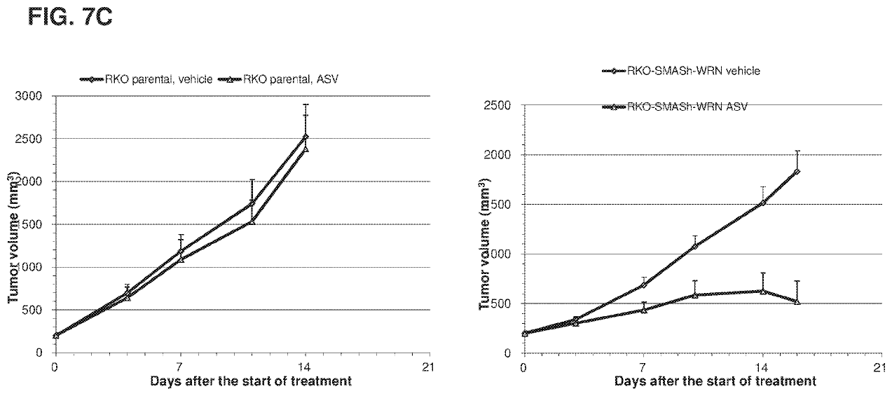

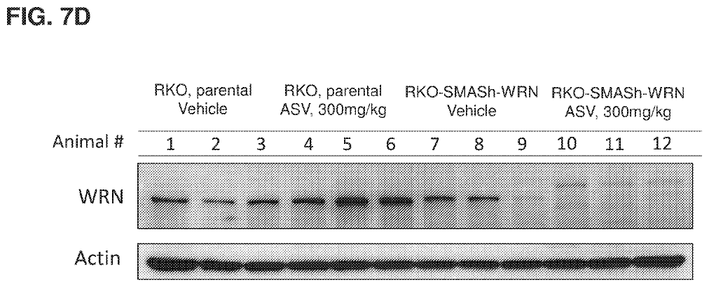

Results: As shown in FIGS. 7A-7D, WRN protein depletion by inducible CRISPR sgWRN-1 and sgWRN-2 resulted in tumor growth reduction. Note that inducible CRISPR only led to partial WRN protein depletion due to the limitation of CRISPR technology. ASV treatment led to WRN protein depletion in RKO-SMASh-WRN tumors, but not RKO parental tumors, as expected. ASV treatment resulted in growth reduction of RKO-SMASh-WRN xenograft model, but not the RKO parental model.

### Example 8: Differential Gene Expression Analysis in RKO Cell Line Following WRN CRISPR Induction

The following example demonstrates that WRN knockdown by CRISPR in RKO cells leads to cell cycle arrest and an activation of p53 DNA damage checkpoint pathway.

Procedure: RKO-sgNT (AAGATCGAGTGCCGCATCAC, SEQ ID NO: 51), sgWRN-1 (GTAAATTGGAAAACCCACGG, SEQ ID NO: 5), or sgWRN-2 (ATCCTGTGGAACATACCATG, SEQ ID NO: 6) were cultured with or without doxycycline (200 ng/ml) for three days. Doxycycline induced expression of sgWRN-1 and sgWRN-2 were designed to deplete WRN protein, and sgNT serves as a negative control. 10 million cells were collected for each condition for RNA extraction and RNAseq analysis. Experiments were performed in triplets. For RNAseq, RNA from indicated cell lines was extracted and poly-A purified. cDNA libraries from obtained RNA were sequenced using paired-ended 150 bp Illumina HiSeq platform with at least 6 Gb per sample. For each experiment, both controls and treatments were performed in triplicate. Sequencing reads were aligned to the human genome version hg38 using STAR aligner version 2.6, and the number of counts per gene were obtained using HTseq-count with gene annotations derived from Gencode release 21. Differential gene expression was analyzed using the limma-voom R-package, and gene set enrichment analysis was performed using Camera.

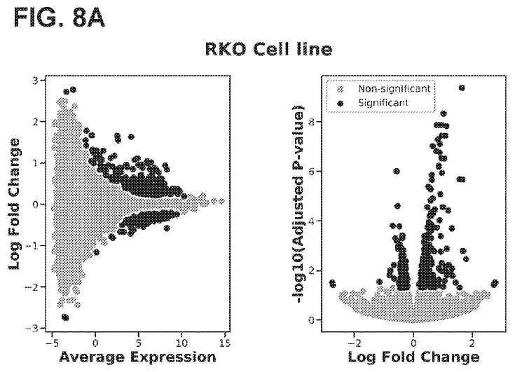

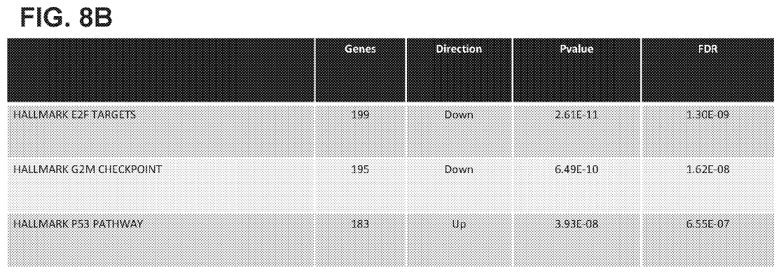

Results: As shown in FIG. 8A, a large number of genes have expression level changes upon WRN CRISPR knock-out. Among the genes that are affected, decrease of expression of E2F targets, G2M checkpoint factors, and mitotic spindle associated genes was observed (FIG. 8B). An increase in p53 is also observed. The observations are consistent with cell cycle arrest and an activation of p53 DNA damage checkpoint pathway.

### Example 9: Differential Gene Expression Analysis in HCT116 Cell Line Following WRN CRISPR Induction

The following example demonstrates that WRN knockdown by CRISPR in HCT116 cells leads to cell cycle arrest by activation of G2/M checkpoint pathway.

Procedure: HCT116-sgNT (AAGATCGAGTGCCGCATCAC, SEQ ID NO: 51), sgWRN-1 (GTAAATTGGAAAACCCACGG, SEQ ID NO: 5), or sgWRN-2 (ATCCTGTGGAACATACCATG, SEQ ID NO: 6) were cultured with or without doxycycline (200 ng/ml) for three days. Doxycycline-induced expression of sgWRN-1 and sgWRN-2 were designed to deplete WRN protein, and HCT116-sgNT served as a negative control. 10 million cells were collected for each condition for RNA extraction and RNAseq analysis. Experiments were performed in triplicate. For RNA-seq, RNA from indicated cell lines was extracted and poly-A purified. cDNA libraries from obtained RNA were sequenced using paired-ended 150 bp Illumina HiSeq platform with at least 6 Gb per sample. For each experiment, both controls and treatments were performed in triplicate. Sequencing reads were aligned to the human genome version hg38 using STAR aligner version 2.6, and the number of counts per gene were obtained using HTseq-count with gene annotations derived from Gencode release 21. Differential gene expression was analyzed using the limma-voom R-package, and gene set enrichment analysis was performed using Camera.

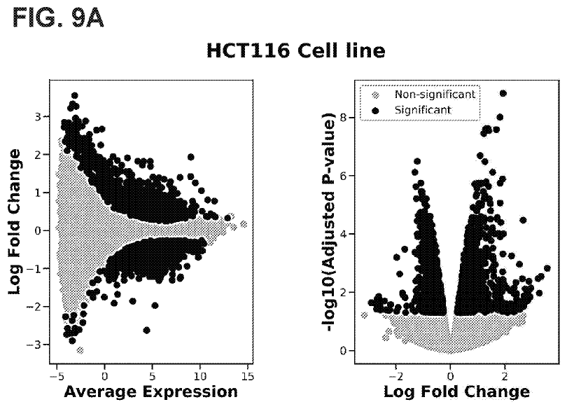

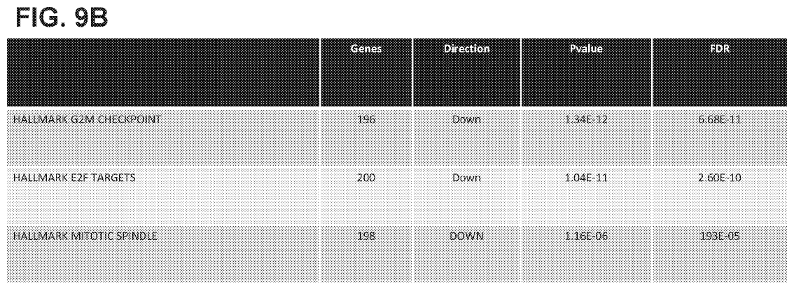

Results: As shown in FIG. 9A, a large number of genes have expression level changes upon WRN CRISPR knock-out. Among the genes that are affected, a decrease of expression of E2F targets, G2M checkpoint factors, and mitotic spindle-associated genes was observed (FIG. 9B). The observations are consistent with cell cycle arrest by activation of G2/M checkpoint pathway.

### Example 10: Differential Gene Expression Analysis in CDX Models of RKO Cell Lines Upon WRN Degradation Induction

The following example demonstrates that WRN protein depletion in a RKO (MMRd, ARID1A-mutant) xenograft in vivo model resulted in activation of p53 response pathway and DNA damage response.

Procedure: RKO and RKO-SMASh-WRN xenografts were established by subcutaneous inoculation of 10 million cells into 6-8 week old Balb/c Nude female mice. Asunaprevir (ASV) compound treatment was started when the average tumor size reach around 200 mm3. Three tumor samples from each group were collected for RNAseq analysis after 4 days of ASV treatment (7 h post last treatment). RNA from 100 mg of indicated tumors was extracted and poly-A purified. cDNA libraries from obtained RNA were sequenced using paired-ended 150 bp Illumina HiSeq platform with at least 6 Gb per sample. For each experiment both controls and treatments were performed in triplicate. Sequencing reads were first aligned to mouse genome version GRCm38 aligned using STAR aligner version 2.6. Unmapped reads were subsequently aligned to the human genome version hg38 using STAR aligner version 2.6, and the number of counts per gene were obtained using HTseq-count with gene annotations derived from Gencode release 21. Differential gene expression was analyzed using the limma-voom R-package, and gene set enrichment analysis was performed using Camera.

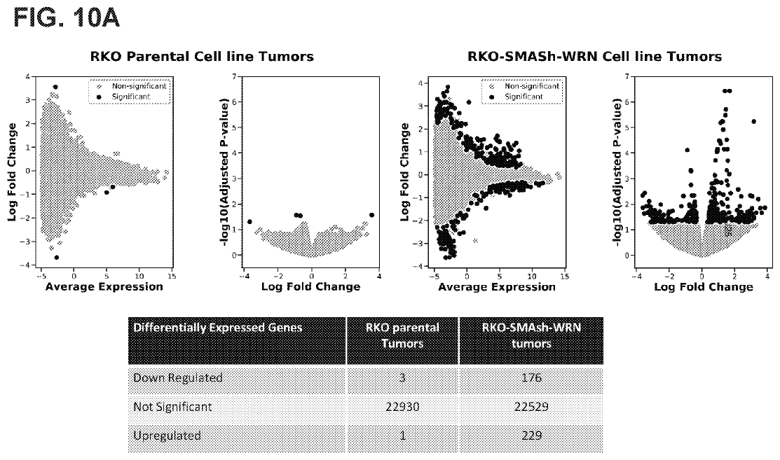

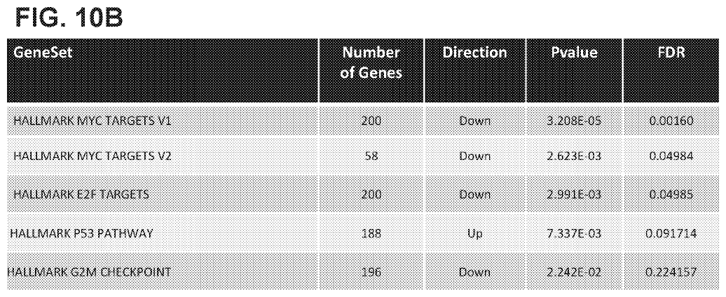

Results: Around 400 genes have expression level changes upon WRN degradation in RKO xenograft model (FIG. 10A). Gene expression is only affected by ASV treatment in animals with tumors derived from RKO parental cell line. Among the genes that were affected, an increase in expression of p53 target genes and decrease of genes that are Myc and E2F targets or are involved in G2M checkpoint was observed (FIG. 10B).

### Example 11: High Density Tiling sgRNA Screen Against WRN in RKO, HAP, SAOS2, and U2OS Cell Lines

The following example demonstrates selective dropout with WRN CRISPR tiling in the RKO cell line.

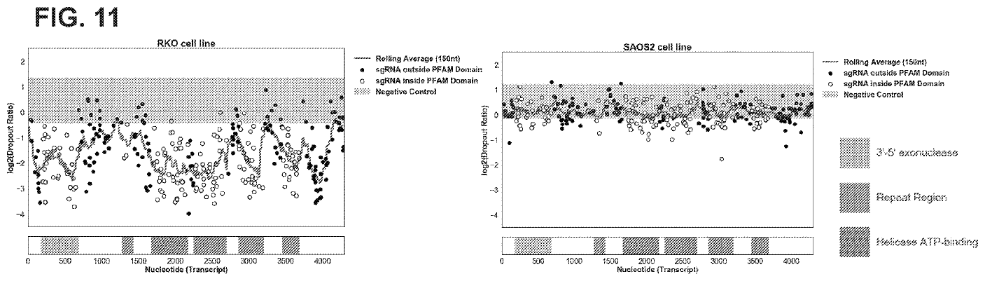

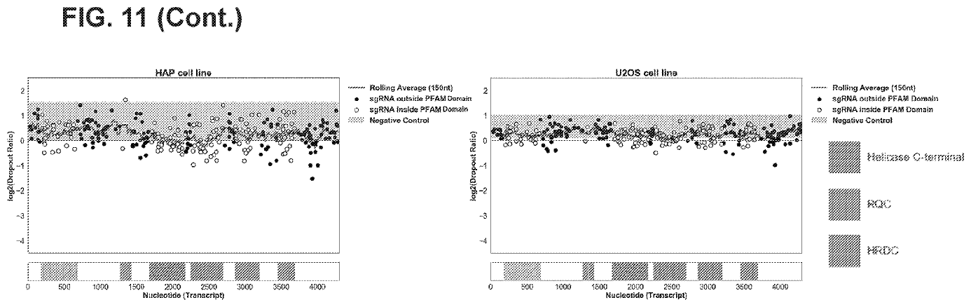

Procedure: To perform high density sgRNA tiling screens, a sgRNA library against WRN was custom synthesized at Cellecta (Mountain View, Calif.). Sequences of DNA encoding the WRN targeting sgRNAs used in this screen are listed in Table 2. Non-targeting control sequences are shown in Table 3. Procedures for virus production, cell infection, and performing the sgRNA were performed as previously described (see, e.g., Tsherniak et al, Cell 170:564-576 (2017) and Munoz et al, Cancer Discovery 6:900-913 (2016)). For each experiment, guide counts were obtained by next generation sequencing for an initial time point post infections and a final time point of the experiment. For each CRISPR guide, 50 pseudocounts were added and the log ratio between initial time point and final time point was calculated. Background was considered as the values between the 99.75 and 0.25 percentile of log ratio calculations for non-targeting guides included in the screening (grey box in FIG. 11).

Results: As shown in FIG. 11, RKO cells, but not HAP, SAOS2 or U2OS cells showed selective dropout with WRN CRISPR tiling screening. In the RKO cell line, the strongest effects were observed with guides for previously defined functional domains (e.g., helicase domain, C-terminal helix-turn-helix motif, exonuclease domain).

TABLE 2
WRN sgRNA Library

| SEQ ID | |
|---|---|
| NO | Nucleic Acid Sequence |
| 52 | TGGAAACAACTGCACAGCAG |
| 53 | AGCAGCGGAAATGTCCTGAA |
| 54 | TTCTGCACATTCATCCATTC |
| 55 | ATGTGCTGTAGAAGAAAGAA |
| 56 | CTAAACTCAAGGCATGTGTT |
| 57 | CAGTGAATTCTAAGAAGGGG |
| 58 | ATCCAGTGAATTCTAAGAAG |
| 59 | GATCCAGTGAATTCTAAGAA |
| 60 | GGATCCAGTGAATTCTAAGA |
| 61 | CTCCCCTTCTTAGAATTCAC |
| 62 | AGCATCGTAACTATACACAA |
| 63 | CTTACCTAATATCTTCTGAC |
| 64 | CTTTCCTGTCAGAAGATATT |
| 65 | TTTAGCATGAGTCTATCAGA |
| 66 | TTAGCATGAGTCTATCAGAT |
| 67 | TAGCATGAGTCTATCAGATG |
| 68 | GAGTCTATCAGATGGGGATG |
| 69 | TCTATCAGATGGGGATGTGG |
| 70 | CTATCAGATGGGGATGTGGT |
| 71 | GGATGTGGTGGGATTTGACA |
| 72 | TGGTGGGATTTGACATGGAG |
| 73 | TTCCCTCTATTGTATAATGG |
| 74 | TGGCCACCATTATACAATAG |
| 75 | GGCCACCATTATACAATAGA |
| 76 | AACCTGACATGGAAGAAACG |
| 77 | TTCCACGTTTCTTCCATGTC |
| 78 | GTAGAGATACCAACCTGACA |
| 79 | ACGTTTCTTCCATGTCAGGT |
| 80 | AATAAAGCAGTTAAAAAGGC |
| 81 | GCAGTTAAAAAGGCAGGTGT |
| 82 | AAGGCAGGTGTAGGAATTGA |
| 83 | TAGGAATTGAAGGAGATCAG |
| 84 | GACAGATGTTGCCAATAAAA |
| 85 | AACCAGACTGTTAAGGCTCC |
| 86 | GGTGTTTAACCAGACTGTTA |
| 87 | AGTCTGGTTAAACACCTCTT |
| 88 | TCAGGAGCTGTTTACCTAAG |
| 89 | AGCGGATAGACTTGTCTTTC |
| 90 | ATTTACTCCAATTGCTACAG |
| 91 | AGTCTATCCGCTGTAGCAAT |
| 92 | AGTTTCTGGTCCTCAGTGAG |
| 93 | GAGTAAATTTCCTCTCACTG |
| 94 | CAGTGGCTGCATACAGTTTC |
| 95 | AGCACGTACATAAGCATCAG |
| 96 | CATCCAAAATCTCTAAATTT |
| 97 | TTACCGAAATTTAGAGATTT |
| 98 | CAAAGGTTTGCTATAAATAA |
| 99 | TAAGTAGGATTTCTTCCTCT |
| 100 | GTTTGTTCATGTCGCTAAGT |
| 101 | ACAGTTGACTTCAATCTCTG |
| 102 | TTCAATCTCTGAGGAAGTGA |
| 103 | CTCTGAGGAAGTGATGGATC |
| 104 | TCCAATTTACTGAAAGCATG |
| 105 | TCCTCATGCTTTCAGTAAAT |
| 106 | TCAGTAAATTGGAAAACCCA |
| 107 | GTAAATTGGAAAACCCACGG |
| 108 | AGGTAATATTTAACCTCCGT |
| 109 | AAGGTAATATTTAACCTCCG |
| 110 | TAGGGTTTCTATCTTACTAA |
| 111 | CAGAAAATCTATATTCACTG |
| 112 | AAAATCTATATTCACTGAGG |
| 113 | TCACTGAGGAGGATGATAAT |
| 114 | CACTGAGGAGGATGATAATT |
| 115 | CTAACATTGAGACTGAACTG |
| 116 | AATAAGTTTAAATTATTGCT |
| 117 | TAATAAGTTTAAATTATTGC |
| 118 | CCCAGTAGTTGAATCTTCAA |
| 119 | TCCTTTGAAGATTCAACTAC |
| 120 | CCTTTGAAGATTCAACTACT |
| 121 | CTTTGAAGATTCAACTACTG |
| 122 | TTTGAAGATTCAACTACTGG |
| 123 | TTCACGTTGAAGATGAAACA |
| 124 | TCACGTTGAAGATGAAACAT |
| 125 | TAGCTAAATGATCAAGTGTT |
| 126 | TTAGCTAAATGATCAAGTGT |
| 127 | GATCATTTAGCTAAACATGA |
| 128 | CATGATGGAGAAGATGTACT |
| 129 | AGATGTACTTGGAAATAAAG |
| 130 | CAACAAATTGAAAGAGAATA |
| 131 | GAGACTGCTGTTCCAAAATT |
| 132 | TATTGCTTATAAATCTACTG |
| 133 | TTCATCACTCTCAATTACAT |
| 134 | GAGTGATGAAGATTTAGAAA |
| 135 | TTTAGAAATGGAGATGCTTA |
| 136 | GTTCTACCGTGCCACTATTG |
| 137 | TCTTTAGAAAACCTCAATAG |
| 138 | AGAAAACCTCAATAGTGGCA |
| 139 | TTTAAGCATTTAGAATGAGT |
| 140 | TCATTCTAAATGCTTAAAAA |
| 141 | CTTAAAAATGGAAAGAAATC |
| 142 | TTAAAAATGGAAAGAAATCT |
| 143 | GATGAAAATGAAGCTAATGA |
| 144 | ATGAAAATGAAGCTAATGAA |
| 145 | TGAAAATGAAGCTAATGAAG |
| 146 | GGTGCTGGCCACAAAAAGTC |
| 147 | ACTTGCTCTTCATTGGGTGC |
| 148 | CAAGTAACTTGCTCTTCATT |
| 149 | GCAAGTAACTTGCTCTTCAT |
| 150 | AATGGCCAAAGTACATCTTG |
| 151 | ACTTGCCTCAAGATGTACTT |
| 152 | CTCACGGTTTAAAACTGGAA |
| 153 | TTATACTCACGGTTTAAAAC |
| 154 | TTTCACCTTCAAGAGTTCAG |
| 155 | AAGAGATAATGTTGCTGTCA |
| 156 | AATGTTGCTGTCATGGCAAC |
| 157 | CTACATAAACAGGTGGATAC |
| 158 | CCAATCTTGCCTACATAAAC |
| 159 | CAGTATCCACCTGTTTATGT |
| 160 | AAATAAGGGGAGAGATAACA |
| 161 | GTCTTCCATCAGAGAAATAA |
| 162 | GGTCTTCCATCAGAGAAATA |
| 163 | CTCTCCCCTTATTTCTCTGA |
| 164 | CTTACTTAAGCTGTAGCACT |
| 165 | AAGGAAGCAAGCTGGGATGT |
| 166 | CTGATCCAAGGAAGCAAGCT |
| 167 | GCTGATCCAAGGAAGCAAGC |
| 168 | AACATCCCAGCTTGCTTCCT |
| 169 | TTTCTGACTGTGCTGATCCA |
| 170 | CTGGAGTTACGTATACAATC |
| 171 | GTAACTCCAGAATACTGTTC |
| 172 | AGAATACTGTTCAGGTAACA |
| 173 | CAGCCTCAAGTTGCTGGAGC |
| 174 | CAATATCAGCCTCAAGTTGC |
| 175 | GGGCCTGCTCCAGCAACTTG |
| 176 | CAGCAACTTGAGGCTGATAT |
| 177 | AGGTATCACGCTCATTGCTG |
| 178 | CACGCTCATTGCTGTGGATG |
| 179 | AGGCTCACTGTATTTCTGAG |
| 180 | GGCTCACTGTATTTCTGAGT |
| 181 | GCTCACTGTATTTCTGAGTG |
| 182 | CTCACTGTATTTCTGAGTGG |
| 183 | TAGGGATTCATTCAGGAAGT |
| 184 | AGGGATTCATTCAGGAAGTT |
| 185 | CATTGGCAGTGCTGTCTTTA |
| 186 | CCATTGGCAGTGCTGTCTTT |
| 187 | CCTAAAGACAGCACTGCCAA |
| 188 | GACTTGGCAAAGCTTACCAT |
| 189 | GTAGCAGTAAGTGCAACGAT |
| 190 | CTACTGCAAGTTCTTCAATC |
| 191 | TACTGCAAGTTCTTCAATCC |
| 192 | AAACCAGTACAGGTGATCTG |
| 193 | AATCCTCAGATCACCTGTAC |
| 194 | TGGTCGATCAAAACCAGTAC |
| 195 | CTAACTTCTAAATACAGGTT |
| 196 | TTCGCCTAACTTCTAAATAC |
| 197 | CAAACCTGTATTTAGAAGTT |
| 198 | TTAGAAGTTAGGCGAAAAAC |
| 199 | TAGAAGTTAGGCGAAAAACA |
| 200 | AAAAACAGGGAATATCCTTC |
| 201 | GAAATGGCTGCAGATCCTGA |
| 202 | AGCCATTTCTTGTCAAAACA |
| 203 | TGGACCTTCAAATTCCCAGT |
| 204 | TTGGACCTTCAAATTCCCAG |
| 205 | AGTTCCCACTGGGAATTTGA |
| 206 | GAAGGACAGTAGATGATTGT |
| 207 | AAAATGACACAACAAGTTAC |
| 208 | AACAAGTTACAGGTGAACTT |
| 209 | AGGAAACTGAATCTATCCTG |
| 210 | GCCCGCATGGTATGTTCCAC |
| 211 | ATCCTGTGGAACATACCATG |
| 212 | TCCTGTGGAACATACCATGC |
| 213 | TGCTAAAACTCATGCCCGCA |
| 214 | CAAGGAAAGACATTCATCAT |
| 215 | GTTTGTAAGAGATGAAATTC |
| 216 | AATGCCCATTCCAAAAGCTA |
| 217 | GTCATAGCTACCATAGCTTT |
| 218 | GAGCACCGTAATGAATGACT |
| 219 | ATTCGCCAAGTCATTCATTA |
| 220 | CATTCATTACGGTGCTCCTA |
| 221 | TAATATGATTCCATGTCCTT |
| 222 | TTACGGTGCTCCTAAGGACA |
| 223 | GGACATGGAATCATATTATC |
| 224 | GAATCATATTATCAGGAGAT |
| 225 | TATCAGGAGATTGGTAGAGC |
| 226 | ATTGGTAGAGCTGGTCGTGA |
| 227 | AAAGTTCTTGTCACGTCCTC |
| 228 | AAGTTCTTGTCACGTCCTCT |
| 229 | TAATGTCTGCAGGAGCCCAG |
| 230 | CTATTTAAGTTAATGTCTGC |
| 231 | CTGCAGACATTAACTTAAAT |
| 232 | CATTACGTATCTCAGTAAGA |
| 233 | ATTATACAAATTAAAGATGA |
| 234 | ATTAAAGATGATGGCAAAGA |
| 235 | ATCTTCATTCTAGCAGATGT |
| 236 | ACAAGTACAAAAAGCCTCCT |
| 237 | CAAGTACAAAAAGCCTCCTT |
| 238 | AGTTCCCATAATTCCCAAGG |
| 239 | TTCAGTTCCCATAATTCCCA |
| 240 | AAAAGCCTCCTTGGGAATTA |
| 241 | AAAGCCTCCTTGGGAATTAT |
| 242 | AAAAATGCTGTGATAATTGC |
| 243 | GCTGTGATAATTGCAGGTCC |
| 244 | ATTGGATCATTGCTATTCCA |
| 245 | TGTATCCTCTGAGTCATCCA |
| 246 | CTATTCCATGGATGACTCAG |
| 247 | ATGACTCAGAGGATACATCC |
| 248 | TGACTCAGAGGATACATCCT |
| 249 | TGCTTGTGGACCAAAGTCCC |
| 250 | GAGGATACATCCTGGGACTT |
| 251 | GACAAAAGCTTAAATGCTTG |
| 252 | TTGTCTGCTGTGGACATCTT |
| 253 | GACATCTTAGGCGAAAAATT |
| 254 | TTAGGCGAAAAATTTGGAAT |
| 255 | TAGGCGAAAAATTTGGAATT |
| 256 | GATCCTCGGAGAAATAAAAT |
| 257 | GATATATACTTACAGATCCT |
| 258 | ACTGTGCCTGCGATATTGAT |
| 259 | GTCTTGCCGATCAATATCGC |
| 260 | TATCGCAGGCACAGTTTATT |
| 261 | AGGCACAGTTTATTTGGCAC |
| 262 | CAGTTTATTTGGCACTGGCA |
| 263 | GCAAGGATCAAACAGAGAGT |
| 264 | AGGATCAAACAGAGAGTTGG |
| 265 | TCAAACAGAGAGTTGGTGGA |
| 266 | TCCCTCAGTGATCAGCTGAC |
| 267 | ATCCCTCAGTGATCAGCTGA |
| 268 | TTCCCGTCAGCTGATCACTG |
| 269 | TCCCGTCAGCTGATCACTGA |
| 270 | GCTGATCACTGAGGGATTCT |
| 271 | GATTCTTGGTAGAAGTTTCT |
| 272 | GATTTGCGCCCTTACGAAAA |
| 273 | CTTCATTAGCTTGAAGGATG |
| 274 | ACAATTCTTCATTAGCTTGA |
| 275 | CTAGGCAGAAGCAACTTCTT |
| 276 | CAAAGAAGTTGCTTCTGCCT |
| 277 | TAGTTCGAAAACTGTATCTT |
| 278 | AGTTCGAAAACTGTATCTTC |
| 279 | TTGATTATAACAATGCTCTT |
| 280 | TTCTCTGTACTTAATTCAAC |
| 281 | TGAATTAAGTACAGAGAAGA |
| 282 | TGTTTAAATGCAGTCTAACT |
| 283 | CCAGAAGAAATCTTATCACA |
| 284 | CCATGTGATAAGATTTCTTC |
| 285 | CATGTGATAAGATTTCTTCT |
| 286 | GGAGTAACATTTCTAAAAAA |
| 287 | TGCCGAAATAACAGGCTGTG |
| 288 | TGCTCTTGTGCCGAAATAAC |
| 289 | TTCCTCACAGCCTGTTATTT |
| 290 | TGTTATTTCGGCACAAGAGC |
| 291 | GGCACAAGAGCAGGAGACTC |
| 292 | TTTGTTCAGATTGTGTTATA |
| 293 | GATTGTGTTATATGGCAAAT |
| 294 | ATGGCAAATTGGTAGAAGCT |
| 295 | GCAGAAACATGCCAATAAAA |
| 296 | TTTGTTGCCAGAATAGCTGG |
| 297 | GTTTGTTGCCAGAATAGCTG |
| 298 | TGTTTGTTGCCAGAATAGCT |
| 299 | TTGTTTGTTGCCAGAATAGC |
| 300 | GGATGTTCCCCCAGCTATTC |
| 301 | TCTGGCAACAAACAAGATAC |
| 302 | GGCAACAAACAAGATACTGG |
| 303 | AAACAAGATACTGGTGGATA |
| 304 | AAAGATAGTTTACCTCATTT |
| 305 | TGTTAAACACAGACCAACTA |
| 306 | CTACGGTTGAAAACGTAAAA |
| 307 | GAAAACGTAAAAAGGATTGA |
| 308 | AGGATTGATGGTGTTTCTGA |
| 309 | TGAAGGCAAAGCTGCCATGT |
| 310 | TTCCAACAGAGGGGCCAACA |
| 311 | TTTGATGACTTCCAACAGAG |
| 312 | GTTTGATGACTTCCAACAGA |
| 313 | TGTTTGATGACTTCCAACAG |
| 314 | TTACCTGAACACTATTTGTT |
| 315 | CTGCCAAACAAATAGTGTTC |
| 316 | AGAAGAACAGAAGACGAGTC |
| 317 | ATGCACACTTTCACAGTCTA |
| 318 | TTGGAATAAAGAGTATGTGA |
| 319 | CCAAGAAAAGAAGATGCCTT |
| 320 | ATGAAAGTCACACTTACCAA |
| 321 | AGAAGAGCATAGCTGAGAGC |
| 322 | TGCATGCCAATTGTCATGAG |
| 323 | ATTCTGCCTCTCATGACAAT |
| 324 | TGGCATGCACTTATCCCAAG |
| 325 | GCAGCCAGCTTTCACCGCTT |
| 326 | GGCAGCCAGCTTTCACCGCT |
| 327 | TTATCCCAAGCGGTGAAAGC |
| 328 | CTGCTCGCTCCAAATCAAGG |
| 329 | CCTGCTCGCTCCAAATCAAG |
| 330 | GCCTGCTCGCTCCAAATCAA |
| 331 | GGCCTGCTCGCTCCAAATCA |
| 332 | CCCCTTGATTTGGAGCGAGC |
| 333 | GCGAGCAGGCCTGACTCCAG |
| 334 | GCAATAATCTTCTGAACCTC |
| 335 | CTGAGTTGACGGGAGGGTTT |
| 336 | TCTCACCTGAGTTGACGGGA |
| 337 | CTCTCACCTGAGTTGACGGG |
| 338 | ATGCCTCTCACCTGAGTTGA |
| 339 | CGAAACCCTCCCGTCAACTC |
| 340 | CAGGAACTAACATTCTGATT |
| 341 | TCTCAATTGCCATGTGGATA |
| 342 | TGACACGTACCTTATCCACA |
| 343 | TAAGGATCTCAATTGCCATG |
| 344 | CGCTGTCAGGACCATGTTTA |
| 345 | GCAATTGAGATCCTTAAACA |
| 346 | GAAGGTTGAAGTCCGCTGTC |
| 347 | CTTCATGTGATGTCAACAAA |
| 348 | GAACAGATCTCTTCAGAACC |
| 349 | TGAACAGATCTCTTCAGAAC |
| 350 | TTCAAGTTCTAAGAGAAGCA |
| 351 | AGAAGTAGGCATCAATACTG |
| 352 | GAAAGAGACGATTACCTGTG |
| 353 | CTTCCTTTGGCAAACCACAC |
| 354 | TTACCTGTGTGGTTTGCCAA |
| 355 | CTTGCTGGTATCACTTCCTT |
| 356 | TTTGTCCATTAATTTCTTGC |
| 357 | TGATACCAGCAAGAAATTAA |
| 358 | AATTAATGGACAAAACGAAA |
| 359 | ATTAATGGACAAAACGAAAA |
| 360 | TTAATGGACAAAACGAAAAG |
| 361 | ATGGACAAAACGAAAAGGGG |

TABLE 3
Non-targeting control sgRNAs

| SEQ ID NO | Nucleic Acid Sequence |
|---|---|
| 362 | GTAGCGAACGTGTCCGGCGT |
| 363 | GACCGGAACGATCTCGCGTA |
| 364 | GGCAGTCGTTCGGTTGATAT |
| 365 | GCTTGAGCACATACGCGAAT |
| 366 | GTGGTAGAATAACGTATTAC |
| 367 | GTCATACATGGATAAGGCTA |
| 368 | GATACACGAAGCATCACTAG |
| 369 | GAACGTTGGCACTACTTCAC |
| 370 | GATCCATGTAATGCGTTCGA |
| 371 | GTCGTGAAGTGCATTCGATC |
| 372 | GTTCGACTCGCGTGACCGTA |
| 373 | GAATCTACCGCAGCGGTTCG |
| 374 | GAAGTGACGTCGATTCGATA |
| 375 | GCGGTGTATGACAACCGCCG |
| 376 | GTACCGCGCCTGAAGTTCGC |
| 377 | GCAGCTCGTGTGTCGTACTC |
| 378 | GCGCCTTAAGAGTACTCATC |
| 379 | GAGTGTCGTCGTTGCTCCTA |
| 380 | GCAGCTCGACCTCAAGCCGT |
| 381 | GTATCCTGACCTACGCGCTG |
| 382 | GTGTATCTCAGCACGCTAAC |
| 383 | GTCGTCATACAACGGCAACG |
| 384 | GTCGTGCGCTTCCGGCGGTA |
| 385 | GCGGTCCTCAGTAAGCGCGT |
| 386 | GCTCTGCTGCGGAAGGATTC |
| 387 | GCATGGAGGAGCGTCGCAGA |
| 388 | GTAGCGCGCGTAGGAGTGGC |
| 389 | GATCACCTGCATTCGTACAC |
| 390 | GCACACCTAGATATCGAATG |
| 391 | GTTGATCAACGCGCTTCGCG |
| 392 | GCGTCTCACTCACTCCATCG |
| 393 | GCCGACCAACGTCAGCGGTA |
| 394 | GGATACGGTGCGTCAATCTA |
| 395 | GAATCCAGTGGCGGCGACAA |
| 396 | GCACTGTCAGTGCAACGATA |
| 397 | GCGATCCTCAAGTATGCTCA |
| 398 | GCTAATATCGACACGGCCGC |
| 399 | GGAGATGCATCGAAGTCGAT |
| 400 | GGATGCACTCCATCTCGTCT |
| 401 | GTGCCGAGTAATAACGCGAG |
| 402 | GAGATTCCGATGTAACGTAC |
| 403 | GTCGTCACGAGCAGGATTGC |
| 404 | GCGTTAGTCACTTAGCTCGA |
| 405 | GTTCACACGGTGTCGGATAG |
| 406 | GGATAGGTGACCTTAGTACG |
| 407 | GTATGAGTCAAGCTAATGCG |
| 408 | GCAACTATTGGAATACGTGA |
| 409 | GTTACCTTCGCTCGTCTATA |
| 410 | GTACCGAGCACCACAGGCCG |
| 411 | GTCAGCCATCGGATAGAGAT |
| 412 | GTACGGCACTCCTAGCCGCT |
| 413 | GGTCCTGTCGTATGCTTGCA |
| 414 | GCCGCAATATATGCGGTAAG |
| 415 | GCGCACGTATAATCCTGCGT |
| 416 | GTGCACAACACGATCCACGA |
| 417 | GCACAATGTTGACGTAAGTG |
| 418 | GTAAGATGCTGCTCACCGTG |
| 419 | GTCGGTGATCCAACGTATCG |
| 420 | GAGCTAGTAGGACGCAAGAC |
| 421 | GTACGTGGAAGCTTGTGGCC |
| 422 | GAGAACTGCCAGTTCTCGAT |
| 423 | GCCATTCGGCGCGGCACTTC |
| 424 | GCACACGACCAATCCGCTTC |
| 425 | GAGGTGATCGATTAAGTACA |
| 426 | GTCACTCGCAGACGCCTAAC |
| 427 | GCGCTACGGAATCATACGTT |
| 428 | GGTAGGACCTCACGGCGCGC |
| 429 | GAACTGCATCTTGTTGTAGT |
| 430 | GATCCTGATCCGGCGGCGCG |
| 431 | GGTATGCGCGATCCTGAGTT |
| 432 | GCGGAGCTAGAGAGCGGTCA |
| 433 | GAATGGCAATTACGGCTGAT |
| 434 | GTATGGTGAGTAGTCGCTTG |
| 435 | GTGTAATTGCGTCTAGTCGG |
| 436 | GGTCCTGGCGAGGAGCCTTG |
| 437 | GAAGATAAGTCGCTGTCTCG |
| 438 | GTCGGCGTTCTGTTGTGACT |
| 439 | GAGGCAAGCCGTTAGGTGTA |
| 440 | GCGGATCCAGATCTCATTCG |
| 441 | GGAACATAGGAGCACGTAGT |
| 442 | GTCATCATTATGGCGTAAGG |
| 443 | GCGACTAGCGCCATGAGCGG |
| 444 | GGCGAAGTTCGACATGACAC |
| 445 | GCTGTCGTGTGGAGGCTATG |
| 446 | GCGGAGAGCATTGACCTCAT |
| 447 | GACTAATGGACCAAGTCAGT |
| 448 | GCGGATTAGAGGTAATGCGG |
| 449 | GCCGACGGCAATCAGTACGC |
| 450 | GTAACCTCTCGAGCGATAGA |
| 451 | GACTTGTATGTGGCTTACGG |
| 452 | GTCACTGTGGTCGAACATGT |
| 453 | GTACTCCAATCCGCGATGAC |
| 454 | GCGTTGGCACGATGTTACGG |
| 455 | GAACCAGCCGGCTAGTATGA |
| 456 | GTATACTAGCTAACCACACG |
| 457 | GAATCGGAATAGTTGATTCG |
| 458 | GAGCACTTGCATGAGGCGGT |
| 459 | GAACGGCGATGAAGCCAGCC |
| 460 | GCAACCGAGATGAGAGGTTC |
| 461 | GCAAGATCAATATGCGTGAT |
| 462 | ACGGAGGCTAAGCGTCGCAA |
| 463 | CGCTTCCGCGGCCCGTTCAA |
| 464 | ATCGTTTCCGCTTAACGGCG |
| 465 | GTAGGCGCGCCGCTCTCTAC |
| 466 | CCATATCGGGGCGAGACATG |
| 467 | TACTAACGCCGCTCCTACAG |
| 468 | TGAGGATCATGTCGAGCGCC |
| 469 | GGGCCCGCATAGGATATCGC |
| 470 | TAGACAACCGCGGAGAATGC |
| 471 | ACGGGCGGCTATCGCTGACT |
| 472 | CGCGGAAATTTTACCGACGA |
| 473 | CTTACAATCGTCGGTCCAAT |
| 474 | GCGTGCGTCCCGGGTTACCC |
| 475 | CGGAGTAACAAGCGGACGGA |
| 476 | CGAGTGTTATACGCACCGTT |
| 477 | CGACTAACCGGAAACTTTTT |
| 478 | CAACGGGTTCTCCCGGCTAC |
| 479 | CAGGAGTCGCCGATACGCGT |
| 480 | TTCACGTCGTCTCGCGACCA |
| 481 | GTGTCGGATTCCGCCGCTTA |
| 482 | CACGAACTCACACCGCGCGA |
| 483 | CGCTAGTACGCTCCTCTATA |
| 484 | TCGCGCTTGGGTTATACGCT |
| 485 | CTATCTCGAGTGGTAATGCG |
| 486 | AATCGACTCGAACTTCGTGT |
| 487 | CCCGATGGACTATACCGAAC |
| 488 | ACGTTCGAGTACGACCAGCT |
| 489 | CGCGACGACTCAACCTAGTC |
| 490 | GGTCACCGATCGAGAGCTAG |
| 491 | CTCAACCGACCGTATGGTCA |
| 492 | CGTATTCGACTCTCAACGCG |
| 493 | CTAGCCGCCCAGATCGAGCC |
| 494 | GAATCGACCGACACTAATGT |
| 495 | ACTTCAGTTCGGCGTAGTCA |
| 496 | GTGCGATGTCGCTTCAACGT |
| 497 | CGCCTAATTTCCGGATCAAT |
| 498 | CGTGGCCGGAACCGTCATAG |
| 499 | ACCCTCCGAATCGTAACGGA |
| 500 | AAACGGTACGACAGCGTGTG |
| 501 | ACATAGTCGACGGCTCGATT |
| 502 | GATGGCGCTTCAGTCGTCGG |
| 503 | ATAATCCGGAAACGCTCGAC |
| 504 | CGCCGGGCTGACAATTAACG |
| 505 | CGTCGCCATATGCCGGTGGC |
| 506 | CGGGCCTATAACACCATCGA |
| 507 | CGCCGTTCCGAGATACTTGA |
| 508 | CGGGACGTCGCGAAAATGTA |
| 509 | TCGGCATACGGGACACACGC |
| 510 | AGCTCCATCGCCGCGATAAT |
| 511 | ATCGTATCATCAGCTAGCGC |
| 512 | TCGATCGAGGTTGCATTCGG |
| 513 | CTCGACAGTTCGTCCCGAGC |
| 514 | CGGTAGTATTAATCGCTGAC |
| 515 | TGAACGCGTGTTTCCTTGCA |
| 516 | CGACGCTAGGTAACGTAGAG |
| 517 | CATTGTTGAGCGGGCGCGCT |
| 518 | CCGCTATTGAAACCGCCCAC |
| 519 | AGACACGTCACCGGTCAAAA |
| 520 | TTTACGATCTAGCGGCGTAG |
| 521 | TTCGCACGATTGCACCTTGG |
| 522 | GGTTAGAGACTAGGCGCGCG |
| 523 | CCTCCGTGCTAACGCGGACG |
| 524 | TTATCGCGTAGTGCTGACGT |
| 525 | TACGCTTGCGTTTAGCGTCC |
| 526 | CGCGGCCCACGCGTCATCGC |
| 527 | AGCTCGCCATGTCGGTTCTC |
| 528 | AACTAGCCCGAGCAGCTTCG |
| 529 | CGCAAGGTGTCGGTAACCCT |
| 530 | CTTCGACGCCATCGTGCTCA |
| 531 | TCCTGGATACCGCGTGGTTA |
| 532 | ATAGCCGCCGCTCATTACTT |
| 533 | GTCGTCCGGGATTACAAAAT |
| 534 | TAATGCTGCACACGCCGAAT |
| 535 | TATCGCTTCCGATTAGTCCG |
| 536 | GTACCATACCGCGTACCCTT |
| 537 | TAAGATCCGCGGGTGGCAAC |
| 538 | GTAGACGTCGTGAGCTTCAC |
| 539 | TCGCGGACATAGGGCTCTAA |
| 540 | AGCGCAGATAGCGCGTATCA |
| 541 | GTTCGCTTCGTAACGAGGAA |
| 542 | GACCCCCGATAACTTTTGAC |
| 543 | ACGTCCATACTGTCGGCTAC |
| 544 | GTACCATTGCCGGCTCCCTA |
| 545 | TGGTTCCGTAGGTCGGTATA |
| 546 | TCTGGCTTGACACGACCGTT |
| 547 | CGCTAGGTCCGGTAAGTGCG |
| 548 | AGCACGTAATGTCCGTGGAT |
| 549 | AAGGCGCGCGAATGTGGCAG |
| 550 | ACTGCGGAGCGCCCAATATC |
| 551 | CGTCGAGTGCTCGAACTCCA |
| 552 | TCGCAGCGGCGTGGGATCGG |
| 553 | ATCTGTCCTAATTCGGATCG |
| 554 | TGCGGCGTAATGCTTGAAAG |
| 555 | CGAACTTAATCCCGTGGCAA |
| 556 | GCCGTGTTGCTGGATACGCC |
| 557 | TACCCTCCGGATACGGACTG |
| 558 | CCGTTGGACTATGGCGGGTC |
| 559 | GTACGGGGCGATCATCCACA |
| 560 | AAGAGTAGTAGACGCCCGGG |
| 561 | AAGAGCGAATCGATTTCGTG |

### Other Embodiments

All publications, patents, and patent applications mentioned in this specification are incorporated herein by reference in their entirety to the same extent as if each individual publication, patent, or patent application was specifically and individually indicated to be incorporated by reference in its entirety.

Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claimed.

1. A method of treating cancer having a mutation that results in a loss of function of AT-Rich Interaction Domain 1A (ARID1A) in a subject in need thereof, the method comprising administering to the subject an effective amount of an agent that reduces the level and/or activity of Werner Syndrome RecQ Like Helicase (WRN) in a cell in the subject.

2. A method of reducing the level and/or activity of WRN in a cancer cell having a mutation that results in a loss of function of ARID1A in a subject, the method comprising contacting the cell with an effective amount of an agent that reduces the level and/or activity of WRN in the cell.

3. A method of reducing tumor growth of a cancer having a mutation that results in a loss of function of ARID1A in a subject, the method comprising administering to the subject an effective amount of an agent that reduces the level and/or activity of WRN in a cell in the subject.

4. A method of treating cancer having a mismatch repair deficiency (MMRd) in a subject in need thereof, the method comprising administering to the subject an effective amount of an agent that reduces the level and/or activity of WRN in a cell in the subject.

5. A method of reducing the level and/or activity of WRN in a cancer cell having a MMRd in a subject, the method comprising contacting the cell with an effective amount of an agent that reduces the level and/or activity of WRN in the cell.

6. A method of reducing tumor growth of a cancer having a MMRd in a subject in need thereof, the method comprising administering to the subject an effective amount of an agent that reduces the level and/or activity of WRN in a cell in the subject.

7. A method of treating cancer having a microsatellite instability (MSI)-positive phenotype in a subject in need thereof, the method comprising administering to the subject an effective amount of an agent that reduces the level and/or activity of WRN in a cell in the subject.

8. A method of reducing the level and/or activity of WRN in cancer cell having an MSI-positive phenotype in a subject, the method comprising contacting the cell with an effective amount of an agent that reduces the level and/or activity of WRN in the cell.

9. A method of reducing tumor growth of a cancer having an MSI-positive phenotype in a subject in need thereof, the method comprising administering to the subject an effective amount of an agent that reduces the level and/or activity of WRN in a cell in the subject.

10. The method of any one of claims 1-9, wherein the activity of WRN is WRN helicase activity.

11. The method of any one of claims 4-10, wherein the MMRd is associated with a mutation in the MLH1, MLH3, MSH2, MSH3, MSH6, PMS1, PMS2, and/or EPCAM genes.

12. The method of claim 11, wherein the MMRd is associated with a mutation in the MLH1, MSH2, MSH6, PMS2, and/or EPCAM genes.

13. The method of claim 12, wherein the MMRd is associated with a mutation in the MLH1 gene.

14. The method of any one of claims 4-13, wherein the cancer has an MSI-positive phenotype characterized by the presence of an MSI at least one of the mononucleotide or dinucleotide markers selected from the group consisting of BAT25, BAT26, D2S123, D5S346, and D17S250.

15. The method of claim 14, wherein the MSI-positive phenotype is an MSI-high (MSI-H) phenotype characterized by the presence of an MSI at least two of the mononucleotide or dinucleotide markers selected from the group consisting of BAT25, BAT26, D2S123, D5S346, and D17S250.

16. The method of any one of claims 4-15, wherein the cancer has a mutation that results in a loss of function of ARID1A.

17. The method of any one of claims 1-16, wherein the agent that reduces the level and/or activity of WRN in a cell in the subject is directed to or targets one or more domains of WRN selected from the group consisting of a helicase domain, an endonuclease domain, a RecQ C-terminal domain, and/or a C-terminal helix-turn-helix motif.

18. The method of any one of claims 1-17, wherein the agent that reduces the level and/or activity of WRN is a nuclease, a polynucleotide, a small molecule compound, an antibody, and/or an enzyme.

19. The method of claim 18, wherein the agent that reduces the level and/or activity of WRN is a nuclease.

20. The method of claim 19, wherein the nuclease is a clustered regularly interspaced short palindromic repeats (CRISPR)-associated protein.

21. The method of claim 20, wherein the CRISPR-associated protein is CRISPR-associated protein 9 (Cas9).

22. The method of claim 19, wherein the nuclease is a transcription activator-like effector nuclease (TALEN), a meganuclease, or a zinc finger nuclease (ZFN).

23. The method of claim 18, wherein the agent that reduces the level and/or activity of WRN is a polynucleotide.

24. The method of claim 23, wherein the polynucleotide is an antisense nucleic acid, a CRISPR/Cas 9 nucleotide, a short interfering RNA (siRNA), a short hairpin RNA (shRNA), a micro RNA (miRNA), or a ribozyme.

25. The method of claim 24, wherein the polynucleotide comprises a sequence having at least 85% sequence identity to the nucleic acid sequence of any one of SEQ ID NOs: 5-50.

26. The method of claim 25, wherein the polynucleotide comprises a sequence having at least 85% sequence identity to the nucleic acid sequence of any one of SEQ ID NOs: 5-10.

27. The method of any one of claims 1-26, wherein the method further comprises administering to the subject an anti-cancer therapy.

28. The method of claim 27, wherein the anti-cancer therapy is an immunotherapy.

29. The method of claim 28, wherein the immunotherapy is a CTLA-4 inhibitor, a PD-1 inhibitor, a PD-L1 inhibitor, or adoptive T cell transfer therapy.

30. The method of claim 27, wherein the anti-cancer therapy is a chemotherapy.

31. The method of any one of claims 1-30, wherein the agent that reduces the level and/or of WRN is administered systemically or intratumorally to the subject.

32. The method of any one of claims 1-31, wherein the cancer is an MSI-positive cancer, an MSI-H cancer, an adrenocortical carcinoma, a bladder carcinoma, a breast carcinoma, a cervical squamous cell carcinoma, an endocervical adenocarcinoma, a cholangiocarcinoma, a chronic lymphocytic leukemia, a colorectal cancer, a colon adenocarcinoma, a cutaneous T-cell lymphoma, a lymphoid neoplasm diffuse large B-cell lymphoma, an esophageal carcinoma, a glioblastoma multiforme, a head and neck squamous cell carcinoma, a kidney chromophobe, a kidney renal papillary cell carcinoma, an acute myeloid leukemia, a lower-grade glioma, a liver hepatocellular carcinoma, a lung adenocarcinoma, a lung squamous cell carcinoma, a mesothelioma, a nasopharyngeal carcinoma, an ovarian cancer, an ovarian serous cystadenocarcinoma, a pancreatic adenocarcinoma, a pheochromocytoma, paraganglioma, a prostate adenocarcinoma, a rectal adenocarcinoma, a sarcoma, a skin cutaneous melanoma, a stomach adenocarcinoma, a testicular germ cell tumor, a thyroid carcinoma, a thymoma, an uterine corpus endometrial carcinoma, an uterine carcinosarcoma, an uveal melanoma, a pediatric acute myeloid leukemia, a pediatric neuroblastoma, or a pediatric high-risk Wilms tumor.

33. The method of claim 32, wherein the cancer is an MSI-positive cancer, an MSI-H cancer, an ovarian cancer, a uterine corpus endometrial carcinoma, a colorectal cancer, a colon adenocarcinoma, or a stomach adenocarcinoma.

34. The method of any one of claims 1-33, wherein the subject is a human.


SEQUENCE LISTING (561 sequences)

SEQ ID NO: 1 - protein, 1432 aa, Homo sapiens
MSEKKLETTAQQRKCPEWMNVQNKRCAVEERKACVRKSVFEDDLPFLEFTGSIVYSYDASDCSFLSEDISMSLSDGDVVGFDMEWPPLYNRGKLGKVALIQLCVSESKCYLFHVSSMSVFPQGLKMLLENKAVKKAGVGIEGDQWKLLRDFDIKLKNFVELTDVANKKLKCTETWSLNSLVKHLLGKQLLKDKSIRCSNWSKFPLTEDQKLYAATDAYAGFIIYRNLEILDDTVQRFAINKEEEILLSDMNKQLTSISEEVMDLAKHLPHAFSKLENPRRVSILLKDISENLYSLRRMIIGSTNIETELRPSNNLNLLSFEDSTTGGVQQKQIREHEVLIHVEDETWDPTLDHLAKHDGEDVLGNKVERKEDGFEDGVEDNKLKENMERACLMSLDITEHELQILEQQSQEEYLSDIAYKSTEHLSPNDNENDTSYVIESDEDLEMEMLKHLSPNDNENDTSYVIESDEDLEMEMLKSLENLNSGTVEPTHSKCLKMERNLGLPTKEEEEDDENEANEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKLLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSTKPQEEQKTSLVAKNKICTLSQSMAITYSLFQEKKMPLKSIAESRILPLMTIGMHLSQAVKAGCPLDLERAGLTPEVQKIIADVIRNPPVNSDMSKISLIRMLVPENIDTYLIHMAIEILKHGPDSGLQPSCDVNKRRCFPGSEEICSSSKRSKEEVGINTETSSAERKRRLPVWFAKGSDTSKKLMDKTKRGGLFS

SEQ ID NO: 2 - RNA, 7856 nt, Homo sapiens
cagccgcccctcctgcggccgctgcgggggccgccgcctgacttcggacaccggccccgcacccgccaggaggggagggaaggggaggcggggagagcgacggcggggggcgggcggtggaccccgcctcccccggcacagcctgctgaggggaagagggggtctccgctcttcctcagtgcactctctgactgaagcccggcgcgtggggtgcagcgggagtgcgaggggactggacaggtgggaagatgggaatgaggaccgggcggcgggaatgttctcacttctccggattccaccgggatgcaggactctagctgcccagccgcacctgcgaagagactacacttcccgaggtgctcagcggcagcgagggcctccacgcatgcgcaccgcggcgcgctgggcggggctggatgggctgtggtgggagggttgcagcgccgcgagaaaggcgagccgggccgggggcggggaaaggggtggggcaggaacgggggcggggacggcgctggaggggcgggtcgggtaggtctcccggagctgatgtgtactgtgtgcgccggggaggcgccggcttgtactcggcagcgcgggaataaagtttgctgatttggtgtctagcctggatgcctgggttgcaggccctgcttgtggtggcgctccacagtcatccggctgaagaagacctgttggactggatcttctcgggttttctttcagatattgttttgtatttacccatgaagacattgttttttggactctgcaaataggacatttcaaagatgagtgaaaaaaaattggaaacaactgcacagcagcggaaatgtcctgaatggatgaatgtgcagaataaaagatgtgctgtagaagaaagaaaggcatgtgttcggaagagtgtttttgaagatgacctccccttcttagaattcactggatccattgtgtatagttacgatgctagtgattgctctttcctgtcagaagatattagcatgagtctatcagatggggatgtggtgggatttgacatggagtggccaccattatacaatagagggaaacttggcaaagttgcactaattcagttgtgtgtttctgagagcaaatgttacttgttccacgtttcttccatgtcagtttttccccagggattaaaaatgttgcttgaaaataaagcagttaaaaaggcaggtgtaggaattgaaggagatcagtggaaacttctacgtgactttgatatcaaattgaagaattttgtggagttgacagatgttgccaataaaaagctgaaatgcacagagacctggagccttaacagtctggttaaacacctcttaggtaaacagctcctgaaagacaagtctatccgctgtagcaattggagtaaatttcctctcactgaggaccagaaactgtatgcagccactgatgcttatgctggttttattatttaccgaaatttagagattttggatgatactgtgcaaaggtttgctataaataaagaggaagaaatcctacttagcgacatgaacaaacagttgacttcaatctctgaggaagtgatggatctggctaagcatcttcctcatgctttcagtaaattggaaaacccacggagggtttctatcttactaaaggatatttcagaaaatctatattcactgaggaggatgataattgggtctactaacattgagactgaactgaggcccagcaataatttaaacttattatcctttgaagattcaactactgggggagtacaacagaaacaaattagagaacatgaagttttaattcacgttgaagatgaaacatgggacccaacacttgatcatttagctaaacatgatggagaagatgtacttggaaataaagtggaacgaaaagaagatggatttgaagatggagtagaagacaacaaattgaaagagaatatggaaagagcttgtttgatgtcgttagatattacagaacatgaactccaaattttggaacagcagtctcaggaagaatatcttagtgatattgcttataaatctactgagcatttatctcccaatgataatgaaaacgatacgtcctatgtaattgagagtgatgaagatttagaaatggagatgcttaagcatttatctcccaatgataatgaaaacgatacgtcctatgtaattgagagtgatgaagatttagaaatggagatgcttaagtctttagaaaacctcaatagtggcacggtagaaccaactcattctaaatgcttaaaaatggaaagaaatctgggtcttcctactaaagaagaagaagaagatgatgaaaatgaagctaatgaaggggaagaagatgatgataaggactttttgtggccagcacccaatgaagagcaagttacttgcctcaagatgtactttggccattccagttttaaaccagttcagtggaaagtgattcattcagtattagaagaaagaagagataatgttgctgtcatggcaactggatatggaaagagtttgtgcttccagtatccacctgtttatgtaggcaagattggccttgttatctctccccttatttctctgatggaagaccaagtgctacagcttaaaatgtccaacatcccagcttgcttccttggatcagcacagtcagaaaatgttctaacagatattaaattaggtaaataccggattgtatacgtaactccagaatactgttcaggtaacatgggcctgctccagcaacttgaggctgatattggtatcacgctcattgctgtggatgaggctcactgtatttctgagtgggggcatgattttagggattcattcaggaagttgggctccctaaagacagcactgccaatggttccaatcgttgcacttactgctactgcaagttcttcaatccgggaagacattgtacgttgcttaaatctgagaaatcctcagatcacctgtactggttttgatcgaccaaacctgtatttagaagttaggcgaaaaacagggaatatccttcaggatctgcagccatttcttgtcaaaacaagttcccactgggaatttgaaggtccaacaatcatctactgtccttctagaaaaatgacacaacaagttacaggtgaacttaggaaactgaatctatcctgtggaacataccatgcgggcatgagttttagcacaaggaaagacattcatcataggtttgtaagagatgaaattcagtgtgtcatagctaccatagcttttggaatgggcattaataaagctgacattcgccaagtcattcattacggtgctcctaaggacatggaatcatattatcaggagattggtagagctggtcgtgatggacttcaaagttcttgtcacgtcctctgggctcctgcagacattaacttaaataggcaccttcttactgagatacgtaatgagaagtttcgattatacaaattaaagatgatggcaaagatggaaaaatatcttcattctagcagatgtaggagacaaatcatcttgtctcattttgaggacaaacaagtacaaaaagcctccttgggaattatgggaactgaaaaatgctgtgataattgcaggtccagattggatcattgctattccatggatgactcagaggatacatcctgggactttggtccacaagcatttaagcttttgtctgctgtggacatcttaggcgaaaaatttggaattgggcttccaattttatttctccgaggatctaattctcagcgtcttgccgatcaatatcgcaggcacagtttatttggcactggcaaggatcaaacagagagttggtggaaggctttttcccgtcagctgatcactgagggattcttggtagaagtttctcggtataacaaatttatgaagatttgcgcccttacgaaaaagggtagaaattggcttcataaagctaatacagaatctcagagcctcatccttcaagctaatgaagaattgtgtccaaagaagttgcttctgcctagttcgaaaactgtatcttcgggcaccaaagagcattgttataatcaagtaccagttgaattaagtacagagaagaagtctaacttggagaagttatattcttataaaccatgtgataagatttcttctgggagtaacatttctaaaaaaagtatcatggtacagtcaccagaaaaagcttacagttcctcacagcctgttatttcggcacaagagcaggagactcagattgtgttatatggcaaattggtagaagctaggcagaaacatgccaataaaatggatgttcccccagctattctggcaacaaacaagatactggtggatatggccaaaatgagaccaactacggttgaaaacgtaaaaaggattgatggtgtttctgaaggcaaagctgccatgttggcccctctgttggaagtcatcaaacatttctgccaaacaaatagtgttcagacagacctcttttcaagtacaaaacctcaagaagaacagaagacgagtctggtagcaaaaaataaaatatgcacactttcacagtctatggccatcacatactctttattccaagaaaagaagatgcctttgaagagcatagctgagagcaggattctgcctctcatgacaattggcatgcacttatcccaagcggtgaaagctggctgcccccttgatttggagcgagcaggcctgactccagaggttcagaagattattgctgatgttatccgaaaccctcccgtcaactcagatatgagtaaaattagcctaatcagaatgttagttcctgaaaacattgacacgtaccttatccacatggcaattgagatccttaaacatggtcctgacagcggacttcaaccttcatgtgatgtcaacaaaaggagatgttttcccggttctgaagagatctgttcaagttctaagagaagcaaggaagaagtaggcatcaatactgagacttcatctgcagagagaaagagacgattacctgtgtggtttgccaaaggaagtgataccagcaagaaattaatggacaaaacgaaaaggggaggtctttttagttaagctggcaattaccagaacaattatgtttcttgctgtattataagaggatagctatattttatttctgaagagtaaggagtagtattttggcttaaaaatcattctaattacaaagttcactgtttattgaagaactggcatcttaaatcagccttccgcaattcatgtagtttctgggtcttctgggagcctacgtgagtacatcacctaacagaatattaaattagacttcctgtaagattgctttaagaaactgttactgtcctgttttctaatctctttattaaaacagtgtatttggaaaatgttatgtgctctgatttgatatagataacagattagtagttacatggtaattatgtgatataaaatattcatatattatcaaaattctgttttgtaaatgtaagaaagcatagttattttacaaattgtttttactgtcttttgaagaagttcttaaatacgttgttaaatggtattagttgaccagggcagtgaaaatgaaaccgcattttgggtgccattaaatagggaaaaaacatgtaaaaaatgtaaaatggagaccaattgcactaggcaagtgtatattttgtattttatatacaatttctattatttttcaagtaataaaacaatgtttttcatactgaatattatatatatattttttagctttcatttacttaattattttaagtacctttatttttccaggatgtcagaatttgattctaatctctcttatgtagcacatgtgacttaatttaaaacctatactgtgacacagagttgggtaaacgatgattatttaactttaagcagttcaccatccatttcaaagcctttgattggcttttttgtaaataaaaataacttgttaagaaacaaatatatctgtcatagaagaactagaaaatccagggaagtgagaaaaatgaaaataaaaatcattcatagttttactagtagctaatcacagtcaacctcttttgtgtatcccaccagacttttttatattcatttgtttttagttaaaatataaaagtctcgtatattcccatttttctgcattgcattaccagaaggtagtggcgcctattaaatatgtgatatgttgttgtccagccatggcttctgcatttgcatgcttttgtgtgtgcatctgcaataccctgtgaatatcctgtgtgatggagtggcaagtacgcacagacacgtctgctgcatgcctaggtacgaggctgtctccaggagaagcacttgtttgattatttgagttgccaattgaatttgctgctttttttcatggcttgccattttcactgaaaagaatgactaatgaaaaacgatgattggttattagatttggatgtttggcagacattttctcaaaattgaactaagttggcctcttcacggaaaacaactggtatttgttgtgccaatgataaaattggagatttctagcaaaatgtataattttggaaaagttgtgttcctccactggaagcttgacagctttccttaacataaagacttctctttctcttcgctttcactactactactactaattcttcttctgattcttcttcttctccttcttccttcttccttccttcctcctcctcctccttcttcttcctcttcctcttcttctttctctctttccttccttcccttcccttccccttccttccttccttccttccttcctccctccctccctccctccctccctccctcctttctttttctttctctttctttctttctttctctctctctctctctttctttctttttctttctctttttctttctttcaagcagtcctcccgcctcagtcccccaaaatagtgggattacaggtgtgagccaccatgcacagccttacataaagccttttctaatgagatggatagtaattaacaaatgtgagtttttgatattatataaagattttttctgtgtttcgaagatccgtataactcagtgaatcagtatgttctggatgactaatatgtgatgttaagaaatcatgactgaggccgggcgcggtggctcacgcctgtaatcccagcactttgggaggccgaggcgggcggatcacgagatcaggagatcgagaccaccctggccaacatggtgaaaccccgtctctactaaaaatacaaaaattagctgggtgtgttggtgcgtgcctataatcccagctactcgggaggctgaggcaggagaatcgcttgaactcaggaggcggagattgcagtgagctgagactgcgccactgcaccccagcctggcgacagagcaagactccgtctcaaaaataaaaaaagaaatcatgactgggtaaaagatctgttcagagtacaagatggaccaatggatttgatatatttgaatataacagagtatgaaaaagtttattgatatagtttcagattacacactgcaactaatctttaagaaactattacttgtccactttttggtaaaatttcagagaacaatgtccaccattatctgaacaggctattaaaatactcttctcttttccaactacgtgcctgtgcaaagtcagatttttttcatatacttcagccaaaacagcatatcaaaatggattgaatgcagaagtagatctgagaatacagccacttttgttaagccagacaatgagatttgcaaaatgtaaacaatgctgctgttctcagtttttaaaaatatgttttttaaaagtatttatgttaatgtgtactt

SEQ ID NO: 3 - protein, 2285 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQNYKRPMDGTYGPPAKRHEGEMYSVPYSTGQGQPQQQQLPPAQPQPASQQQAAQPSPQQDVYNQYGNAYPATATAATERRPAGGPQNQFPFQFGRDRVSAPPGTNAQQNMPPQMMGGPIQASAEVAQQGTMWQGRNDMTYNYANRQSTGSAPQGPAYHGVNRTDEMLHTDQRANHEGSWPSHGTRQPPYGPSAPVPPMTRPPPSNYQPPPSMQNHIPQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 4 - RNA, 8595 nt, Homo sapiens
ctcctttctccggcagcagaaagcggagagtcacagcggggccaggccctggggagcggagcctccaccgcccccctcattcccaggcaagggcttggggggaatgagccgggagagccgggtcccgagcctacagagccgggagcagctgagccgccggcgcctcggccgccgccgccgcctcctcctcctccgccgccgccagcccggagcctgagccggcggggcgggggggagaggagcgagcgcagcgcagcagcggagccccgcgaggcccgcccgggcgggtggggagggcagcccgggggactgggccccggggcggggtgggagggggggagaagacgaagacagggccgggtctctccgcggacgagacagcggggatcatggccgcgcaggtcgcccccgccgccgccagcagcctgggcaacccgccgccgccgccgccctcggagctgaagaaagccgagcagcagcagcgggaggaggcggggggcgaggcggcggcggcggcagcggccgagcgcggggaaatgaaggcagccgccgggcaggaaagcgagggccccgccgtggggccgccgcagccgctgggaaaggagctgcaggacggggccgagagcaatgggggtggcggcggcggcggagccggcagcggcggcgggcccggcgcggagccggacctgaagaactcgaacgggaacgcgggccctaggcccgccctgaacaataacctcacggagccgcccggcggcggcggtggcggcagcagcgatggggtgggggcgcctcctcactcagccgcggccgccttgccgcccccagcctacggcttcgggcaaccctacggccggagcccgtctgccgtcgccgccgccgcggccgccgtcttccaccaacaacatggcggacaacaaagccctggcctggcagcgctgcagagcggcggcggcgggggcctggagccctacgcggggccccagcagaactctcacgaccacggcttccccaaccaccagtacaactcctactaccccaaccgcagcgcctaccccccgcccgccccggcctacgcgctgagctccccgagaggtggcactccgggctccggcgcggcggcggctgccggctccaagccgcctccctcctccagcgcctccgcctcctcgtcgtcttcgtccttcgctcagcagcgcttcggggccatggggggaggcggcccctccgcggccggcgggggaactccccagcccaccgccacccccaccctcaaccaactgctcacgtcgcccagctcggcccggggctaccagggctaccccgggggcgactacagtggcgggccccaggacgggggcgccggcaagggcccggcggacatggcctcgcagtgttggggggctgcggcggcggcagctgcggcggcggccgcctcgggaggggcccaacaaaggagccaccacgcgcccatgagccccgggagcagcggcggcggggggcagccgctcgcccggacccctcagccatccagtccaatggatcagatgggcaagatgagacctcagccatatggcgggactaacccatactcgcagcaacagggacctccgtcaggaccgcagcaaggacatgggtacccagggcagccatacgggtcccagaccccgcagcggtacccgatgaccatgcagggccgggcgcagagtgccatgggcggcctctcttatacacagcagattcctccttatggacaacaaggccccagcgggtatggtcaacagggccagactccatattacaaccagcaaagtcctcaccctcagcagcagcagccaccctactcccagcaaccaccgtcccagacccctcatgcccaaccttcgtatcagcagcagccacagtctcaaccaccacagctccagtcctctcagcctccatactcccagcagccatcccagcctccacatcagcagtccccggctccatacccctcccagcagtcgacgacacagcagcacccccagagccagcccccctactcacagccacaggctcagtctccttaccagcagcagcaacctcagcagccagcaccctcgacgctctcccagcaggctgcgtatcctcagccccagtctcagcagtcccagcaaactgcctattcccagcagcgcttccctccaccgcaggagctatctcaagattcatttgggtctcaggcatcctcagccccctcaatgacctccagtaagggagggcaagaagatatgaacctgagccttcagtcaagaccctccagcttgcctgatctatctggttcaatagatgacctccccatggggacagaaggagctctgagtcctggagtgagcacatcagggatttccagcagccaaggagagcagagtaatccagctcagtctcctttctctcctcatacctcccctcacctgcctggcatccgaggcccttccccgtcccctgttggctctcccgccagtgttgctcagtctcgctcaggaccactctcgcctgctgcagtgccaggcaaccagatgccacctcggccacccagtggccagtcggacagcatcatgcatccttccatgaaccaatcaagcattgcccaagatcgaggttatatgcagaggaacccccagatgccccagtacagttccccccagcccggctcagccttatctccgcgtcagccttccggaggacagatacacacaggcatgggctcctaccagcagaactccatggggagctatggtccccaggggggtcagtatggcccacaaggtggctaccccaggcagccaaactataatgccttgcccaatgccaactaccccagtgcaggcatggctggaggcataaaccccatgggtgccggaggtcaaatgcatggacagcctggcatcccaccttatggcacactccctccagggaggatgagtcacgcctccatgggcaaccggccttatggccctaacatggccaatatgccacctcaggttgggtcagggatgtgtcccccaccagggggcatgaaccggaaaacccaagaaactgctgtcgccatgcatgttgctgccaactctatccaaaacaggccgccaggctaccccaatatgaatcaagggggcatgatgggaactggacctccttatggacaagggattaatagtatggctggcatgatcaaccctcagggacccccatattccatgggtggaaccatggccaacaattctgcagggatggcagccagcccagagatgatgggccttggggatgtaaagttaactccagccaccaaaatgaacaacaaggcagatgggacacccaagacagaatccaaatccaagaaatccagttcttctactacaaccaatgagaagatcaccaagttgtatgagctgggtggtgagcctgagaggaagatgtgggtggaccgttatctggccttcactgaggagaaggccatgggcatgacaaatctgcctgctgtgggtaggaaacctctggacctctatcgcctctatgtgtctgtgaaggagattggtggattgactcaggtcaacaagaacaaaaaatggcgggaacttgcaaccaacctcaatgtgggcacatcaagcagtgctgccagctccttgaaaaagcagtatatccagtgtctctatgcctttgaatgcaagattgaacggggagaagaccctcccccagacatctttgcagctgctgattccaagaagtcccagcccaagatccagcctccctctcctgcgggatcaggatctatgcaggggccccagactccccagtcaaccagcagttccatggcagaaggaggagacttaaagccaccaactccagcatccacaccacacagtcagatccccccattgccaggcatgagcaggagcaattcagttgggatccaggatgcctttaatgatggaagtgactccacattccagaagcggaattccatgactccaaaccctgggtatcagcccagtatgaatacctctgacatgatggggcgcatgtcctatgagccaaataaggatccttatggcagcatgaggaaagctccagggagtgatcccttcatgtcctcagggcagggccccaacggcgggatgggtgacccctacagtcgtgctgccggccctgggctaggaaatgtggcgatgggaccacgacagcactatccctatggaggtccttatgacagagtgaggacggagcctggaatagggcctgagggaaacatgagcactggggccccacagccgaatctcatgccttccaacccagactcggggatgtattctcctagccgctaccccccgcagcagcagcagcagcagcagcaacgacatgattcctatggcaatcagttctccacccaaggcaccccttctggcagccccttccccagccagcagactacaatgtatcaacagcaacagcagaattacaagcggccaatggatggcacatatggccctcctgccaagcggcacgaaggggagatgtacagcgtgccatacagcactgggcaggggcagcctcagcagcagcagttgcccccagcccagccccagcctgccagccagcaacaagctgcccagccttcccctcagcaagatgtatacaaccagtatggcaatgcctatcctgccactgccacagctgctactgagcgccgaccagcaggcggcccccagaaccaatttccattccagtttggccgagaccgtgtctctgcaccccctggcaccaatgcccagcaaaacatgccaccacaaatgatgggcggccccatacaggcatcagctgaggttgctcagcaaggcaccatgtggcaggggcgtaatgacatgacctataattatgccaacaggcagagcacgggctctgccccccagggccccgcctatcatggcgtgaaccgaacagatgaaatgctgcacacagatcagagggccaaccacgaaggctcgtggccttcccatggcacacgccagcccccatatggtccctctgcccctgtgccccccatgacaaggccccctccatctaactaccagcccccaccaagcatgcagaatcacattcctcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccatgattcggcgggatatcaccttcccacctggctctgttgaagccacacagcctgtgttgaagcagaggaggcggctcacaatgaaagacattggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagagctccttgtagaatatttccgacgatgcctgattgagatctttggcattttaaaggagtatgaggtgggtgacccaggacagagaacgctactggatcctgggaggttcagcaaggtgtctagtccagctcccatggagggtggggaagaagaagaagaacttctaggtcctaaactagaagaggaagaagaagaggaagtagttgaaaatgatgaggagatagccttttcaggcaaggacaagccagcttcagagaatagtgaggagaagctgatcagtaagtttgacaagcttccagtaaagatcgtacagaagaatgatccatttgtggtggactgctcagataagcttgggcgtgtgcaggagtttgacagtggcctgctgcactggcggattggtgggggggacaccactgagcatatccagacccacttcgagagcaagacagagctgctgccttcccggcctcacgcaccctgcccaccagcccctcggaagcatgtgacaacagcagagggtacaccagggacaacagaccaggaggggcccccacctgatggacctccagaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaaggggtgagctgcaacaaagtggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaatacaaaaaaaaattctgaaggacaaaaaaggtgactgctgaactgtgtgtggtttattgttgtacattcacaatcttgcaggagccaagaagttcgcagttgtgaacagaccctgttcactggagaggcctgtgcagtagagtgtagaccctttcatgtactgtactgtacacctgatactgtaaacatactgtaataataatgtctcacatggaaacagaaaacgctgggtcagcagcaagctgtagtttttaaaaatgtttttagttaaacgttgaggagaaaaaaaaaaaaggcttttcccccaaagtatcatgtgtgaacctacaacaccctgacctctttctctcctccttgattgtatgaataaccctgagatcacctcttagaactggttttaacctttagctgcagcggctacgctgccacgtgtgtatatatatgacgttgtacattgcacatacccttggatccccacagtttggtcctcctcccagctacccctttatagtatgacgagttaacaagttggtgacctgcacaaagcgagacacagctatttaatctcttgccagatatcgcccctcttggtgcgatgctgtacaggtctctgtaaaaagtccttgctgtctcagcagccaatcaacttatagtttatttttttctgggtttttgttttgttttgttttctttctaatcgaggtgtgaaaaagttctaggttcagttgaagttctgatgaagaaacacaattgagattttttcagtgataaaatctgcatatttgtatttcaacaatgtagctaaaacttgatgtaaattcctcctttttttccttttttggcttaatgaatatcatttattcagtatgaaatctttatactatatgttccacgtgttaagaataaatgtacattaaatcttggtaa

SEQ ID NO: 5 - RNA, 20 nt, Artificial Sequence
  Synthetic Construct
gtaaattggaaaacccacgg

SEQ ID NO: 6 - RNA, 20 nt, Artificial Sequence
  Synthetic Construct
atcctgtggaacataccatg

SEQ ID NO: 7 - RNA, 20 nt, Artificial Sequence
  Synthetic Construct
gtagcagtaagtgcaacgat

SEQ ID NO: 8 - RNA, 20 nt, Artificial Sequence
  Synthetic Construct
cattacgtatctcagtaaga

SEQ ID NO: 9 - RNA, 20 nt, Artificial Sequence
  Synthetic Construct
ggacatggaatcatattatc

SEQ ID NO: 10 - RNA, 20 nt, Artificial Sequence
  Synthetic Construct
gagcaccgtaatgaatgact

SEQ ID NO: 11 - RNA, 22 nt, Artificial Sequence
  Synthetic Construct
uucuucaaaucacagugaacuc

SEQ ID NO: 12 - RNA, 22 nt, Artificial Sequence
  Synthetic Construct
uaauauucacauuggugauguu

SEQ ID NO: 13 - RNA, 22 nt, Artificial Sequence
  Synthetic Construct
uuacauuuagaacacgguuuuu

SEQ ID NO: 14 - RNA, 22 nt, Artificial Sequence
  Synthetic Construct
gcacauaagaaccucguaacuc

SEQ ID NO: 15 - RNA, 22 nt, Artificial Sequence
  Synthetic Construct
uucuuacaccaagucuacugua

SEQ ID NO: 16 - RNA, 22 nt, Artificial Sequence
  Synthetic Construct
uauacacuccgcuagugcagug

SEQ ID NO: 17 - RNA, 22 nt, Artificial Sequence
  Synthetic Construct
uucuguuaccaguugcacuccc

SEQ ID NO: 18 - RNA, 22 nt, Artificial Sequence
  Synthetic Construct
uauuuaauccgccgcuaggugc

SEQ ID NO: 19 - RNA, 22 nt, Artificial Sequence
  Synthetic Construct
cuauaucaguacagugcccugu

SEQ ID NO: 20 - RNA, 22 nt, Artificial Sequence
  Synthetic Construct
aaaauauacgguugcccugugc

SEQ ID NO: 21 - RNA, 19 nt, Artificial Sequence
  Synthetic Construct
ccatgaagacattgttttt

SEQ ID NO: 22 - RNA, 19 nt, Artificial Sequence
  Synthetic Construct
gccttaacagtctggttaa

SEQ ID NO: 23 - RNA, 19 nt, Artificial Sequence
  Synthetic Construct
gcactaggcaagtgtatat

SEQ ID NO: 24 - RNA, 19 nt, Artificial Sequence
  Synthetic Construct
ggatgaatgtgcagaataa

SEQ ID NO: 25 - RNA, 19 nt, Artificial Sequence
  Synthetic Construct
gacagatgttgccaataaa

SEQ ID NO: 26 - RNA, 19 nt, Artificial Sequence
  Synthetic Construct
cagatattgttttgtattt

SEQ ID NO: 27 - RNA, 19 nt, Artificial Sequence
  Synthetic Construct
catggagtggccaccatta

SEQ ID NO: 28 - RNA, 19 nt, Artificial Sequence
  Synthetic Construct
ctttcagtaaattggaaaa

SEQ ID NO: 29 - RNA, 19 nt, Artificial Sequence
  Synthetic Construct
ggaaaaaacatgtaaaaaa

SEQ ID NO: 30 - RNA, 19 nt, Artificial Sequence
  Synthetic Construct
gtaataaaacaatgttttt

SEQ ID NO: 31 - RNA, 19 nt, Artificial Sequence
  Synthetic Construct
gcaaaggtttgctataaat

SEQ ID NO: 32 - RNA, 19 nt, Artificial Sequence
  Synthetic Construct
gggtttctatcttactaaa

SEQ ID NO: 33 - RNA, 19 nt, Artificial Sequence
  Synthetic Construct
gaataaagtttgctgattt

SEQ ID NO: 34 - RNA, 19 nt, Artificial Sequence
  Synthetic Construct
gaaaaaacatgtaaaaaat

SEQ ID NO: 35 - RNA, 19 nt, Artificial Sequence
  Synthetic Construct
ctattatttttcaagtaat

SEQ ID NO: 36 - RNA, 19 nt, Artificial Sequence
  Synthetic Construct
cccatgaagacattgtttt

SEQ ID NO: 37 - RNA, 19 nt, Artificial Sequence
  Synthetic Construct
gcttatgctggttttatta

SEQ ID NO: 38 - RNA, 19 nt, Artificial Sequence
  Synthetic Construct
caaagatgagtgaaaaaaa

SEQ ID NO: 39 - RNA, 19 nt, Artificial Sequence
  Synthetic Construct
gtaagaaagcatagttatt

SEQ ID NO: 40 - RNA, 19 nt, Artificial Sequence
  Synthetic Construct
cttctacgtgactttgata

SEQ ID NO: 41 - RNA, 49 nt, Artificial Sequence
  Synthetic Construct
gaagatattagcatgagtctatcaagagtagactcatgctaatatcttc

SEQ ID NO: 42 - RNA, 49 nt, Artificial Sequence
  Synthetic Construct
gtggaaacttctacgtgactttcaagagaagtcacgtagaagtttccac

SEQ ID NO: 43 - RNA, 49 nt, Artificial Sequence
  Synthetic Construct
gagacctggagccttaacagttcaagagactgttaaggctccaggtctc

SEQ ID NO: 44 - RNA, 49 nt, Artificial Sequence
  Synthetic Construct
gcaactggatatggaaagagttcaagagactctttccatatccagttgc

SEQ ID NO: 45 - RNA, 49 nt, Artificial Sequence
  Synthetic Construct
gttccaatcgttgcacttacttcaagagagtaagtgcaacgattggaac

SEQ ID NO: 46 - RNA, 49 nt, Artificial Sequence
  Synthetic Construct
gacattgtacgttgcttaaattcaagagatttaagcaacgtacaatgtc

SEQ ID NO: 47 - RNA, 49 nt, Artificial Sequence
  Synthetic Construct
gcaccaaagagcattgttatatcaagagtataacaatgctctttggtgc

SEQ ID NO: 48 - RNA, 49 nt, Artificial Sequence
  Synthetic Construct
gtaggcatcaatactgagacttcaagagagtctcagtattgatgcctac

SEQ ID NO: 49 - RNA, 49 nt, Artificial Sequence
  Synthetic Construct
gtttcgaagatccgtataacttcaagagagttatacggatcttcgaaac

SEQ ID NO: 50 - RNA, 49 nt, Artificial Sequence
  Synthetic Construct
gtatgttctggatgactaatatcaagagtattagtcatccagaacatac

SEQ ID NO: 51 - RNA, 20 nt, Artificial Sequence
  Synthetic Construct
aagatcgagtgccgcatcac

SEQ ID NO: 52 - RNA, 20 nt, Artificial Sequence
  Synthetic Construct
tggaaacaactgcacagcag

SEQ ID NO: 53 - RNA, 20 nt, Artificial Sequence
  Synthetic Construct
agcagcggaaatgtcctgaa

SEQ ID NO: 54 - RNA, 20 nt, Artificial Sequence
  Synthetic Construct
ttctgcacattcatccattc

SEQ ID NO: 55 - RNA, 20 nt, Artificial Sequence
  Synthetic Construct
atgtgctgtagaagaaagaa

SEQ ID NO: 56 - RNA, 20 nt, Artificial Sequence
  Synthetic Construct
ctaaactcaaggcatgtgtt

SEQ ID NO: 57 - RNA, 20 nt, Artificial Sequence
  Synthetic Construct
cagtgaattctaagaagggg

SEQ ID NO: 58 - RNA, 20 nt, Artificial Sequence
  Synthetic Construct
atccagtgaattctaagaag

SEQ ID NO: 59 - RNA, 20 nt, Artificial Sequence
  Synthetic Construct
gatccagtgaattctaagaa

SEQ ID NO: 60 - RNA, 20 nt, Artificial Sequence
  Synthetic Construct
ggatccagtgaattctaaga

SEQ ID NO: 61 - RNA, 20 nt, Artificial Sequence
  Synthetic Construct
ctccccttcttagaattcac

SEQ ID NO: 62 - RNA, 20 nt, Artificial Sequence
  Synthetic Construct
agcatcgtaactatacacaa

SEQ ID NO: 63 - RNA, 20 nt, Artificial Sequence
  Synthetic Construct
cttacctaatatcttctgac

SEQ ID NO: 64 - RNA, 20 nt, Artificial Sequence
  Synthetic Construct
ctttcctgtcagaagatatt

SEQ ID NO: 65 - RNA, 20 nt, Artificial Sequence
  Synthetic Construct
tttagcatgagtctatcaga

SEQ ID NO: 66 - RNA, 20 nt, Artificial Sequence
  Synthetic Construct
ttagcatgagtctatcagat

SEQ ID NO: 67 - RNA, 20 nt, Artificial Sequence
  Synthetic Construct
tagcatgagtctatcagatg

SEQ ID NO: 68 - RNA, 20 nt, Artificial Sequence
  Synthetic Construct
gagtctatcagatggggatg

SEQ ID NO: 69 - RNA, 20 nt, Artificial Sequence
  Synthetic Construct
tctatcagatggggatgtgg

SEQ ID NO: 70 - RNA, 20 nt, Artificial Sequence
  Synthetic Construct
ctatcagatggggatgtggt

SEQ ID NO: 71 - RNA, 20 nt, Artificial Sequence
  Synthetic Construct
ggatgtggtgggatttgaca

SEQ ID NO: 72 - RNA, 20 nt, Artificial Sequence
  Synthetic Construct
tggtgggatttgacatggag

SEQ ID NO: 73 - RNA, 20 nt, Artificial Sequence
  Synthetic Construct
ttccctctattgtataatgg

SEQ ID NO: 74 - RNA, 20 nt, Artificial Sequence
  Synthetic Construct
tggccaccattatacaatag

SEQ ID NO: 75 - RNA, 20 nt, Artificial Sequence
  Synthetic Construct
ggccaccattatacaataga

SEQ ID NO: 76 - RNA, 20 nt, Artificial Sequence
  Synthetic Construct
aacctgacatggaagaaacg

SEQ ID NO: 77 - RNA, 20 nt, Artificial Sequence
  Synthetic Construct
ttccacgtttcttccatgtc

SEQ ID NO: 78 - RNA, 20 nt, Artificial Sequence
  Synthetic Construct
gtagagataccaacctgaca

SEQ ID NO: 79 - RNA, 20 nt, Artificial Sequence
  Synthetic Construct
acgtttcttccatgtcaggt

SEQ ID NO: 80 - RNA, 20 nt, Artificial Sequence
  Synthetic Construct
aataaagcagttaaaaaggc

SEQ ID NO: 81 - RNA, 20 nt, Artificial Sequence
  Synthetic Construct
gcagttaaaaaggcaggtgt

SEQ ID NO: 82 - RNA, 20 nt, Artificial Sequence
  Synthetic Construct
aaggcaggtgtaggaattga

SEQ ID NO: 83 - RNA, 20 nt, Artificial Sequence
  Synthetic Construct
taggaattgaaggagatcag

SEQ ID NO: 84 - RNA, 20 nt, Artificial Sequence
  Synthetic Construct
gacagatgttgccaataaaa

SEQ ID NO: 85 - RNA, 20 nt, Artificial Sequence
  Synthetic Construct
aaccagactgttaaggctcc

SEQ ID NO: 86 - RNA, 20 nt, Artificial Sequence
  Synthetic Construct
ggtgtttaaccagactgtta

SEQ ID NO: 87 - RNA, 20 nt, Artificial Sequence
  Synthetic Construct
agtctggttaaacacctctt

SEQ ID NO: 88 - RNA, 20 nt, Artificial Sequence
  Synthetic Construct
tcaggagctgtttacctaag

SEQ ID NO: 89 - RNA, 20 nt, Artificial Sequence
  Synthetic Construct
agcggatagacttgtctttc

SEQ ID NO: 90 - RNA, 20 nt, Artificial Sequence
  Synthetic Construct
atttactccaattgctacag

SEQ ID NO: 91 - RNA, 20 nt, Artificial Sequence
  Synthetic Construct
agtctatccgctgtagcaat

SEQ ID NO: 92 - RNA, 20 nt, Artificial Sequence
  Synthetic Construct
agtttctggtcctcagtgag

SEQ ID NO: 93 - RNA, 20 nt, Artificial Sequence
  Synthetic Construct
gagtaaatttcctctcactg

SEQ ID NO: 94 - RNA, 20 nt, Artificial Sequence
  Synthetic Construct
cagtggctgcatacagtttc

SEQ ID NO: 95 - RNA, 20 nt, Artificial Sequence
  Synthetic Construct
agcacgtacataagcatcag

SEQ ID NO: 96 - RNA, 20 nt, Artificial Sequence
  Synthetic Construct
catccaaaatctctaaattt

SEQ ID NO: 97 - RNA, 20 nt, Artificial Sequence
  Synthetic Construct
ttaccgaaatttagagattt

SEQ ID NO: 98 - RNA, 20 nt, Artificial Sequence
  Synthetic Construct
caaaggtttgctataaataa

SEQ ID NO: 99 - RNA, 20 nt, Artificial Sequence
  Synthetic Construct
taagtaggatttcttcctct

SEQ ID NO: 100 - RNA, 20 nt, Artificial Sequence
  Synthetic Construct
gtttgttcatgtcgctaagt

SEQ ID NO: 101 - RNA, 20 nt, Artificial Sequence
  Synthetic Construct
acagttgacttcaatctctg

SEQ ID NO: 102 - RNA, 20 nt, Artificial Sequence
  Synthetic Construct
ttcaatctctgaggaagtga

SEQ ID NO: 103 - RNA, 20 nt, Artificial Sequence
  Synthetic Construct
ctctgaggaagtgatggatc

SEQ ID NO: 104 - RNA, 20 nt, Artificial Sequence
  Synthetic Construct
tccaatttactgaaagcatg

SEQ ID NO: 105 - RNA, 20 nt, Artificial Sequence
  Synthetic Construct
tcctcatgctttcagtaaat

SEQ ID NO: 106 - RNA, 20 nt, Artificial Sequence
  Synthetic Construct
tcagtaaattggaaaaccca

SEQ ID NO: 107 - RNA, 20 nt, Artificial Sequence
  Synthetic Construct
gtaaattggaaaacccacgg

SEQ ID NO: 108 - RNA, 20 nt, Artificial Sequence
  Synthetic Construct
aggtaatatttaacctccgt

SEQ ID NO: 109 - RNA, 20 nt, Artificial Sequence
  Synthetic Construct
aaggtaatatttaacctccg

SEQ ID NO: 110 - RNA, 20 nt, Artificial Sequence
  Synthetic Construct
tagggtttctatcttactaa

SEQ ID NO: 111 - RNA, 20 nt, Artificial Sequence
  Synthetic Construct
cagaaaatctatattcactg

SEQ ID NO: 112 - RNA, 20 nt, Artificial Sequence
  Synthetic Construct
aaaatctatattcactgagg

SEQ ID NO: 113 - RNA, 20 nt, Artificial Sequence
  Synthetic Construct
tcactgaggaggatgataat

SEQ ID NO: 114 - RNA, 20 nt, Artificial Sequence
  Synthetic Construct
cactgaggaggatgataatt

SEQ ID NO: 115 - RNA, 20 nt, Artificial Sequence
  Synthetic Construct
ctaacattgagactgaactg

SEQ ID NO: 116 - RNA, 20 nt, Artificial Sequence
  Synthetic Construct
aataagtttaaattattgct

SEQ ID NO: 117 - RNA, 20 nt, Artificial Sequence
  Synthetic Construct
taataagtttaaattattgc

SEQ ID NO: 118 - RNA, 20 nt, Artificial Sequence
  Synthetic Construct
cccagtagttgaatcttcaa

SEQ ID NO: 119 - RNA, 20 nt, Artificial Sequence
  Synthetic Construct
tcctttgaagattcaactac

SEQ ID NO: 120 - RNA, 20 nt, Artificial Sequence
  Synthetic Construct
cctttgaagattcaactact

SEQ ID NO: 121 - RNA, 20 nt, Artificial Sequence
  Synthetic Construct
ctttgaagattcaactactg

SEQ ID NO: 122 - RNA, 20 nt, Artificial Sequence
  Synthetic Construct
tttgaagattcaactactgg

SEQ ID NO: 123 - RNA, 20 nt, Artificial Sequence
  Synthetic Construct
ttcacgttgaagatgaaaca

SEQ ID NO: 124 - RNA, 20 nt, Artificial Sequence
  Synthetic Construct
tcacgttgaagatgaaacat

SEQ ID NO: 125 - RNA, 20 nt, Artificial Sequence
  Synthetic Construct
tagctaaatgatcaagtgtt

SEQ ID NO: 126 - RNA, 20 nt, Artificial Sequence
  Synthetic Construct
ttagctaaatgatcaagtgt

SEQ ID NO: 127 - RNA, 20 nt, Artificial Sequence
  Synthetic Construct
gatcatttagctaaacatga

SEQ ID NO: 128 - RNA, 20 nt, Artificial Sequence
  Synthetic Construct
catgatggagaagatgtact

SEQ ID NO: 129 - RNA, 20 nt, Artificial Sequence
  Synthetic Construct
agatgtacttggaaataaag

SEQ ID NO: 130 - RNA, 20 nt, Artificial Sequence
  Synthetic Construct
caacaaattgaaagagaata

SEQ ID NO: 131 - RNA, 20 nt, Artificial Sequence
  Synthetic Construct
gagactgctgttccaaaatt

SEQ ID NO: 132 - RNA, 20 nt, Artificial Sequence
  Synthetic Construct
tattgcttataaatctactg

SEQ ID NO: 133 - RNA, 20 nt, Artificial Sequence
  Synthetic Construct
ttcatcactctcaattacat

SEQ ID NO: 134 - RNA, 20 nt, Artificial Sequence
  Synthetic Construct
gagtgatgaagatttagaaa

SEQ ID NO: 135 - RNA, 20 nt, Artificial Sequence
  Synthetic Construct
tttagaaatggagatgctta

SEQ ID NO: 136 - RNA, 20 nt, Artificial Sequence
  Synthetic Construct
gttctaccgtgccactattg

SEQ ID NO: 137 - RNA, 20 nt, Artificial Sequence
  Synthetic Construct
tctttagaaaacctcaatag

SEQ ID NO: 138 - RNA, 20 nt, Artificial Sequence
  Synthetic Construct
agaaaacctcaatagtggca

SEQ ID NO: 139 - RNA, 20 nt, Artificial Sequence
  Synthetic Construct
tttaagcatttagaatgagt

SEQ ID NO: 140 - RNA, 20 nt, Artificial Sequence
  Synthetic Construct
tcattctaaatgcttaaaaa

SEQ ID NO: 141 - RNA, 20 nt, Artificial Sequence
  Synthetic Construct
cttaaaaatggaaagaaatc

SEQ ID NO: 142 - RNA, 20 nt, Artificial Sequence
  Synthetic Construct
ttaaaaatggaaagaaatct

SEQ ID NO: 143 - RNA, 20 nt, Artificial Sequence
  Synthetic Construct
gatgaaaatgaagctaatga

SEQ ID NO: 144 - RNA, 20 nt, Artificial Sequence
  Synthetic Construct
atgaaaatgaagctaatgaa

SEQ ID NO: 145 - RNA, 20 nt, Artificial Sequence
  Synthetic Construct
tgaaaatgaagctaatgaag

SEQ ID NO: 146 - RNA, 20 nt, Artificial Sequence
  Synthetic Construct
ggtgctggccacaaaaagtc

SEQ ID NO: 147 - RNA, 20 nt, Artificial Sequence
  Synthetic Construct
acttgctcttcattgggtgc

SEQ ID NO: 148 - RNA, 20 nt, Artificial Sequence
  Synthetic Construct
caagtaacttgctcttcatt

SEQ ID NO: 149 - RNA, 20 nt, Artificial Sequence
  Synthetic Construct
gcaagtaacttgctcttcat

SEQ ID NO: 150 - RNA, 20 nt, Artificial Sequence
  Synthetic Construct
aatggccaaagtacatcttg

SEQ ID NO: 151 - RNA, 20 nt, Artificial Sequence
  Synthetic Construct
acttgcctcaagatgtactt

SEQ ID NO: 152 - RNA, 20 nt, Artificial Sequence
  Synthetic Construct
ctcacggtttaaaactggaa

SEQ ID NO: 153 - RNA, 20 nt, Artificial Sequence
  Synthetic Construct
ttatactcacggtttaaaac

SEQ ID NO: 154 - RNA, 20 nt, Artificial Sequence
  Synthetic Construct
tttcaccttcaagagttcag

SEQ ID NO: 155 - RNA, 20 nt, Artificial Sequence
  Synthetic Construct
aagagataatgttgctgtca

SEQ ID NO: 156 - RNA, 20 nt, Artificial Sequence
  Synthetic Construct
aatgttgctgtcatggcaac

SEQ ID NO: 157 - RNA, 20 nt, Artificial Sequence
  Synthetic Construct
ctacataaacaggtggatac

SEQ ID NO: 158 - RNA, 20 nt, Artificial Sequence
  Synthetic Construct
ccaatcttgcctacataaac

SEQ ID NO: 159 - RNA, 20 nt, Artificial Sequence
  Synthetic Construct
cagtatccacctgtttatgt

SEQ ID NO: 160 - RNA, 20 nt, Artificial Sequence
  Synthetic Construct
aaataaggggagagataaca

SEQ ID NO: 161 - RNA, 20 nt, Artificial Sequence
  Synthetic Construct
gtcttccatcagagaaataa

SEQ ID NO: 162 - RNA, 20 nt, Artificial Sequence
  Synthetic Construct
ggtcttccatcagagaaata

SEQ ID NO: 163 - RNA, 20 nt, Artificial Sequence
  Synthetic Construct
ctctccccttatttctctga

SEQ ID NO: 164 - RNA, 20 nt, Artificial Sequence
  Synthetic Construct
cttacttaagctgtagcact

SEQ ID NO: 165 - RNA, 20 nt, Artificial Sequence
  Synthetic Construct
aaggaagcaagctgggatgt

SEQ ID NO: 166 - RNA, 20 nt, Artificial Sequence
  Synthetic Construct
ctgatccaaggaagcaagct

SEQ ID NO: 167 - RNA, 20 nt, Artificial Sequence
  Synthetic Construct
gctgatccaaggaagcaagc

SEQ ID NO: 168 - RNA, 20 nt, Artificial Sequence
  Synthetic Construct
aacatcccagcttgcttcct

SEQ ID NO: 169 - RNA, 20 nt, Artificial Sequence
  Synthetic Construct
tttctgactgtgctgatcca

SEQ ID NO: 170 - RNA, 20 nt, Artificial Sequence
  Synthetic Construct
ctggagttacgtatacaatc

SEQ ID NO: 171 - RNA, 20 nt, Artificial Sequence
  Synthetic Construct
gtaactccagaatactgttc

SEQ ID NO: 172 - RNA, 20 nt, Artificial Sequence
  Synthetic Construct
agaatactgttcaggtaaca

SEQ ID NO: 173 - RNA, 20 nt, Artificial Sequence
  Synthetic Construct
cagcctcaagttgctggagc

SEQ ID NO: 174 - RNA, 20 nt, Artificial Sequence
  Synthetic Construct
caatatcagcctcaagttgc

SEQ ID NO: 175 - RNA, 20 nt, Artificial Sequence
  Synthetic Construct
gggcctgctccagcaacttg

SEQ ID NO: 176 - RNA, 20 nt, Artificial Sequence
  Synthetic Construct
cagcaacttgaggctgatat

SEQ ID NO: 177 - RNA, 20 nt, Artificial Sequence
  Synthetic Construct
aggtatcacgctcattgctg

SEQ ID NO: 178 - RNA, 20 nt, Artificial Sequence
  Synthetic Construct
cacgctcattgctgtggatg

SEQ ID NO: 179 - RNA, 20 nt, Artificial Sequence
  Synthetic Construct
aggctcactgtatttctgag

SEQ ID NO: 180 - RNA, 20 nt, Artificial Sequence
  Synthetic Construct
ggctcactgtatttctgagt

SEQ ID NO: 181 - RNA, 20 nt, Artificial Sequence
  Synthetic Construct
gctcactgtatttctgagtg

SEQ ID NO: 182 - RNA, 20 nt, Artificial Sequence
  Synthetic Construct
ctcactgtatttctgagtgg

SEQ ID NO: 183 - RNA, 20 nt, Artificial Sequence
  Synthetic Construct
tagggattcattcaggaagt

SEQ ID NO: 184 - RNA, 20 nt, Artificial Sequence
  Synthetic Construct
agggattcattcaggaagtt

SEQ ID NO: 185 - RNA, 20 nt, Artificial Sequence
  Synthetic Construct
cattggcagtgctgtcttta

SEQ ID NO: 186 - RNA, 20 nt, Artificial Sequence
  Synthetic Construct
ccattggcagtgctgtcttt

SEQ ID NO: 187 - RNA, 20 nt, Artificial Sequence
  Synthetic Construct
cctaaagacagcactgccaa

SEQ ID NO: 188 - RNA, 20 nt, Artificial Sequence
  Synthetic Construct
gacttggcaaagcttaccat

SEQ ID NO: 189 - RNA, 20 nt, Artificial Sequence
  Synthetic Construct
gtagcagtaagtgcaacgat

SEQ ID NO: 190 - RNA, 20 nt, Artificial Sequence
  Synthetic Construct
ctactgcaagttcttcaatc

SEQ ID NO: 191 - RNA, 20 nt, Artificial Sequence
  Synthetic Construct
tactgcaagttcttcaatcc

SEQ ID NO: 192 - RNA, 20 nt, Artificial Sequence
  Synthetic Construct
aaaccagtacaggtgatctg

SEQ ID NO: 193 - RNA, 20 nt, Artificial Sequence
  Synthetic Construct
aatcctcagatcacctgtac

SEQ ID NO: 194 - RNA, 20 nt, Artificial Sequence
  Synthetic Construct
tggtcgatcaaaaccagtac

SEQ ID NO: 195 - RNA, 20 nt, Artificial Sequence
  Synthetic Construct
ctaacttctaaatacaggtt

SEQ ID NO: 196 - RNA, 20 nt, Artificial Sequence
  Synthetic Construct
ttcgcctaacttctaaatac

SEQ ID NO: 197 - RNA, 20 nt, Artificial Sequence
  Synthetic Construct
caaacctgtatttagaagtt

SEQ ID NO: 198 - RNA, 20 nt, Artificial Sequence
  Synthetic Construct
ttagaagttaggcgaaaaac

SEQ ID NO: 199 - RNA, 20 nt, Artificial Sequence
  Synthetic Construct
tagaagttaggcgaaaaaca

SEQ ID NO: 200 - RNA, 20 nt, Artificial Sequence
  Synthetic Construct
aaaaacagggaatatccttc

SEQ ID NO: 201 - RNA, 20 nt, Artificial Sequence
  Synthetic Construct
gaaatggctgcagatcctga

SEQ ID NO: 202 - RNA, 20 nt, Artificial Sequence
  Synthetic Construct
agccatttcttgtcaaaaca

SEQ ID NO: 203 - RNA, 20 nt, Artificial Sequence
  Synthetic Construct
tggaccttcaaattcccagt

SEQ ID NO: 204 - RNA, 20 nt, Artificial Sequence
  Synthetic Construct
ttggaccttcaaattcccag

SEQ ID NO: 205 - RNA, 20 nt, Artificial Sequence
  Synthetic Construct
agttcccactgggaatttga

SEQ ID NO: 206 - RNA, 20 nt, Artificial Sequence
  Synthetic Construct
gaaggacagtagatgattgt

SEQ ID NO: 207 - RNA, 20 nt, Artificial Sequence
  Synthetic Construct
aaaatgacacaacaagttac

SEQ ID NO: 208 - RNA, 20 nt, Artificial Sequence
  Synthetic Construct
aacaagttacaggtgaactt

SEQ ID NO: 209 - RNA, 20 nt, Artificial Sequence
  Synthetic Construct
aggaaactgaatctatcctg

SEQ ID NO: 210 - RNA, 20 nt, Artificial Sequence
  Synthetic Construct
gcccgcatggtatgttccac

SEQ ID NO: 211 - RNA, 20 nt, Artificial Sequence
  Synthetic Construct
atcctgtggaacataccatg

SEQ ID NO: 212 - RNA, 20 nt, Artificial Sequence
  Synthetic Construct
tcctgtggaacataccatgc

SEQ ID NO: 213 - RNA, 20 nt, Artificial Sequence
  Synthetic Construct
tgctaaaactcatgcccgca

SEQ ID NO: 214 - RNA, 20 nt, Artificial Sequence
  Synthetic Construct
caaggaaagacattcatcat

SEQ ID NO: 215 - RNA, 20 nt, Artificial Sequence
  Synthetic Construct
gtttgtaagagatgaaattc

SEQ ID NO: 216 - RNA, 20 nt, Artificial Sequence
  Synthetic Construct
aatgcccattccaaaagcta

SEQ ID NO: 217 - RNA, 20 nt, Artificial Sequence
  Synthetic Construct
gtcatagctaccatagcttt

SEQ ID NO: 218 - RNA, 20 nt, Artificial Sequence
  Synthetic Construct
gagcaccgtaatgaatgact

SEQ ID NO: 219 - RNA, 20 nt, Artificial Sequence
  Synthetic Construct
attcgccaagtcattcatta

SEQ ID NO: 220 - RNA, 20 nt, Artificial Sequence
  Synthetic Construct
cattcattacggtgctccta

SEQ ID NO: 221 - RNA, 20 nt, Artificial Sequence
  Synthetic Construct
taatatgattccatgtcctt

SEQ ID NO: 222 - RNA, 20 nt, Artificial Sequence
  Synthetic Construct
ttacggtgctcctaaggaca

SEQ ID NO: 223 - RNA, 20 nt, Artificial Sequence
  Synthetic Construct
ggacatggaatcatattatc

SEQ ID NO: 224 - RNA, 20 nt, Artificial Sequence
  Synthetic Construct
gaatcatattatcaggagat

SEQ ID NO: 225 - RNA, 20 nt, Artificial Sequence
  Synthetic Construct
tatcaggagattggtagagc

SEQ ID NO: 226 - RNA, 20 nt, Artificial Sequence
  Synthetic Construct
attggtagagctggtcgtga

SEQ ID NO: 227 - RNA, 20 nt, Artificial Sequence
  Synthetic Construct
aaagttcttgtcacgtcctc

SEQ ID NO: 228 - RNA, 20 nt, Artificial Sequence
  Synthetic Construct
aagttcttgtcacgtcctct

SEQ ID NO: 229 - RNA, 20 nt, Artificial Sequence
  Synthetic Construct
taatgtctgcaggagcccag

SEQ ID NO: 230 - RNA, 20 nt, Artificial Sequence
  Synthetic Construct
ctatttaagttaatgtctgc

SEQ ID NO: 231 - RNA, 20 nt, Artificial Sequence
  Synthetic Construct
ctgcagacattaacttaaat

SEQ ID NO: 232 - RNA, 20 nt, Artificial Sequence
  Synthetic Construct
cattacgtatctcagtaaga

SEQ ID NO: 233 - RNA, 20 nt, Artificial Sequence
  Synthetic Construct
attatacaaattaaagatga

SEQ ID NO: 234 - RNA, 20 nt, Artificial Sequence
  Synthetic Construct
attaaagatgatggcaaaga

SEQ ID NO: 235 - RNA, 20 nt, Artificial Sequence
  Synthetic Construct
atcttcattctagcagatgt

SEQ ID NO: 236 - RNA, 20 nt, Artificial Sequence
  Synthetic Construct
acaagtacaaaaagcctcct

SEQ ID NO: 237 - RNA, 20 nt, Artificial Sequence
  Synthetic Construct
caagtacaaaaagcctcctt

SEQ ID NO: 238 - RNA, 20 nt, Artificial Sequence
  Synthetic Construct
agttcccataattcccaagg

SEQ ID NO: 239 - RNA, 20 nt, Artificial Sequence
  Synthetic Construct
ttcagttcccataattccca

SEQ ID NO: 240 - RNA, 20 nt, Artificial Sequence
  Synthetic Construct
aaaagcctccttgggaatta

SEQ ID NO: 241 - RNA, 20 nt, Artificial Sequence
  Synthetic Construct
aaagcctccttgggaattat

SEQ ID NO: 242 - RNA, 20 nt, Artificial Sequence
  Synthetic Construct
aaaaatgctgtgataattgc

SEQ ID NO: 243 - RNA, 20 nt, Artificial Sequence
  Synthetic Construct
gctgtgataattgcaggtcc

SEQ ID NO: 244 - RNA, 20 nt, Artificial Sequence
  Synthetic Construct
attggatcattgctattcca

SEQ ID NO: 245 - RNA, 20 nt, Artificial Sequence
  Synthetic Construct
tgtatcctctgagtcatcca

SEQ ID NO: 246 - RNA, 20 nt, Artificial Sequence
  Synthetic Construct
ctattccatggatgactcag

SEQ ID NO: 247 - RNA, 20 nt, Artificial Sequence
  Synthetic Construct
atgactcagaggatacatcc

SEQ ID NO: 248 - RNA, 20 nt, Artificial Sequence
  Synthetic Construct
tgactcagaggatacatcct

SEQ ID NO: 249 - RNA, 20 nt, Artificial Sequence
  Synthetic Construct
tgcttgtggaccaaagtccc

SEQ ID NO: 250 - RNA, 20 nt, Artificial Sequence
  Synthetic Construct
gaggatacatcctgggactt

SEQ ID NO: 251 - RNA, 20 nt, Artificial Sequence
  Synthetic Construct
gacaaaagcttaaatgcttg

SEQ ID NO: 252 - RNA, 20 nt, Artificial Sequence
  Synthetic Construct
ttgtctgctgtggacatctt

SEQ ID NO: 253 - RNA, 20 nt, Artificial Sequence
  Synthetic Construct
gacatcttaggcgaaaaatt

SEQ ID NO: 254 - RNA, 20 nt, Artificial Sequence
  Synthetic Construct
ttaggcgaaaaatttggaat

SEQ ID NO: 255 - RNA, 20 nt, Artificial Sequence
  Synthetic Construct
taggcgaaaaatttggaatt

SEQ ID NO: 256 - RNA, 20 nt, Artificial Sequence
  Synthetic Construct
gatcctcggagaaataaaat

SEQ ID NO: 257 - RNA, 20 nt, Artificial Sequence
  Synthetic Construct
gatatatacttacagatcct

SEQ ID NO: 258 - RNA, 20 nt, Artificial Sequence
  Synthetic Construct
actgtgcctgcgatattgat

SEQ ID NO: 259 - RNA, 20 nt, Artificial Sequence
  Synthetic Construct
gtcttgccgatcaatatcgc

SEQ ID NO: 260 - RNA, 20 nt, Artificial Sequence
  Synthetic Construct
tatcgcaggcacagtttatt

SEQ ID NO: 261 - RNA, 20 nt, Artificial Sequence
  Synthetic Construct
aggcacagtttatttggcac

SEQ ID NO: 262 - RNA, 20 nt, Artificial Sequence
  Synthetic Construct
cagtttatttggcactggca

SEQ ID NO: 263 - RNA, 20 nt, Artificial Sequence
  Synthetic Construct
gcaaggatcaaacagagagt

SEQ ID NO: 264 - RNA, 20 nt, Artificial Sequence
  Synthetic Construct
aggatcaaacagagagttgg

SEQ ID NO: 265 - RNA, 20 nt, Artificial Sequence
  Synthetic Construct
tcaaacagagagttggtgga

SEQ ID NO: 266 - RNA, 20 nt, Artificial Sequence
  Synthetic Construct
tccctcagtgatcagctgac

SEQ ID NO: 267 - RNA, 20 nt, Artificial Sequence
  Synthetic Construct
atccctcagtgatcagctga

SEQ ID NO: 268 - RNA, 20 nt, Artificial Sequence
  Synthetic Construct
ttcccgtcagctgatcactg

SEQ ID NO: 269 - RNA, 20 nt, Artificial Sequence
  Synthetic Construct
tcccgtcagctgatcactga

SEQ ID NO: 270 - RNA, 20 nt, Artificial Sequence
  Synthetic Construct
gctgatcactgagggattct

SEQ ID NO: 271 - RNA, 20 nt, Artificial Sequence
  Synthetic Construct
gattcttggtagaagtttct

SEQ ID NO: 272 - RNA, 20 nt, Artificial Sequence
  Synthetic Construct
gatttgcgcccttacgaaaa

SEQ ID NO: 273 - RNA, 20 nt, Artificial Sequence
  Synthetic Construct
cttcattagcttgaaggatg

SEQ ID NO: 274 - RNA, 20 nt, Artificial Sequence
  Synthetic Construct
acaattcttcattagcttga

SEQ ID NO: 275 - RNA, 20 nt, Artificial Sequence
  Synthetic Construct
ctaggcagaagcaacttctt

SEQ ID NO: 276 - RNA, 20 nt, Artificial Sequence
  Synthetic Construct
caaagaagttgcttctgcct

SEQ ID NO: 277 - RNA, 20 nt, Artificial Sequence
  Synthetic Construct
tagttcgaaaactgtatctt

SEQ ID NO: 278 - RNA, 20 nt, Artificial Sequence
  Synthetic Construct
agttcgaaaactgtatcttc

SEQ ID NO: 279 - RNA, 20 nt, Artificial Sequence
  Synthetic Construct
ttgattataacaatgctctt

SEQ ID NO: 280 - RNA, 20 nt, Artificial Sequence
  Synthetic Construct
ttctctgtacttaattcaac

SEQ ID NO: 281 - RNA, 20 nt, Artificial Sequence
  Synthetic Construct
tgaattaagtacagagaaga

SEQ ID NO: 282 - RNA, 20 nt, Artificial Sequence
  Synthetic Construct
tgtttaaatgcagtctaact

SEQ ID NO: 283 - RNA, 20 nt, Artificial Sequence
  Synthetic Construct
ccagaagaaatcttatcaca

SEQ ID NO: 284 - RNA, 20 nt, Artificial Sequence
  Synthetic Construct
ccatgtgataagatttcttc

SEQ ID NO: 285 - RNA, 20 nt, Artificial Sequence
  Synthetic Construct
catgtgataagatttcttct

SEQ ID NO: 286 - RNA, 20 nt, Artificial Sequence
  Synthetic Construct
ggagtaacatttctaaaaaa

SEQ ID NO: 287 - RNA, 20 nt, Artificial Sequence
  Synthetic Construct
tgccgaaataacaggctgtg

SEQ ID NO: 288 - RNA, 20 nt, Artificial Sequence
  Synthetic Construct
tgctcttgtgccgaaataac

SEQ ID NO: 289 - RNA, 20 nt, Artificial Sequence
  Synthetic Construct
ttcctcacagcctgttattt

SEQ ID NO: 290 - RNA, 20 nt, Artificial Sequence
  Synthetic Construct
tgttatttcggcacaagagc

SEQ ID NO: 291 - RNA, 20 nt, Artificial Sequence
  Synthetic Construct
ggcacaagagcaggagactc

SEQ ID NO: 292 - RNA, 20 nt, Artificial Sequence
  Synthetic Construct
tttgttcagattgtgttata

SEQ ID NO: 293 - RNA, 20 nt, Artificial Sequence
  Synthetic Construct
gattgtgttatatggcaaat

SEQ ID NO: 294 - RNA, 20 nt, Artificial Sequence
  Synthetic Construct
atggcaaattggtagaagct

SEQ ID NO: 295 - RNA, 20 nt, Artificial Sequence
  Synthetic Construct
gcagaaacatgccaataaaa

SEQ ID NO: 296 - RNA, 20 nt, Artificial Sequence
  Synthetic Construct
tttgttgccagaatagctgg

SEQ ID NO: 297 - RNA, 20 nt, Artificial Sequence
  Synthetic Construct
gtttgttgccagaatagctg

SEQ ID NO: 298 - RNA, 20 nt, Artificial Sequence
  Synthetic Construct
tgtttgttgccagaatagct

SEQ ID NO: 299 - RNA, 20 nt, Artificial Sequence
  Synthetic Construct
ttgtttgttgccagaatagc

SEQ ID NO: 300 - RNA, 20 nt, Artificial Sequence
  Synthetic Construct
ggatgttcccccagctattc

SEQ ID NO: 301 - RNA, 20 nt, Artificial Sequence
  Synthetic Construct
tctggcaacaaacaagatac

SEQ ID NO: 302 - RNA, 20 nt, Artificial Sequence
  Synthetic Construct
ggcaacaaacaagatactgg

SEQ ID NO: 303 - RNA, 20 nt, Artificial Sequence
  Synthetic Construct
aaacaagatactggtggata

SEQ ID NO: 304 - RNA, 20 nt, Artificial Sequence
  Synthetic Construct
aaagatagtttacctcattt

SEQ ID NO: 305 - RNA, 20 nt, Artificial Sequence
  Synthetic Construct
tgttaaacacagaccaacta

SEQ ID NO: 306 - RNA, 20 nt, Artificial Sequence
  Synthetic Construct
ctacggttgaaaacgtaaaa

SEQ ID NO: 307 - RNA, 20 nt, Artificial Sequence
  Synthetic Construct
gaaaacgtaaaaaggattga

SEQ ID NO: 308 - RNA, 20 nt, Artificial Sequence
  Synthetic Construct
aggattgatggtgtttctga

SEQ ID NO: 309 - RNA, 20 nt, Artificial Sequence
  Synthetic Construct
tgaaggcaaagctgccatgt

SEQ ID NO: 310 - RNA, 20 nt, Artificial Sequence
  Synthetic Construct
ttccaacagaggggccaaca

SEQ ID NO: 311 - RNA, 20 nt, Artificial Sequence
  Synthetic Construct
tttgatgacttccaacagag

SEQ ID NO: 312 - RNA, 20 nt, Artificial Sequence
  Synthetic Construct
gtttgatgacttccaacaga

SEQ ID NO: 313 - RNA, 20 nt, Artificial Sequence
  Synthetic Construct
tgtttgatgacttccaacag

SEQ ID NO: 314 - RNA, 20 nt, Artificial Sequence
  Synthetic Construct
ttacctgaacactatttgtt

SEQ ID NO: 315 - RNA, 20 nt, Artificial Sequence
  Synthetic Construct
ctgccaaacaaatagtgttc

SEQ ID NO: 316 - RNA, 20 nt, Artificial Sequence
  Synthetic Construct
agaagaacagaagacgagtc

SEQ ID NO: 317 - RNA, 20 nt, Artificial Sequence
  Synthetic Construct
atgcacactttcacagtcta

SEQ ID NO: 318 - RNA, 20 nt, Artificial Sequence
  Synthetic Construct
ttggaataaagagtatgtga

SEQ ID NO: 319 - RNA, 20 nt, Artificial Sequence
  Synthetic Construct
ccaagaaaagaagatgcctt

SEQ ID NO: 320 - RNA, 20 nt, Artificial Sequence
  Synthetic Construct
atgaaagtcacacttaccaa

SEQ ID NO: 321 - RNA, 20 nt, Artificial Sequence
  Synthetic Construct
agaagagcatagctgagagc

SEQ ID NO: 322 - RNA, 20 nt, Artificial Sequence
  Synthetic Construct
tgcatgccaattgtcatgag

SEQ ID NO: 323 - RNA, 20 nt, Artificial Sequence
  Synthetic Construct
attctgcctctcatgacaat

SEQ ID NO: 324 - RNA, 20 nt, Artificial Sequence
  Synthetic Construct
tggcatgcacttatcccaag

SEQ ID NO: 325 - RNA, 20 nt, Artificial Sequence
  Synthetic Construct
gcagccagctttcaccgctt

SEQ ID NO: 326 - RNA, 20 nt, Artificial Sequence
  Synthetic Construct
ggcagccagctttcaccgct

SEQ ID NO: 327 - RNA, 20 nt, Artificial Sequence
  Synthetic Construct
ttatcccaagcggtgaaagc

SEQ ID NO: 328 - RNA, 20 nt, Artificial Sequence
  Synthetic Construct
ctgctcgctccaaatcaagg

SEQ ID NO: 329 - RNA, 20 nt, Artificial Sequence
  Synthetic Construct
cctgctcgctccaaatcaag

SEQ ID NO: 330 - RNA, 20 nt, Artificial Sequence
  Synthetic Construct
gcctgctcgctccaaatcaa

SEQ ID NO: 331 - RNA, 20 nt, Artificial Sequence
  Synthetic Construct
ggcctgctcgctccaaatca

SEQ ID NO: 332 - RNA, 20 nt, Artificial Sequence
  Synthetic Construct
ccccttgatttggagcgagc

SEQ ID NO: 333 - RNA, 20 nt, Artificial Sequence
  Synthetic Construct
gcgagcaggcctgactccag

SEQ ID NO: 334 - RNA, 20 nt, Artificial Sequence
  Synthetic Construct
gcaataatcttctgaacctc

SEQ ID NO: 335 - RNA, 20 nt, Artificial Sequence
  Synthetic Construct
ctgagttgacgggagggttt

SEQ ID NO: 336 - RNA, 20 nt, Artificial Sequence
  Synthetic Construct
tctcacctgagttgacggga

SEQ ID NO: 337 - RNA, 20 nt, Artificial Sequence
  Synthetic Construct
ctctcacctgagttgacggg

SEQ ID NO: 338 - RNA, 20 nt, Artificial Sequence
  Synthetic Construct
atgcctctcacctgagttga

SEQ ID NO: 339 - RNA, 20 nt, Artificial Sequence
  Synthetic Construct
cgaaaccctcccgtcaactc

SEQ ID NO: 340 - RNA, 20 nt, Artificial Sequence
  Synthetic Construct
caggaactaacattctgatt

SEQ ID NO: 341 - RNA, 20 nt, Artificial Sequence
  Synthetic Construct
tctcaattgccatgtggata

SEQ ID NO: 342 - RNA, 20 nt, Artificial Sequence
  Synthetic Construct
tgacacgtaccttatccaca

SEQ ID NO: 343 - RNA, 20 nt, Artificial Sequence
  Synthetic Construct
taaggatctcaattgccatg

SEQ ID NO: 344 - RNA, 20 nt, Artificial Sequence
  Synthetic Construct
cgctgtcaggaccatgttta

SEQ ID NO: 345 - RNA, 20 nt, Artificial Sequence
  Synthetic Construct
gcaattgagatccttaaaca

SEQ ID NO: 346 - RNA, 20 nt, Artificial Sequence
  Synthetic Construct
gaaggttgaagtccgctgtc

SEQ ID NO: 347 - RNA, 20 nt, Artificial Sequence
  Synthetic Construct
cttcatgtgatgtcaacaaa

SEQ ID NO: 348 - RNA, 20 nt, Artificial Sequence
  Synthetic Construct
gaacagatctcttcagaacc

SEQ ID NO: 349 - RNA, 20 nt, Artificial Sequence
  Synthetic Construct
tgaacagatctcttcagaac

SEQ ID NO: 350 - RNA, 20 nt, Artificial Sequence
  Synthetic Construct
ttcaagttctaagagaagca

SEQ ID NO: 351 - RNA, 20 nt, Artificial Sequence
  Synthetic Construct
agaagtaggcatcaatactg

SEQ ID NO: 352 - RNA, 20 nt, Artificial Sequence
  Synthetic Construct
gaaagagacgattacctgtg

SEQ ID NO: 353 - RNA, 20 nt, Artificial Sequence
  Synthetic Construct
cttcctttggcaaaccacac

SEQ ID NO: 354 - RNA, 20 nt, Artificial Sequence
  Synthetic Construct
ttacctgtgtggtttgccaa

SEQ ID NO: 355 - RNA, 20 nt, Artificial Sequence
  Synthetic Construct
cttgctggtatcacttcctt

SEQ ID NO: 356 - RNA, 20 nt, Artificial Sequence
  Synthetic Construct
tttgtccattaatttcttgc

SEQ ID NO: 357 - RNA, 20 nt, Artificial Sequence
  Synthetic Construct
tgataccagcaagaaattaa

SEQ ID NO: 358 - RNA, 20 nt, Artificial Sequence
  Synthetic Construct
aattaatggacaaaacgaaa

SEQ ID NO: 359 - RNA, 20 nt, Artificial Sequence
  Synthetic Construct
attaatggacaaaacgaaaa

SEQ ID NO: 360 - RNA, 20 nt, Artificial Sequence
  Synthetic Construct
ttaatggacaaaacgaaaag

SEQ ID NO: 361 - RNA, 20 nt, Artificial Sequence
  Synthetic Construct
atggacaaaacgaaaagggg

SEQ ID NO: 362 - RNA, 20 nt, Artificial Sequence
  Synthetic Construct
gtagcgaacgtgtccggcgt

SEQ ID NO: 363 - RNA, 20 nt, Artificial Sequence
  Synthetic Construct
gaccggaacgatctcgcgta

SEQ ID NO: 364 - RNA, 20 nt, Artificial Sequence
  Synthetic Construct
ggcagtcgttcggttgatat

SEQ ID NO: 365 - RNA, 20 nt, Artificial Sequence
  Synthetic Construct
gcttgagcacatacgcgaat

SEQ ID NO: 366 - RNA, 20 nt, Artificial Sequence
  Synthetic Construct
gtggtagaataacgtattac

SEQ ID NO: 367 - RNA, 20 nt, Artificial Sequence
  Synthetic Construct
gtcatacatggataaggcta

SEQ ID NO: 368 - RNA, 20 nt, Artificial Sequence
  Synthetic Construct
gatacacgaagcatcactag

SEQ ID NO: 369 - RNA, 20 nt, Artificial Sequence
  Synthetic Construct
gaacgttggcactacttcac

SEQ ID NO: 370 - RNA, 20 nt, Artificial Sequence
  Synthetic Construct
gatccatgtaatgcgttcga

SEQ ID NO: 371 - RNA, 20 nt, Artificial Sequence
  Synthetic Construct
gtcgtgaagtgcattcgatc

SEQ ID NO: 372 - RNA, 20 nt, Artificial Sequence
  Synthetic Construct
gttcgactcgcgtgaccgta

SEQ ID NO: 373 - RNA, 20 nt, Artificial Sequence
  Synthetic Construct
gaatctaccgcagcggttcg

SEQ ID NO: 374 - RNA, 20 nt, Artificial Sequence
  Synthetic Construct
gaagtgacgtcgattcgata

SEQ ID NO: 375 - RNA, 20 nt, Artificial Sequence
  Synthetic Construct
gcggtgtatgacaaccgccg

SEQ ID NO: 376 - RNA, 20 nt, Artificial Sequence
  Synthetic Construct
gtaccgcgcctgaagttcgc

SEQ ID NO: 377 - RNA, 20 nt, Artificial Sequence
  Synthetic Construct
gcagctcgtgtgtcgtactc

SEQ ID NO: 378 - RNA, 20 nt, Artificial Sequence
  Synthetic Construct
gcgccttaagagtactcatc

SEQ ID NO: 379 - RNA, 20 nt, Artificial Sequence
  Synthetic Construct
gagtgtcgtcgttgctccta

SEQ ID NO: 380 - RNA, 20 nt, Artificial Sequence
  Synthetic Construct
gcagctcgacctcaagccgt

SEQ ID NO: 381 - RNA, 20 nt, Artificial Sequence
  Synthetic Construct
gtatcctgacctacgcgctg

SEQ ID NO: 382 - RNA, 20 nt, Artificial Sequence
  Synthetic Construct
gtgtatctcagcacgctaac

SEQ ID NO: 383 - RNA, 20 nt, Artificial Sequence
  Synthetic Construct
gtcgtcatacaacggcaacg

SEQ ID NO: 384 - RNA, 20 nt, Artificial Sequence
  Synthetic Construct
gtcgtgcgcttccggcggta

SEQ ID NO: 385 - RNA, 20 nt, Artificial Sequence
  Synthetic Construct
gcggtcctcagtaagcgcgt

SEQ ID NO: 386 - RNA, 20 nt, Artificial Sequence
  Synthetic Construct
gctctgctgcggaaggattc

SEQ ID NO: 387 - RNA, 20 nt, Artificial Sequence
  Synthetic Construct
gcatggaggagcgtcgcaga

SEQ ID NO: 388 - RNA, 20 nt, Artificial Sequence
  Synthetic Construct
gtagcgcgcgtaggagtggc

SEQ ID NO: 389 - RNA, 20 nt, Artificial Sequence
  Synthetic Construct
gatcacctgcattcgtacac

SEQ ID NO: 390 - RNA, 20 nt, Artificial Sequence
  Synthetic Construct
gcacacctagatatcgaatg

SEQ ID NO: 391 - RNA, 20 nt, Artificial Sequence
  Synthetic Construct
gttgatcaacgcgcttcgcg

SEQ ID NO: 392 - RNA, 20 nt, Artificial Sequence
  Synthetic Construct
gcgtctcactcactccatcg

SEQ ID NO: 393 - RNA, 20 nt, Artificial Sequence
  Synthetic Construct
gccgaccaacgtcagcggta

SEQ ID NO: 394 - RNA, 20 nt, Artificial Sequence
  Synthetic Construct
ggatacggtgcgtcaatcta

SEQ ID NO: 395 - RNA, 20 nt, Artificial Sequence
  Synthetic Construct
gaatccagtggcggcgacaa

SEQ ID NO: 396 - RNA, 20 nt, Artificial Sequence
  Synthetic Construct
gcactgtcagtgcaacgata

SEQ ID NO: 397 - RNA, 20 nt, Artificial Sequence
  Synthetic Construct
gcgatcctcaagtatgctca

SEQ ID NO: 398 - RNA, 20 nt, Artificial Sequence
  Synthetic Construct
gctaatatcgacacggccgc

SEQ ID NO: 399 - RNA, 20 nt, Artificial Sequence
  Synthetic Construct
ggagatgcatcgaagtcgat

SEQ ID NO: 400 - RNA, 20 nt, Artificial Sequence
  Synthetic Construct
ggatgcactccatctcgtct

SEQ ID NO: 401 - RNA, 20 nt, Artificial Sequence
  Synthetic Construct
gtgccgagtaataacgcgag

SEQ ID NO: 402 - RNA, 20 nt, Artificial Sequence
  Synthetic Construct
gagattccgatgtaacgtac

SEQ ID NO: 403 - RNA, 20 nt, Artificial Sequence
  Synthetic Construct
gtcgtcacgagcaggattgc

SEQ ID NO: 404 - RNA, 20 nt, Artificial Sequence
  Synthetic Construct
gcgttagtcacttagctcga

SEQ ID NO: 405 - RNA, 20 nt, Artificial Sequence
  Synthetic Construct
gttcacacggtgtcggatag

SEQ ID NO: 406 - RNA, 20 nt, Artificial Sequence
  Synthetic Construct
ggataggtgaccttagtacg

SEQ ID NO: 407 - RNA, 20 nt, Artificial Sequence
  Synthetic Construct
gtatgagtcaagctaatgcg

SEQ ID NO: 408 - RNA, 20 nt, Artificial Sequence
  Synthetic Construct
gcaactattggaatacgtga

SEQ ID NO: 409 - RNA, 20 nt, Artificial Sequence
  Synthetic Construct
gttaccttcgctcgtctata

SEQ ID NO: 410 - RNA, 20 nt, Artificial Sequence
  Synthetic Construct
gtaccgagcaccacaggccg

SEQ ID NO: 411 - RNA, 20 nt, Artificial Sequence
  Synthetic Construct
gtcagccatcggatagagat

SEQ ID NO: 412 - RNA, 20 nt, Artificial Sequence
  Synthetic Construct
gtacggcactcctagccgct

SEQ ID NO: 413 - RNA, 20 nt, Artificial Sequence
  Synthetic Construct
ggtcctgtcgtatgcttgca

SEQ ID NO: 414 - RNA, 20 nt, Artificial Sequence
  Synthetic Construct
gccgcaatatatgcggtaag

SEQ ID NO: 415 - RNA, 20 nt, Artificial Sequence
  Synthetic Construct
gcgcacgtataatcctgcgt

SEQ ID NO: 416 - RNA, 20 nt, Artificial Sequence
  Synthetic Construct
gtgcacaacacgatccacga

SEQ ID NO: 417 - RNA, 20 nt, Artificial Sequence
  Synthetic Construct
gcacaatgttgacgtaagtg

SEQ ID NO: 418 - RNA, 20 nt, Artificial Sequence
  Synthetic Construct
gtaagatgctgctcaccgtg

SEQ ID NO: 419 - RNA, 20 nt, Artificial Sequence
  Synthetic Construct
gtcggtgatccaacgtatcg

SEQ ID NO: 420 - RNA, 20 nt, Artificial Sequence
  Synthetic Construct
gagctagtaggacgcaagac

SEQ ID NO: 421 - RNA, 20 nt, Artificial Sequence
  Synthetic Construct
gtacgtggaagcttgtggcc

SEQ ID NO: 422 - RNA, 20 nt, Artificial Sequence
  Synthetic Construct
gagaactgccagttctcgat

SEQ ID NO: 423 - RNA, 20 nt, Artificial Sequence
  Synthetic Construct
gccattcggcgcggcacttc

SEQ ID NO: 424 - RNA, 20 nt, Artificial Sequence
  Synthetic Construct
gcacacgaccaatccgcttc

SEQ ID NO: 425 - RNA, 20 nt, Artificial Sequence
  Synthetic Construct
gaggtgatcgattaagtaca

SEQ ID NO: 426 - RNA, 20 nt, Artificial Sequence
  Synthetic Construct
gtcactcgcagacgcctaac

SEQ ID NO: 427 - RNA, 20 nt, Artificial Sequence
  Synthetic Construct
gcgctacggaatcatacgtt

SEQ ID NO: 428 - RNA, 20 nt, Artificial Sequence
  Synthetic Construct
ggtaggacctcacggcgcgc

SEQ ID NO: 429 - RNA, 20 nt, Artificial Sequence
  Synthetic Construct
gaactgcatcttgttgtagt

SEQ ID NO: 430 - RNA, 20 nt, Artificial Sequence
  Synthetic Construct
gatcctgatccggcggcgcg

SEQ ID NO: 431 - RNA, 20 nt, Artificial Sequence
  Synthetic Construct
ggtatgcgcgatcctgagtt

SEQ ID NO: 432 - RNA, 20 nt, Artificial Sequence
  Synthetic Construct
gcggagctagagagcggtca

SEQ ID NO: 433 - RNA, 20 nt, Artificial Sequence
  Synthetic Construct
gaatggcaattacggctgat

SEQ ID NO: 434 - RNA, 20 nt, Artificial Sequence
  Synthetic Construct
gtatggtgagtagtcgcttg

SEQ ID NO: 435 - RNA, 20 nt, Artificial Sequence
  Synthetic Construct
gtgtaattgcgtctagtcgg

SEQ ID NO: 436 - RNA, 20 nt, Artificial Sequence
  Synthetic Construct
ggtcctggcgaggagccttg

SEQ ID NO: 437 - RNA, 20 nt, Artificial Sequence
  Synthetic Construct
gaagataagtcgctgtctcg

SEQ ID NO: 438 - RNA, 20 nt, Artificial Sequence
  Synthetic Construct
gtcggcgttctgttgtgact

SEQ ID NO: 439 - RNA, 20 nt, Artificial Sequence
  Synthetic Construct
gaggcaagccgttaggtgta

SEQ ID NO: 440 - RNA, 20 nt, Artificial Sequence
  Synthetic Construct
gcggatccagatctcattcg

SEQ ID NO: 441 - RNA, 20 nt, Artificial Sequence
  Synthetic Construct
ggaacataggagcacgtagt

SEQ ID NO: 442 - RNA, 20 nt, Artificial Sequence
  Synthetic Construct
gtcatcattatggcgtaagg

SEQ ID NO: 443 - RNA, 20 nt, Artificial Sequence
  Synthetic Construct
gcgactagcgccatgagcgg

SEQ ID NO: 444 - RNA, 20 nt, Artificial Sequence
  Synthetic Construct
ggcgaagttcgacatgacac

SEQ ID NO: 445 - RNA, 20 nt, Artificial Sequence
  Synthetic Construct
gctgtcgtgtggaggctatg

SEQ ID NO: 446 - RNA, 20 nt, Artificial Sequence
  Synthetic Construct
gcggagagcattgacctcat

SEQ ID NO: 447 - RNA, 20 nt, Artificial Sequence
  Synthetic Construct
gactaatggaccaagtcagt

SEQ ID NO: 448 - RNA, 20 nt, Artificial Sequence
  Synthetic Construct
gcggattagaggtaatgcgg

SEQ ID NO: 449 - RNA, 20 nt, Artificial Sequence
  Synthetic Construct
gccgacggcaatcagtacgc

SEQ ID NO: 450 - RNA, 20 nt, Artificial Sequence
  Synthetic Construct
gtaacctctcgagcgataga

SEQ ID NO: 451 - RNA, 20 nt, Artificial Sequence
  Synthetic Construct
gacttgtatgtggcttacgg

SEQ ID NO: 452 - RNA, 20 nt, Artificial Sequence
  Synthetic Construct
gtcactgtggtcgaacatgt

SEQ ID NO: 453 - RNA, 20 nt, Artificial Sequence
  Synthetic Construct
gtactccaatccgcgatgac

SEQ ID NO: 454 - RNA, 20 nt, Artificial Sequence
  Synthetic Construct
gcgttggcacgatgttacgg

SEQ ID NO: 455 - RNA, 20 nt, Artificial Sequence
  Synthetic Construct
gaaccagccggctagtatga

SEQ ID NO: 456 - RNA, 20 nt, Artificial Sequence
  Synthetic Construct
gtatactagctaaccacacg

SEQ ID NO: 457 - RNA, 20 nt, Artificial Sequence
  Synthetic Construct
gaatcggaatagttgattcg

SEQ ID NO: 458 - RNA, 20 nt, Artificial Sequence
  Synthetic Construct
gagcacttgcatgaggcggt

SEQ ID NO: 459 - RNA, 20 nt, Artificial Sequence
  Synthetic Construct
gaacggcgatgaagccagcc

SEQ ID NO: 460 - RNA, 20 nt, Artificial Sequence
  Synthetic Construct
gcaaccgagatgagaggttc

SEQ ID NO: 461 - RNA, 20 nt, Artificial Sequence
  Synthetic Construct
gcaagatcaatatgcgtgat

SEQ ID NO: 462 - RNA, 20 nt, Artificial Sequence
  Synthetic Construct
acggaggctaagcgtcgcaa

SEQ ID NO: 463 - RNA, 20 nt, Artificial Sequence
  Synthetic Construct
cgcttccgcggcccgttcaa

SEQ ID NO: 464 - RNA, 20 nt, Artificial Sequence
  Synthetic Construct
atcgtttccgcttaacggcg

SEQ ID NO: 465 - RNA, 20 nt, Artificial Sequence
  Synthetic Construct
gtaggcgcgccgctctctac

SEQ ID NO: 466 - RNA, 20 nt, Artificial Sequence
  Synthetic Construct
ccatatcggggcgagacatg

SEQ ID NO: 467 - RNA, 20 nt, Artificial Sequence
  Synthetic Construct
tactaacgccgctcctacag

SEQ ID NO: 468 - RNA, 20 nt, Artificial Sequence
  Synthetic Construct
tgaggatcatgtcgagcgcc

SEQ ID NO: 469 - RNA, 20 nt, Artificial Sequence
  Synthetic Construct
gggcccgcataggatatcgc

SEQ ID NO: 470 - RNA, 20 nt, Artificial Sequence
  Synthetic Construct
tagacaaccgcggagaatgc

SEQ ID NO: 471 - RNA, 20 nt, Artificial Sequence
  Synthetic Construct
acgggcggctatcgctgact

SEQ ID NO: 472 - RNA, 20 nt, Artificial Sequence
  Synthetic Construct
cgcggaaattttaccgacga

SEQ ID NO: 473 - RNA, 20 nt, Artificial Sequence
  Synthetic Construct
cttacaatcgtcggtccaat

SEQ ID NO: 474 - RNA, 20 nt, Artificial Sequence
  Synthetic Construct
gcgtgcgtcccgggttaccc

SEQ ID NO: 475 - RNA, 20 nt, Artificial Sequence
  Synthetic Construct
cggagtaacaagcggacgga

SEQ ID NO: 476 - RNA, 20 nt, Artificial Sequence
  Synthetic Construct
cgagtgttatacgcaccgtt

SEQ ID NO: 477 - RNA, 20 nt, Artificial Sequence
  Synthetic Construct
cgactaaccggaaacttttt

SEQ ID NO: 478 - RNA, 20 nt, Artificial Sequence
  Synthetic Construct
caacgggttctcccggctac

SEQ ID NO: 479 - RNA, 20 nt, Artificial Sequence
  Synthetic Construct
caggagtcgccgatacgcgt

SEQ ID NO: 480 - RNA, 20 nt, Artificial Sequence
  Synthetic Construct
ttcacgtcgtctcgcgacca

SEQ ID NO: 481 - RNA, 20 nt, Artificial Sequence
  Synthetic Construct
gtgtcggattccgccgctta

SEQ ID NO: 482 - RNA, 20 nt, Artificial Sequence
  Synthetic Construct
cacgaactcacaccgcgcga

SEQ ID NO: 483 - RNA, 20 nt, Artificial Sequence
  Synthetic Construct
cgctagtacgctcctctata

SEQ ID NO: 484 - RNA, 20 nt, Artificial Sequence
  Synthetic Construct
tcgcgcttgggttatacgct

SEQ ID NO: 485 - RNA, 20 nt, Artificial Sequence
  Synthetic Construct
ctatctcgagtggtaatgcg

SEQ ID NO: 486 - RNA, 20 nt, Artificial Sequence
  Synthetic Construct
aatcgactcgaacttcgtgt

SEQ ID NO: 487 - RNA, 20 nt, Artificial Sequence
  Synthetic Construct
cccgatggactataccgaac

SEQ ID NO: 488 - RNA, 20 nt, Artificial Sequence
  Synthetic Construct
acgttcgagtacgaccagct

SEQ ID NO: 489 - RNA, 20 nt, Artificial Sequence
  Synthetic Construct
cgcgacgactcaacctagtc

SEQ ID NO: 490 - RNA, 20 nt, Artificial Sequence
  Synthetic Construct
ggtcaccgatcgagagctag

SEQ ID NO: 491 - RNA, 20 nt, Artificial Sequence
  Synthetic Construct
ctcaaccgaccgtatggtca

SEQ ID NO: 492 - RNA, 20 nt, Artificial Sequence
  Synthetic Construct
cgtattcgactctcaacgcg

SEQ ID NO: 493 - RNA, 20 nt, Artificial Sequence
  Synthetic Construct
ctagccgcccagatcgagcc

SEQ ID NO: 494 - RNA, 20 nt, Artificial Sequence
  Synthetic Construct
gaatcgaccgacactaatgt

SEQ ID NO: 495 - RNA, 20 nt, Artificial Sequence
  Synthetic Construct
acttcagttcggcgtagtca

SEQ ID NO: 496 - RNA, 20 nt, Artificial Sequence
  Synthetic Construct
gtgcgatgtcgcttcaacgt

SEQ ID NO: 497 - RNA, 20 nt, Artificial Sequence
  Synthetic Construct
cgcctaatttccggatcaat

SEQ ID NO: 498 - RNA, 20 nt, Artificial Sequence
  Synthetic Construct
cgtggccggaaccgtcatag

SEQ ID NO: 499 - RNA, 20 nt, Artificial Sequence
  Synthetic Construct
accctccgaatcgtaacgga

SEQ ID NO: 500 - RNA, 20 nt, Artificial Sequence
  Synthetic Construct
aaacggtacgacagcgtgtg

SEQ ID NO: 501 - RNA, 20 nt, Artificial Sequence
  Synthetic Construct
acatagtcgacggctcgatt

SEQ ID NO: 502 - RNA, 20 nt, Artificial Sequence
  Synthetic Construct
gatggcgcttcagtcgtcgg

SEQ ID NO: 503 - RNA, 20 nt, Artificial Sequence
  Synthetic Construct
ataatccggaaacgctcgac

SEQ ID NO: 504 - RNA, 20 nt, Artificial Sequence
  Synthetic Construct
cgccgggctgacaattaacg

SEQ ID NO: 505 - RNA, 20 nt, Artificial Sequence
  Synthetic Construct
cgtcgccatatgccggtggc

SEQ ID NO: 506 - RNA, 20 nt, Artificial Sequence
  Synthetic Construct
cgggcctataacaccatcga

SEQ ID NO: 507 - RNA, 20 nt, Artificial Sequence
  Synthetic Construct
cgccgttccgagatacttga

SEQ ID NO: 508 - RNA, 20 nt, Artificial Sequence
  Synthetic Construct
cgggacgtcgcgaaaatgta

SEQ ID NO: 509 - RNA, 20 nt, Artificial Sequence
  Synthetic Construct
tcggcatacgggacacacgc

SEQ ID NO: 510 - RNA, 20 nt, Artificial Sequence
  Synthetic Construct
agctccatcgccgcgataat

SEQ ID NO: 511 - RNA, 20 nt, Artificial Sequence
  Synthetic Construct
atcgtatcatcagctagcgc

SEQ ID NO: 512 - RNA, 20 nt, Artificial Sequence
  Synthetic Construct
tcgatcgaggttgcattcgg

SEQ ID NO: 513 - RNA, 20 nt, Artificial Sequence
  Synthetic Construct
ctcgacagttcgtcccgagc

SEQ ID NO: 514 - RNA, 20 nt, Artificial Sequence
  Synthetic Construct
cggtagtattaatcgctgac

SEQ ID NO: 515 - RNA, 20 nt, Artificial Sequence
  Synthetic Construct
tgaacgcgtgtttccttgca

SEQ ID NO: 516 - RNA, 20 nt, Artificial Sequence
  Synthetic Construct
cgacgctaggtaacgtagag

SEQ ID NO: 517 - RNA, 20 nt, Artificial Sequence
  Synthetic Construct
cattgttgagcgggcgcgct

SEQ ID NO: 518 - RNA, 20 nt, Artificial Sequence
  Synthetic Construct
ccgctattgaaaccgcccac

SEQ ID NO: 519 - RNA, 20 nt, Artificial Sequence
  Synthetic Construct
agacacgtcaccggtcaaaa

SEQ ID NO: 520 - RNA, 20 nt, Artificial Sequence
  Synthetic Construct
tttacgatctagcggcgtag

SEQ ID NO: 521 - RNA, 20 nt, Artificial Sequence
  Synthetic Construct
ttcgcacgattgcaccttgg

SEQ ID NO: 522 - RNA, 20 nt, Artificial Sequence
  Synthetic Construct
ggttagagactaggcgcgcg

SEQ ID NO: 523 - RNA, 20 nt, Artificial Sequence
  Synthetic Construct
cctccgtgctaacgcggacg

SEQ ID NO: 524 - RNA, 20 nt, Artificial Sequence
  Synthetic Construct
ttatcgcgtagtgctgacgt

SEQ ID NO: 525 - RNA, 20 nt, Artificial Sequence
  Synthetic Construct
tacgcttgcgtttagcgtcc

SEQ ID NO: 526 - RNA, 20 nt, Artificial Sequence
  Synthetic Construct
cgcggcccacgcgtcatcgc

SEQ ID NO: 527 - RNA, 20 nt, Artificial Sequence
  Synthetic Construct
agctcgccatgtcggttctc

SEQ ID NO: 528 - RNA, 20 nt, Artificial Sequence
  Synthetic Construct
aactagcccgagcagcttcg

SEQ ID NO: 529 - RNA, 20 nt, Artificial Sequence
  Synthetic Construct
cgcaaggtgtcggtaaccct

SEQ ID NO: 530 - RNA, 20 nt, Artificial Sequence
  Synthetic Construct
cttcgacgccatcgtgctca

SEQ ID NO: 531 - RNA, 20 nt, Artificial Sequence
  Synthetic Construct
tcctggataccgcgtggtta

SEQ ID NO: 532 - RNA, 20 nt, Artificial Sequence
  Synthetic Construct
atagccgccgctcattactt

SEQ ID NO: 533 - RNA, 20 nt, Artificial Sequence
  Synthetic Construct
gtcgtccgggattacaaaat

SEQ ID NO: 534 - RNA, 20 nt, Artificial Sequence
  Synthetic Construct
taatgctgcacacgccgaat

SEQ ID NO: 535 - RNA, 20 nt, Artificial Sequence
  Synthetic Construct
tatcgcttccgattagtccg

SEQ ID NO: 536 - RNA, 20 nt, Artificial Sequence
  Synthetic Construct
gtaccataccgcgtaccctt

SEQ ID NO: 537 - RNA, 20 nt, Artificial Sequence
  Synthetic Construct
taagatccgcgggtggcaac

SEQ ID NO: 538 - RNA, 20 nt, Artificial Sequence
  Synthetic Construct
gtagacgtcgtgagcttcac

SEQ ID NO: 539 - RNA, 20 nt, Artificial Sequence
  Synthetic Construct
tcgcggacatagggctctaa

SEQ ID NO: 540 - RNA, 20 nt, Artificial Sequence
  Synthetic Construct
agcgcagatagcgcgtatca

SEQ ID NO: 541 - RNA, 20 nt, Artificial Sequence
  Synthetic Construct
gttcgcttcgtaacgaggaa

SEQ ID NO: 542 - RNA, 20 nt, Artificial Sequence
  Synthetic Construct
gacccccgataacttttgac

SEQ ID NO: 543 - RNA, 20 nt, Artificial Sequence
  Synthetic Construct
acgtccatactgtcggctac

SEQ ID NO: 544 - RNA, 20 nt, Artificial Sequence
  Synthetic Construct
gtaccattgccggctcccta

SEQ ID NO: 545 - RNA, 20 nt, Artificial Sequence
  Synthetic Construct
tggttccgtaggtcggtata

SEQ ID NO: 546 - RNA, 20 nt, Artificial Sequence
  Synthetic Construct
tctggcttgacacgaccgtt

SEQ ID NO: 547 - RNA, 20 nt, Artificial Sequence
  Synthetic Construct
cgctaggtccggtaagtgcg

SEQ ID NO: 548 - RNA, 20 nt, Artificial Sequence
  Synthetic Construct
agcacgtaatgtccgtggat

SEQ ID NO: 549 - RNA, 20 nt, Artificial Sequence
  Synthetic Construct
aaggcgcgcgaatgtggcag

SEQ ID NO: 550 - RNA, 20 nt, Artificial Sequence
  Synthetic Construct
actgcggagcgcccaatatc

SEQ ID NO: 551 - RNA, 20 nt, Artificial Sequence
  Synthetic Construct
cgtcgagtgctcgaactcca

SEQ ID NO: 552 - RNA, 20 nt, Artificial Sequence
  Synthetic Construct
tcgcagcggcgtgggatcgg

SEQ ID NO: 553 - RNA, 20 nt, Artificial Sequence
  Synthetic Construct
atctgtcctaattcggatcg

SEQ ID NO: 554 - RNA, 20 nt, Artificial Sequence
  Synthetic Construct
tgcggcgtaatgcttgaaag

SEQ ID NO: 555 - RNA, 20 nt, Artificial Sequence
  Synthetic Construct
cgaacttaatcccgtggcaa

SEQ ID NO: 556 - RNA, 20 nt, Artificial Sequence
  Synthetic Construct
gccgtgttgctggatacgcc

SEQ ID NO: 557 - RNA, 20 nt, Artificial Sequence
  Synthetic Construct
taccctccggatacggactg

SEQ ID NO: 558 - RNA, 20 nt, Artificial Sequence
  Synthetic Construct
ccgttggactatggcgggtc

SEQ ID NO: 559 - RNA, 20 nt, Artificial Sequence
  Synthetic Construct
gtacggggcgatcatccaca

SEQ ID NO: 560 - RNA, 20 nt, Artificial Sequence
  Synthetic Construct
aagagtagtagacgcccggg

SEQ ID NO: 561 - RNA, 20 nt, Artificial Sequence
  Synthetic Construct
aagagcgaatcgatttcgtg